(19) United States
(12) Patent Application Publication
(10) Pub. No.: US 2019/0023775 A1
(43) Pub. Date: Jan. 24, 2019

(54) DOSAGE AND ADMINISTRATION OF ANTI-C5 ANTIBODIES FOR TREATMENT

(71) Applicant: Alexion Pharmaceuticals, Inc., New Haven, CT (US)

(72) Inventors: Eric BACHMAN, Swampscott, MA (US); David MITCHELL, Lafayette, CO (US); Leonardo SAHELIJO, Swampscott, MA (US)

(21) Appl. No.: 16/068,453

(22) PCT Filed: Jan. 11, 2017
(86) PCT No.: PCT/US2017/013021

§ 371 Date: Jul. 6, 2018

Related U.S. Application Data
(60) Provisional application No. 62/315,761, filed on Mar. 31, 2016.
(60) Provisional application No. 62/367,782, filed on Jul. 28, 2016.
(60) Provisional application No. 62/426,849, filed on Nov. 28, 2016.
(60) Provisional application No. 62/367,695, filed on Jul. 28, 2016.
(60) Provisional application No. 62/328,724, filed on Apr. 28, 2016.
(60) Provisional application No. 62/378,520, filed on Aug. 23, 2016.
(60) Provisional application No. 62/346,658, filed on Jun. 7, 2016.
(60) Provisional application No. 62/277,317, filed on Jan. 11, 2016.

(51) Int. Cl. C07K 16/18; C07K 16/28; C07K 16/46; A61P 7/00

(52) U.S. Cl. CPC C07K 16/18; C07K 16/283; A61K 2039/505; A61P 7/00; C07K 2317/92; C07K 16/468

(57) ABSTRACT

Provided are methods for clinical treatment of Paroxysmal Nocturnal Hemoglobinuria (PNH) and atypical hemolytic uremic syndrome (aHUS) using an anti-C5 antibody, or antigen binding fragment thereof.

### RELATED APPLICATIONS

This application claims priority to U.S. Provisional Application No. 62/277,317 (filed Jan. 11, 2016), U.S. Provisional Application No. 62/315,761 (filed Mar. 31, 2016), U.S. Provisional Application No. 62/328,724 (filed Apr. 28, 2016), U.S. Provisional Application No. 62/346,658 (filed Jun. 7, 2016), U.S. Provisional Application No. 62/367,695 (filed Jul. 28, 2016), U.S. Provisional Application No. 62/367,782 (filed Jul. 28, 2016), U.S. Provisional Application No. 62/378,520 (filed Aug. 23, 2016), and U.S. Provisional Application No. 62/426,849 (filed Nov. 28, 2016). The contents of any patents, patent applications, and references cited throughout this specification are hereby incorporated by reference in their entireties.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

While a properly functioning complement system provides a robust defense against infecting microbes, inappropriate regulation or activation of the complement pathways has been implicated in the pathogenesis of a variety of disorders, including paroxysmal nocturnal hemoglobinuria (PNH) and atypical hemolytic uremic syndrome (aHUS). PNH and aHUS, are both ultra-rare disorders driven by chronic uncontrolled complement activation. The resulting inflammation and cellular damage lead to the devastating clinical manifestations of these diseases.

PNH is a condition in which uncontrolled complement activity leads to systemic complications, principally through intravascular hemolysis and platelet activation (see Socié G, et al., French Society of Haematology. Lancet. 1996; 348(9027):573-577 and Brodsky, R., Blood. 2014; 124(18):2804-2811). Persistent intravascular hemolysis may be triggered by various stressors, such as infection or physical exertion, and this leads to smooth muscle contraction (free hemoglobin), chronic anemia, and an increased risk of severe thromboembolism. Thromboembolism is the most common cause of mortality in patients with PNH, and pulmonary hypertension and end-organ damage of vital organs, such as the liver, kidneys, brain, and intestines, are sequelae of such events (Hillmen, P., et al, Am. J. Hematol. 2010; 85(8):553-559). Due to these adverse pathologic processes, patients with PNH have a decreased quality of life (QoL), which may include debilitating fatigue, chronic pain, poor physical function, shortness of breath, abdominal pain, erectile dysfunction, a need for anticoagulation, blood transfusions and in some instances, need for dialysis (Weitz, I C., et al., Thromb Res. 2012; 130(3):361-368).

Hemolytic uremic syndrome (HUS) is characterized by thrombocytopenia, microangiopathic hemolytic anemia, and acute renal failure. HUS is classified as one of two types: diarrheal-associated (D+ HUS; also referred to as shiga toxin producing E. coli (STEC)-HUS or typical HUS) and non-diarrheal or atypical HUS (aHUS). D+ HUS is the most common form, accounting for greater than 90% of cases and is caused by a preceding illness with a shiga-like toxin-producing bacterium, e.g., E. coli O157:H7.

aHUS can be genetic, acquired, or idiopathic. Hereditable forms of aHUS can be associated with mutations in a number of human complement components including, e.g., complement factor H (CFH), membrane cofactor protein (MCP), complement factor I (CFI), C4b-binding protein (C4BP), complement factor B (CFB), and complement component 3 (C3). See, e.g., Caprioli et al. (2006) Blood 108:1267-1279. Certain mutations in the gene encoding CD55, though not yet implicated in aHUS, are associated with the severity of aHUS. See, e.g., Esparza-Gordillo et al. (2005) Hum Mol Genet 14:703-712.

aHUS is rare and has a mortality rate of up to 25%. Many patients with this disease will sustain permanent neurological or renal impairment, e.g., at least 50% of aHUS patients progress to end-stage renal failure (ESRF). See, e.g., Kavanagh et al. (2006) British Medical Bulletin 77 and 78:5-22. Until recently, treatment options for patients with aHUS were limited and often involved plasma infusion or plasma exchange. In some cases, aHUS patients undergo uni- or bilateral nephrectomy or renal transplantation (see Artz et al. (2003) Transplantation 76:821-826). However, recurrence of the disease in treated patients is common.

Patients with PNH or aHUS are at risk of substantial morbidity and mortality. Accordingly, it is an object of the present invention to provide improved methods for treating patients with PNH or aHUS.

### SUMMARY

Provided herein are compositions and methods for treating Paroxysmal Nocturnal Hemoglobinuria (PNH) or atypical hemolytic uremic syndrome (aHUS) in a human patient, comprising administering to the patient an anti-C5 antibody, or antigen binding fragment thereof, wherein the anti-C5 antibody, or antigen binding fragment thereof, is administered (or is for administration) according to a particular clinical dosage regimen (i.e., at a particular dose amount and according to a specific dosing schedule). In one embodiment, the patient has not previously been treated with a complement inhibitor (e.g., the patient is a complement inhibitor treatment-naïve patient).

An exemplary anti-C5 antibody is antibody BNJ441 (also known as ALXN1210) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:12, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively.

In another embodiment, the antibody comprises VH and VL regions having the amino acid sequences set forth in SEQ ID NO:12 and SEQ ID NO:8, respectively.

In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 95% or 99% variable region identity with SEQ ID NO:12 and SEQ ID NO:8).

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

Accordingly, in one aspect, methods of treating a human patient are provided, the methods comprising administering to the patient an effective amount of an anti-C5 antibody, or antigen binding fragment thereof. In one embodiment, the patient has PNH. In another embodiment, the patient has aHUS.

In one embodiment, the dose of the anti-C5 antibody, or antigen binding fragment thereof, is a flat-fixed dose that is fixed irrespective of the weight of the patient. For example, the anti-C5 antibody, or antigen binding fragment thereof, may be administered at a fixed dose of 400, 600, 900, 1000, 1400, 1600, 1800, 2000, 2400, 3000, or 5400 mg, without regard to the patient's weight. In certain embodiments, dosage regimens are adjusted to provide the optimum desired response (e.g., an effective response).

In one embodiment, the anti-C5 antibody, or antigen binding fragment thereof, is administered twice during the induction phase. For example, the anti-C5 antibody, or antigen binding fragment thereof, is administered on Day 1 and Day 15 of the administration cycle. In one embodiment, the anti-C5 antibody, or antigen binding fragment thereof, is administered at a dose of 600 mg on Day 1 of the administration cycle and at a dose of 600 mg on Day 15 of the administration cycle. In another embodiment, the anti-C5 antibody, or antigen binding fragment thereof, is administered at a dose of 600 mg on Day 1 of the administration cycle and at a dose of 900 mg on Day 15 of the administration cycle. In one embodiment, the administration cycle comprises a period of 21 weeks.

In another embodiment, the anti-C5 antibody, or antigen binding fragment thereof, is administered three times during the induction phase. For example, the anti-C5 antibody, or antigen binding fragment thereof, is administered on Day 1, Day 8, and Day 15 of the administration cycle. In one embodiment, the anti-C5 antibody, or antigen binding fragment thereof, is administered at a dose of 400 mg on Day 1 of the administration cycle, a dose of 400 mg on Day 8 of the administration cycle, and a dose of 600 mg on Day 15 of the administration cycle.

In another embodiment, the anti-C5 antibody, or antigen binding fragment thereof, is administered five times during the maintenance phase. For example, the anti-C5 antibody, or antigen binding fragment thereof, is administered on Day 29 of the administration cycle and then every 28 days thereafter, i.e., on Days 57, 85, 113, and 141, for a total of five doses. In one embodiment, the anti-C5 antibody, or antigen binding fragment thereof, is administered at a dose of 900 mg on Days 29, 57, 85, 113, and 141. In another embodiment, the anti-C5 antibody, or antigen binding fragment thereof, is administered at a dose of 1800 mg on Days 29, 57, 85, 113, and 141.

In another embodiment, the anti-C5 antibody, or antigen binding fragment thereof, is administered twice during the induction phase. For example, the anti-C5 antibody, or antigen binding fragment thereof, is administered on Day 1 and Day 15 of the administration cycle. In another embodiment, the anti-C5 antibody, or antigen binding fragment thereof, is administered on Day 1 and Day 22 of the administration cycle. In another embodiment, the anti-C5 antibody, or antigen binding fragment thereof, is administered twice during the induction phase at a dose of 1000 mg, 1400 mg, 1600 mg, or 2000 mg.

In another embodiment, the anti-C5 antibody, or antigen binding fragment thereof, is administered at a dose of 1400 mg on Day 1 and 1000 mg on Day 15 of the administration cycle during the induction phase. In another embodiment, the anti-C5 antibody, or antigen binding fragment thereof, is administered at a dose of 2000 mg on Day 1 and 1600 mg on Day 22 of the administration cycle during the induction phase. In another embodiment, the anti-C5 antibody, or antigen binding fragment thereof, is administered at a dose of 1600 mg on Day 1 and 1600 mg on Day 15 of the administration cycle during the induction phase. In another embodiment, the anti-C5 antibody, or antigen binding fragment thereof, is administered at a dose of 3000 mg on Day 1 of the administration cycle during the induction phase.

In another embodiment, the anti-C5 antibody, or antigen binding fragment thereof, is administered eight times during the maintenance phase at a dose of 1000 mg. For example, the anti-C5 antibody, or antigen binding fragment thereof, is administered at a dose of 1000 mg on Day 29 of the administration cycle and then every 28 days (or four weeks) thereafter during the maintenance phase, i.e., on days 57, 85, 113, 141, 169, 197, and 225 of the administration cycle.

In another embodiment, the anti-C5 antibody, or antigen binding fragment thereof, is administered five times during the maintenance phase at a dose of 1600 mg. For example, the anti-C5 antibody, or antigen binding fragment thereof, is administered at a dose of 1600 mg on Day 43 of the administration cycle and then every 42 days (or six weeks) thereafter during the maintenance phase, i.e., on days 85, 127, 169, and 211 of the administration cycle.

In another embodiment, the anti-C5 antibody, or antigen binding fragment thereof, is administered four times at a dose of 2400 mg during the maintenance phase. For example, the anti-C5 antibody, or antigen binding fragment thereof, is administered at a dose of 2400 mg on Day 29 of the administration cycle and then every 56 days (or eight weeks) thereafter during the maintenance phase, i.e., on days 85, 141, and 197 of the administration cycle.

In another embodiment, the anti-C5 antibody, or antigen binding fragment thereof, is administered three times at a dose of 5400 mg during the maintenance phase. For example, the anti-C5 antibody, or antigen binding fragment thereof, is administered at a dose of 5400 mg on Day 29 of the administration cycle and then every 84 days (or twelve weeks) thereafter during the maintenance phase, i.e., on days 113 and 197 of the administration cycle.

In another embodiment, methods of treating a human patient with PNH or aHUS are provided, wherein the methods comprise administering to the patient an effective amount of an anti-C5 antibody, or antigen binding fragment thereof, comprising (i) a heavy chain CDR1 comprising the amino acid sequence depicted in SEQ ID NO:19, (ii) a heavy chain CDR2 comprising the amino acid sequence depicted in SEQ ID NO:18, (iii) a heavy chain CDR3 comprising the amino acid sequence depicted in SEQ ID NO:3, (iv) a light chain CDR1 comprising the amino acid sequence depicted in SEQ ID NO:4, (v) a light chain CDR2 comprising the amino acid sequence depicted in SEQ ID NO:5, and (vi) a light chain CDR3 comprising the amino acid sequence depicted in SEQ ID NO:6, and wherein the methods comprise an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

In another embodiment, methods of treating a human patient with PNH or aHUS are provided, wherein the methods comprise administering to the patient an anti-C5 antibody, or antigen binding fragment thereof, comprising (i) a heavy chain CDR1 comprising the amino acid sequence depicted in SEQ ID NO:19, (ii) a heavy chain CDR2 comprising the amino acid sequence depicted in SEQ ID NO:18, (iii) a heavy chain CDR3 comprising the amino acid sequence depicted in SEQ ID NO:3, (iv) a light chain CDR1 comprising the amino acid sequence depicted in SEQ ID NO:4, (v) a light chain CDR2 comprising the amino acid sequence depicted in SEQ ID NO:5, and (vi) a light chain CDR3 comprising the amino acid sequence depicted in SEQ ID NO:6, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein the method comprises an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

In one embodiment, the anti-C5 antibody, or antigen binding fragment thereof, is administered at a dose of:
(a) 400 mg on Day 1, 400 mg on Day 8, and 600 mg on Day 15 of the administration cycle during the induction phase; and
(b) 900 mg on Days 29, 57, 85, 113, and 141 of the administration cycle during the maintenance phase.

In another embodiment, the anti-C5 antibody, or antigen binding fragment thereof, is administered at a dose of:
(a) 600 mg on Day 1 of the administration cycle and 600 mg on Day 15 of the administration cycle during the induction phase; and
(b) 900 mg on Days 29, 57, 85, 113, and 141 of the administration cycle during the maintenance phase.

In another embodiment, the anti-C5 antibody, or antigen binding fragment thereof, is administered at a dose of:
(a) 600 mg on Day 1 of the administration cycle and 900 mg on Day 15 of the administration cycle during the induction phase; and
(b) 1800 mg on Days 29, 57, 85, 113, and 141 of the administration cycle during the maintenance phase.

In another embodiment, methods of treating a human patient with PNH or aHUS are provided, wherein the methods comprise administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, wherein the method comprises an administration cycle comprising an induction phase followed by a maintenance phase, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

In another embodiment, methods of treating a human patient with PNH or aHUS are provided, wherein the methods comprise administering to the patient an anti-C5 antibody, or antigen binding fragment thereof, comprising CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

In one embodiment, the anti-C5 antibody, or antigen binding fragment thereof, is administered at a dose of:
(a) 1400 mg on Day 1 and 1000 mg on Day 15 of the administration cycle during the induction phase; and
(b) 1000 mg on Days 29, 57, 85, 113, 141, 169, 197, and 225 of the administration cycle during the maintenance phase.

In another embodiment, the anti-C5 antibody, or antigen binding fragment thereof, is administered at a dose of:
(a) 2000 mg on Day 1 and 1600 mg on Day 22 of the administration cycle during the induction phase; and
(b) 1600 mg on Days 43, 85, 127, 169, and 211 of the administration cycle during the maintenance phase.

In another embodiment, the anti-C5 antibody, or antigen binding fragment thereof, is administered at a dose of:
(a) 1600 mg on Day 1 and 1600 mg on Day 15 of the administration cycle during the induction phase; and
(b) 2400 mg on Days 29, 85, 141, and 197 of the administration cycle during the maintenance phase.

In another embodiment, the anti-C5 antibody, or antigen binding fragment thereof, is administered at a dose of:
(a) 3000 mg on Day 1 of the administration cycle during the induction phase; and
(b) 5400 mg on Days 29, 113, and 197 of the administration cycle during the maintenance phase.

In another aspect, the treatment regimens described are sufficient to maintain particular serum trough concentrations of the anti-C5 antibody, or antigen binding fragment thereof. For example, in one embodiment, the treatment maintains a serum trough concentration of the anti-C5 antibody, or antigen binding fragment thereof, of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, or 400 μg/ml or greater during the induction and/or maintenance phase. In one embodiment, the treatment maintains a serum trough concentration of the anti-C5 antibody, or antigen binding fragment thereof, of 100 μg/ml or greater during the induction and/or maintenance phase. In another embodiment, the treatment maintains a serum trough concentration of the anti-C5 antibody, or antigen binding fragment thereof, of 150 μg/ml or greater during the induction and/or maintenance phase. In another embodiment, the treatment maintains a serum trough concentration of the anti-C5 antibody, or antigen binding fragment thereof, of 200 μg/ml or greater during the induction phase and/or the maintenance phase. In another embodiment, the treatment maintains a serum trough concentration of the anti-C5 antibody, or antigen binding fragment thereof, of 250 μg/ml or greater during the induction and/or maintenance phase. In another embodiment, the treatment maintains a serum trough concentration of the anti-C5 antibody, or antigen binding fragment thereof, of 300 μg/ml or greater during the induction and/or maintenance phase. In another embodiment, the treatment maintains a serum trough concentration of the anti-C5 antibody, or antigen binding fragment thereof, of between 100 μg/ml and 200 μg/ml during the induction and/or maintenance phase. In another embodiment, the treatment maintains a serum trough concentration of the anti-C5 antibody, or antigen binding fragment thereof, of about 175 μg/ml during the induction and/or maintenance phase.

In a particular embodiment, wherein the anti-C5 antibody, or antigen binding fragment thereof, is administered at a dose of: (as) 400 mg on Day 1, 400 mg on Day 8, and 600 mg on Day 15 of the administration cycle during the induction phase; and (b) 900 mg on Days 29, 57, 85, 113, and 141 of the administration cycle during the maintenance phase, the treatment maintains a serum trough concentration of the anti-C5 antibody, or antigen binding fragment thereof, of 100 μg/ml or greater during the induction and/or maintenance phase.

In another particular embodiment, wherein the anti-C5 antibody, or antigen binding fragment thereof, is administered at a dose of: (a) 600 mg on Day 1 of the administration cycle and 900 mg on Day 15 of the administration cycle during the induction phase; and (b) 1800 mg on Days 29, 57, 85, 113, and 141 of the administration cycle during the maintenance phase, the treatment maintains a serum trough concentration of the anti-C5 antibody, or antigen binding fragment thereof, of 100 μg/ml or greater during the induction phase and 200 μg/ml or greater during the maintenance phase.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In one embodiment, the administration cycle is a period of 21 weeks. In another embodiment, the administration cycle is a period of 36 weeks. In another embodiment, the anti-C5 antibody, or antigen binding fragment thereof, is administered on a monthly basis after completion of the administration cycle, e.g., after the maintenance phase.

In another embodiment, the anti-C5 antibody, or antigen binding fragment thereof, is administered on a monthly basis for a year after completion of the administration cycle. In another embodiment, the anti-C5 antibody, or antigen binding fragment thereof, is administered on a monthly basis for two, three, four, or five years after completion of the administration cycle. In a particular embodiment, the anti-C5 antibody, or antigen binding fragment thereof, is administered on a monthly basis for up to two years after completion of the administration cycle.

In one embodiment, the anti-C5 antibody, or antigen binding fragment thereof, is administered at a dose of 900 mg or 1800 mg on a monthly basis after the maintenance phase for one, two, three, four, or five years. In another embodiment, the anti-C5 antibody, or antigen binding fragment thereof, is administered at a dose of 900 mg or 1800 mg on a monthly basis after the maintenance phase for up to two years.

In another embodiment, the anti-C5 antibody, or antigen binding fragment thereof, is administered at a dose of 1000 mg every four weeks, 1600 mg every six weeks, 2400 mg every eight weeks, or 5400 mg every twelve weeks after the maintenance phase.

In another embodiment, the “maintenance phase” utilizing the maintenance dosage and dosing interval are extended beyond the clinical trial for 1 year, 2 years, 3 years, 4 years, 5 years, 6 years, 7 years, 8 years, 9 years 10 years, 15 years or more or for the lifetime of the patient.

The anti-C5 antibodies, or antigen binding fragments thereof, can be administered to a patient by any suitable means. In one embodiment, the antibodies are formulated for intravenous administration.

The efficacy of the treatment methods provided herein can be assessed using any suitable means. For example, for aHUS patient, the treatment produces at least one therapeutic effect selected from the group consisting of a reduction or cessation in or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)).

In another embodiment, for a PNH patient, the treatment produces at least one therapeutic effect selected from the group consisting of a reduction or cessation in fatigue, abdominal pain, dyspnea, dysphagia, chest pain, and erectile dysfunction. In another embodiment, the treatment results in terminal complement inhibition. In another embodiment, the treatment results in a reduction of hemolysis as assessed by lactate dehydrogenase (LDH) levels. In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer. In another embodiment, the treatment produces a reduction in the need for blood transfusions. In another embodiment, the treatment produces a reduction in major adverse vascular events (MAVEs). In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP). In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale.

In a particular embodiment, lactate dehydrogenase (LDH) levels are used to evaluate responsiveness to a therapy (e.g., a reduction of hemolysis as assessed by lactate dehydrogenase (LDH) levels is indicative of an improvement in at least one sign of PNH). In one embodiment, patients treated according to the disclosed methods experience reductions in LDH levels to near normal levels or to within 10%, or within 20% above what is considered the normal level (e.g., within 105-333 IU/L (international units per liter). In one embodiment, the patient's LDH levels are ≥1.5 fold above the upper limit of normal (LDH≥1.5×ULN) prior to initiating treatment. In another embodiment, the patient's LDH levels are about 6×ULN prior to initiating treatment. In another embodiment, the patient's LDH levels are about 7×ULN prior to initiating treatment.

In one embodiment, patients treated according to the disclosed methods experience reductions in LDH levels to within normal levels or to within 10%, 20%, 30%, 40% or within 50% below what is considered the upper limit of normal level (e.g., within 105-333 IU/L (international units per liter). In one embodiment, the patient's LDH levels are ≥1.5 fold above the upper limit of normal (LDH≥1.5×ULN) prior to initiating treatment. In another embodiment, patients treated according to the disclosed methods experience a continued (e.g., sustained) reduction to below the ULN in LDH levels compared to baseline for 4, 5, 6, 7, 8, 9, 10, 11, 12, 13, 14, 15, 16, 17, 18, 19, 20, 21, 22, 23, 24, 25, 26 weeks or more after initiating treatment.

In another embodiment, LDH levels rapidly decrease after initiating treatment (e.g., within 1, 2, 3, 4, 5, 6, 7, 8, or 9 days after initiating treatment). In one embodiment, LDH levels rapidly decrease within 8 days of initiating treatment. In another embodiment, LDH levels decrease by about 3 fold within 8 days of initiating treatment. In another particular embodiment, LDH levels decrease by about 3.5 fold within 8 days of initiating treatment.

In another embodiment, LDH levels decrease by about 4, 5, 6, 7, 8, or 9 fold, four weeks after initiating treatment. In a particular embodiment, LDH levels decrease by about 6 fold, four weeks after initiating treatment. In a particular embodiment, LDH levels decrease by about 7 fold, four weeks after initiating treatment. In a particular embodiment, LDH levels decrease by about 8 fold, four weeks after initiating treatment. In another embodiment, LDH levels decrease by about 4, 5, 6, 7, 8, 9, or 10 fold, six weeks after initiating treatment. In a particular embodiment, LDH levels decrease by about 8 or 9 fold, four weeks after initiating treatment.

In another embodiments, patients treated according to the disclosed methods experience reductions in LDH levels by about 20%, 30%, 40%, 50%, 60%, 70%, 80% or more compared to no treatment. In one embodiment, there is about an 80%, 81%, 82% 83%, 84%, 85%, 86%, 87%, 88%, 89%, or 90% percent change from baseline four weeks after initiating treatment. In a particular embodiment, there is about an 84% percent change from baseline four weeks after initiating treatment. In another particular embodiment, there is about an 85% percent change from baseline four weeks after initiating treatment. In another particular embodiment, there is about an 88% percent change from baseline four weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline six weeks after initiating treatment. In a particular embodiment, there is about an 89% percent change from baseline six weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline about eight weeks after initiating treatment. In a particular embodiment, there is about an 84% percent change from baseline about eight weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline about sixteen weeks after initiating treatment. In a particular embodiment, there is about an 86% percent change from baseline about sixteen weeks after initiating treatment.

In another embodiment, patients treated according to the disclosed methods experience a continued (e.g., sustained) reduction in LDH levels compared to baseline for 4, 5, 6, 7, 8, 9, 10, 11, 12, 13, 14, 15, 16, 17, 18, 19, 20, 21, 22, 23, 24, 25, 26 weeks or more after initiating treatment. In one embodiment, an administration cycle according to the treatment methods described herein comprises a period of 21 weeks. After the administration cycle, the patient can receive additional doses of the anti-C5 antibody, or antigen binding fragment thereof. In one embodiment, the anti-C5 antibody, or antigen binding fragment thereof, is administered on a monthly basis after the maintenance phase. In another embodiment, the anti-C5 antibody, or antigen binding fragment thereof, is administered at a dose of 900 mg or 1800 mg on a monthly basis. In another embodiment, the anti-C5 antibody, or antigen binding fragment thereof, is administered after administration cycle for up to two years.

In another aspect, an anti-C5 antibody, or antigen binding fragment thereof, is provided, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration in a cycle comprising an induction phase followed by a maintenance phase, wherein:

(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and

(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle. In one embodiment, the antibody is determined to be safe, tolerable and sufficiently non-immunogenic after multiple IV doses for use in PNH or aHUS patients.

In another aspect, an anti-C5 antibody, or antigen binding fragment thereof, is provided, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration in a cycle comprising an induction phase followed by a maintenance phase, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

In one embodiment, the antibody is determined to be safe, tolerable and sufficiently non-immunogenic after multiple IV doses for use in PNH or aHUS patients.

Further provided are kits that include a pharmaceutical composition containing an anti-C5 antibody, or antigen binding fragment thereof, such as antibody BNJ441, and a pharmaceutically-acceptable carrier, in a therapeutically effective amount adapted for use in the methods described herein.

In one embodiment, the kit comprises: (a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody, or antigen binding fragment thereof, in any of the methods described herein.

In another embodiment, the kit comprises:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, in the methods described herein.

In a particular embodiment, the anti-C5 antibody, or antigen binding fragment thereof, is administered at a dose of 1400 mg on Day 1 and 1000 mg on Day 15 of the administration cycle during the induction phase and 1000 mg on Days 29, 57, 85, 113, 141, 169, 197, and 225 of the administration cycle during the maintenance phase. In another embodiment, the anti-C5 antibody, or antigen binding fragment thereof, is administered at a dose of: 2000 mg on Day 1 and 1600 mg on Day 22 of the administration cycle during the induction phase; and 1600 mg on Days 43, 85, 127, 169, and 211 of the administration cycle during the maintenance phase. In yet another embodiment, the anti-C5 antibody, or antigen binding fragment thereof, is administered at a dose of: 1600 mg on Day 1 and 1600 mg on Day 15 of the administration cycle during the induction phase; and 2400 mg on Days 29, 85, 141, and 197 of the administration cycle during the maintenance phase. In yet another embodiment, the anti-C5 antibody, or antigen binding fragment thereof, is administered at a dose of: 3000 mg on Day 1 of the administration cycle during the induction phase; and 5400 mg on Days 29, 113, and 197 of the administration cycle during the maintenance phase.

### BRIEF DESCRIPTION OF THE DRAWINGS

FIG. 1 is a schematic depicting the study design for the open-label, intrapatient, dose-escalation study in PNH patients (described below in Section 1, Examples 1-5).

FIG. 2 is the raw LDH data for individual patients in Cohorts 1a and 1b after treatment with ALXN1210, as well as the raw LDH data for PNH patients after treatment with eculizumab (for comparative purposes).

FIG. 3 is the raw mean LDH data for patients in Cohorts 1a and 1b after treatment with ALXN1210, as well as the raw mean LDH data for PNH patients after treatment with eculizumab (for comparative purposes).

FIG. 4 is a graph which depicts the mean LDH data for the patients in Cohorts 1a and 1b (treated with ALXN1210), compared to patients treated with eculizumab or a placebo.

FIG. 5 is the raw LDH percentage change from baseline data for patients in Cohorts 1a and 1b (after treatment with ALXN1210), as well as the raw LDH percentage change from baseline data for PNH patients after treatment with eculizumab (for comparative purposes).

FIG. 6 is a graph which depicts the LDH percentage change from baseline for the patients in Cohorts 1a and 1b (treated with ALXN1210), compared to the LDH percentage change from baseline for patients treated with eculizumab or a placebo.

FIG. 7 is a graph which depicts mean (SD) LDH values over time by Cohort compared to baseline.

FIG. 8 is a graph which depicts the mean LDH over time for patients in Cohorts 1 and 2 (treated with ALXN1210), compared to the LDH over time for patients treated with eculizumab or a placebo.

FIG. 9 is a graph which depicts the mean percent change in LDH over time for patients in Cohorts 1 and 2 (treated with ALXN1210), compared to the mean percent change in LDH over time for patients treated with eculizumab or a placebo.

FIGS. 10A-10B set forth the raw mean, median, and minimum/maximum percentage change in LDH levels from baseline data for patients in Cohorts 1 and 2 after treatment with ALXN1210 from Week 1 through Week 8 (FIG. 10A) and from Week 12 through Week 24 (FIG. 10B).

FIGS. 11A-11B set forth the raw mean LDH normalization data for patients in Cohorts 1 and 2 after treatment with ALXN1210 from Week 1 through Week 8 (FIG. 11A) and from Week 12 through Week 24 (FIG. 11B).

FIGS. 12A-D display preliminary serum PK, free and total C5 concentrations, and LDH activity 20 following multiple dose administration in PNH patients.

FIGS. 13A-13B set forth the preliminary mean (range) ALXN1210 concentrations and free and total C5 concentrations at baseline and end of infusion.

FIGS. 14A-14B set forth the preliminary mean (range) ALXN1210 concentrations and free and total C5 concentrations at baseline and pre-dose.

FIGS. 15A-15B show preliminary mean (range) pre-dose PK, LDH, free C5, percent change from baseline in free C5, and total C5 at additional time points. “% CFB” refers to percent change from baseline in the context of free C5 measurements.

FIG. 16 sets forth the free C5 and hemolytic assay data.

FIG. 17 are bar graphs which compare 900 mg and 1800 mg (Q4W) dosages of ALXN1210 on FACIT-Fatigue Score.

FIG. 18 is a schematic depicting the design of the phase 2, open-label, multiple ascending dose study in PNH patients (described below in Section 2, Examples 6-8).

FIG. 19 is a graph which depicts the mean LDH over time for patients in Cohorts 1, 2, and 3 (treated with ALXN1210), compared to the LDH over time for patients treated with eculizumab or a placebo.

FIG. 20 is a graph which depicts the mean percent change in LDH over time for patients in Cohorts 1, 2, and 3 (treated with ALXN1210), compared to the mean percent change in LDH over time for patients treated with eculizumab or a placebo.

FIGS. 21A-21B set forth the raw mean, median, and minimum/maximum percentage change in LDH levels from baseline data for patients in Cohorts 1, 2, and 3 after treatment with ALXN1210 from Week 1 through Week 4 (FIG. 21A) and from Week 6 through Week 16 (FIG. 21B).

FIG. 22 sets forth the raw mean LDH normalization data for patients in Cohorts 1, 2, and 3 after treatment with ALXN1210 through Day 113.

FIGS. 23A-D display preliminary serum PK, free and total C5 concentrations, and LDH activity following multiple dose administration in PNH patients.

FIG. 24 sets forth the preliminary mean (range) ALXN1210 concentrations and free and Total C5 concentrations at baseline and end of infusion.

FIG. 25 sets forth the preliminary mean (range) ALXN1210 concentrations and free and Total C5 concentrations at baseline and predose.

FIG. 26 is a summary of the trough pharmacokinetic and pharmacodynamic data by cohort through Day 113 (Cohort 4), Day 141 (Cohort 3) and Day 169 (Cohorts 1 and 2).

FIG. 27 is a graph depicting the change in LDH levels for all four Cohorts during treatment with ALXN1210.

FIG. 28 is a graph depicting the change in LDH levels for Cohort 1 (Q4W) during treatment with ALXN1210.

FIG. 29 is a graph depicting the change in LDH levels for Cohort 2 (Q6W) during treatment with ALXN1210.

FIG. 30 is a graph depicting the change in LDH levels for Cohort 3 (Q8W) during treatment with ALXN1210.

FIG. 31 is a graph depicting the change in LDH levels for Cohort 4 (Q12W) during treatment with ALXN1210.

FIG. 32 depicts the change in hemoglobin during treatment with ALXN1210 (including transfused patients).

FIG. 33 depicts the change in hemoglobin during treatment with ALXN1210 (excluding the five transfused patients).

FIG. 34 is a summary of the FACIT-Fatigue scores by cohort at Day 57.

FIG. 35 is a summary of the FACIT-Fatigue scores by cohort at Day 113.

FIG. 36 is a summary of the FACIT-Fatigue scores by cohort at Day 127.

FIG. 37 is a summary of the FACIT-Fatigue scores by cohort at Day 197.

### DETAILED DESCRIPTION

### I. Definitions

As used herein, the term “subject” or “patient” is a human patient (e.g., a patient having Paroxysmal Nocturnal Hemoglobinuria (PNH)) or atypical hemolytic uremic syndrome (aHUS)). PNH and aHUS, are both ultra-rare disorders driven by chronic uncontrolled complement activation. In each case, ongoing complement dysregulation leads to increased activation of C5 systemically with consequent terminal complement activation, resulting in the devastating clinical manifestations of these disorders. Patients with PNH or aHUS are at risk of substantial morbidity and mortality.

Paroxysmal nocturnal hemoglobinuria is an acquired hemolytic disorder that occurs most frequently in adults (Brodsky R A., Blood. 2015; 126:2459-65). The disease begins with the clonal expansion of a hematopoietic stem cell that has acquired a somatic mutation in the PIGA gene (Brodsky R A., Blood. 2014; 124:2804-1). Consequently, PNH blood cells lack the glycophosphatidylinositol (GPI) anchor protein and are deficient in the membrane-bound complement inhibitory proteins CD55 and CD59. In the absence of CD55, there is increased deposition of complement protein C3 cleavage products on blood cell membrane surfaces, in turn leading to cleavage of C5 into C5a and C5b. The pathology and clinical presentations in patients with PNH are driven by uncontrolled terminal complement activation.

C5a is a potent anaphylatoxin, chemotactic factor, and cell-activating molecule that mediates multiple pro-inflammatory and pro-thrombotic activities (Matis L A, et al., Nat. Med. 1995; 1:839-42; Prodinger et al., Complement. In: Paul W E, editor. Fundamental immunology (4th ed). Philadelphia: Lippincott-Raven Publishers; 1999. p. 967-95). C5b recruits the terminal complement components C6, C7, C8, and C9 to form the pro-inflammatory, pro-thrombotic cytolytic pore molecule C5b-9, a process that under normal circumstances would be blocked on the red blood cell (RBC) membrane by CD59. In patients with PNH, however, these final steps proceed unchecked, culminating in hemolysis and the release of free hemoglobin, as well as platelet activation (Hill, et al., Blood 2013; 121:4985-96). The signs and symptoms of PNH can be attributed to chronic, uncontrolled complement C5 cleavage, and release of C5a and C5b-9 leading to RBC hemolysis, which together result in (Hill, et al., Blood 2013; 121:4985-96; Brodsky R A., Blood. 2014; 124:2804-1):
Release of intracellular free hemoglobin and lactate dehydrogenase (LDH) into circulation as a direct consequence of hemolysis.
Irreversible binding to and inactivation of nitric oxide (NO) by hemoglobin, and inhibition of NO synthesis.
Vasoconstriction and tissue-bed ischemia due to absence of vasodilatory NO, as well as possible microthrombi manifesting as abdominal pain, dysphagia, and erectile dysfunction.
Platelet activation.
A pro-inflammatory and prothrombotic state.

A substantial proportion of patients with PNH experience renal dysfunction and pulmonary hypertension (Hillmen, et al., Am J Hematol. 2010; 85:553-9. [erratum in Am J Hematol. 2010; 85:911.]; Hill, et al., Br. J Haematol. 2012; 158:409-14.; Hill, et al., Blood 2013; 121:4985-96). Patients also experience venous or arterial thrombosis in diverse sites, including the abdomen or central nervous system (Brodsky R A., Blood. 2014; 124:2804-1).

The pathology and clinical presentations of patients with aHUS are also driven by terminal complement activation. More specifically, activation of C5 and dysregulation of complement activation lead to endothelial damage, platelet consumption, and thrombotic microangiopathic (TMA) events, characterized by thrombocytopenia, mechanical intravascular hemolysis, and kidney injury. Importantly, approximately 20% of patients experience extra-renal manifestations of disease as well, including central nervous system, cardiac, gastrointestinal, distal extremities, and severe systemic organ involvement (Loirat, et al., Orphanet. J. Rare Dis. 2011; 6:60). Symptoms of aHUS are well-known to those of skill in the art of rare disease or kidney disease medicine and include, e.g., severe hypertension, proteinuria, uremia, lethargy/fatigue, irritability, thrombocytopenia, microangiopathic hemolytic anemia, and renal function impairment (e.g., acute renal failure).

aHUS can be genetic, acquired, or idiopathic. aHUS can be considered genetic when two or more (e.g., three, four, five, or six or more) members of the same family are affected by the disease at least six months apart and exposure to a common triggering agent has been excluded, or when one or more aHUS-associated gene mutations (e.g., one or more mutations in CFH, MCP/CD46, CFB, or CFI) are identified in a subject. For example, a subject can have CFH-associated aHUS, CFB-associated aHUS, CFI-associated aHUS, or MCP-associated aHUS. Up to 30% of genetic aHUS is associated with mutations in CFH, 12% with mutations in MCP, 5-10% with mutations in CFI, and less than 2% with mutations in CFB. Genetic aHUS can be multiplex (i.e., familial; two or more affected family members) or simplex (i.e., a single occurrence in a family). aHUS can be considered acquired when an underlying environmental factor (e.g., a drug, systemic disease, or viral or bacterial agents that do not result in Shiga-like exotoxins) or trigger can be identified. aHUS can be considered idiopathic when no trigger (genetic or environmental) is evident.

Laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668). Blood concentrations of C3 and C4 can also be used as a measure of complement activation or dysregulation. In addition, a subject's condition can be further characterized by identifying the subject as harboring one or more mutations in a gene associated with aHUS such as CFI, CFB, CFH, or MCP (supra). Suitable methods for detecting a mutation in a gene include, e.g., DNA sequencing and nucleic acid array techniques. See, e.g., Breslin et al. (2006) Clin Am Soc Nephrol 1:88-99 and Goicoechea de Jorge et al. (2007) Proc Natl Acad Sci USA 104:240-245.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of PNH (e.g., fatigue, abdominal pain, dyspnea, dysphagia, chest pain, or erectile dysfunction) or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)).

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In one example, an “effective amount” is the amount of anti-C5 antibody, or antigen binding fragment thereof, clinically proven to alleviate at least one symptom of PNH (e.g., fatigue, abdominal pain, dyspnea, dysphagia, chest pain, or erectile dysfunction) or at least one symptom of aHUS (e.g., severe hypertension, proteinuria, uremia, lethargy/fatigue, irritability, thrombocytopenia, microangiopathic hemolytic anemia, and renal function impairment (e.g., acute renal failure)). An effective amount can be administered in one or more administrations.

As used herein, the terms “induction” and “induction phase” are used interchangeably and refer to the first phase of treatment in the clinical trial.

As used herein, the terms “maintenance” and “maintenance phase” are used interchangeably and refer to the second phase of treatment in the clinical trial. In certain embodiments, treatment is continued as long as clinical benefit is observed or until unmanageable toxicity or disease progression occurs.

As used herein, the “maintenance phase” utilizing the maintenance dosage and dosing interval are extended beyond the clinical trial for 1 year, 2 years, 3 years, 4 years, 5 years, 6 years, 7 years, 8 years, 9 years 10 years, 15 years or more or for the lifetime of the patient.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-C5 antibody, or antigen binding fragment thereof).

As used herein, the term “serum trough level” refers to the lowest level that the agent (e.g., the anti-C5 antibody, or antigen binding fragment thereof,) or medicine is present in the serum. In contrast, a “peak serum level”, refers to the highest level of the agent in the serum. The “average serum level”, refers to the mean level of the agent in the serum over time.

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

### II. Anti-C5 Antibodies

The anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

An exemplary anti-C5 antibody is antibody BNJ441 comprising heavy and light chains having the sequences shown in SEQ ID NOs:14 and 11, respectively, or antigen binding fragments and variants thereof. BNJ441 (also known as ALXN1210) is described in PCT/US2015/019225 and U.S. Pat. No. 9,079,949, the teachings or which are hereby incorporated by reference. BNJ441 is a humanized monoclonal antibody that is structurally related to eculizumab (Soliris®). BNJ441 was derived through minimal targeted engineering of eculizumab by introducing four unique amino acid substitutions into the heavy chain, with the objective of enhancing the duration of terminal complement inhibition, while maintaining key eculizumab attributes. BNJ441 and eculizumab share over 99% primary sequence identity and have very similar pharmacology. BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389 1401] exemplifies the identification of CDR boundaries according to Kabat and Chothia definitions.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22, and 23, respectively, and light chain CDR1, CDR2 and CDR3 domains having the sequences set forth in SEQ ID NOs: 24, 25, and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30, and 31, respectively, and light chain CDR1, CDR2 and CDR3 domains having the sequences set forth in SEQ ID NOs: 32, 33, and 34, respectively. In another embodiment, the antibody comprises the VH region of the 8110 antibody having the sequence set forth in SEQ ID NO: 35, and the VL region of the 8110 antibody having the sequence set forth in SEQ ID NO: 36.

Another exemplary anti-C5 antibody is the 305LO5 antibody described in US2016/0176954A1. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs: 37, 38, and 39, respectively, and light chain CDR1, CDR2 and CDR3 domains having the sequences set forth in SEQ ID NOs: 40, 41, and 42, respectively. In another embodiment, the antibody comprises the VH region of the 305LO5 antibody having the sequence set forth in SEQ ID NO: 43, and the VL region of the 305LO5 antibody having the sequence set forth in SEQ ID NO: 44.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q3111, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable an anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody, or antigen binding fragment thereof, on complement activation. In order to determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microas say wells, which are coated with a capture reagent (e.g., an antibody that binds to one or more components of the TCC). The TCC present in the activated samples bind to the monoclonal antibodies coating the surface of the microas say wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55, or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90, or 95) % of the complement inhibitory activity of eculizumab in a hemolytic assay or CH50eq assay. An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### III. Compositions

Also, provided herein are compositions comprising an anti-C5 antibody, or antigen binding fragment thereof. In one embodiment, the composition comprises an anti-C5 antibody comprising the CDR1, CDR2 and CDR3 domains in a heavy chain variable region having the sequence set forth in SEQ ID NO:12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs:14 and 11, respectively. In another embodiment, the anti-C5 antibody comprises heavy and light chains having the sequences shown in SEQ ID NOs:20 and 11, respectively.

The compositions can be formulated as a pharmaceutical solution, e.g., for administration to a subject for the treatment or prevention of a complement-associated disorder. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 11/2 years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

### IV. Outcomes

Provided herein are methods for treating PNH in a patient comprising administering to the patient an anti-C5 antibody. Symptoms of PNH include, but are not limited to, fatigue (e.g., tiredness, difficulty performing daily activities, trouble concentrating, dizziness, weakness), pain (e.g., stomach pain, leg pain or swelling, chest pain, back pain), dark-colored urine, shortness of breath, difficulty swallowing, yellowing of the skin and/or eyes, erectile dysfunction, blood clots, kidney disease, damage to organs, stroke, or heart attack. Patients treated according to the methods disclosed herein preferably experience improvement in at least one sign of PNH. For example, the treatment may produce at least one therapeutic effect selected from the group consisting of a reduction or cessation in fatigue, abdominal pain, dyspnea, dysphagia, chest pain, and erectile dysfunction.

Also provided herein are methods for treating aHUS in a patient comprising administering to the patient an anti-C5 antibody. Symptoms of aHUS include, but are not limited to, severe hypertension, proteinuria, uremia, lethargy/fatigue, irritability, thrombocytopenia, microangiopathic hemolytic anemia, and renal function impairment (e.g., acute renal failure). Patients treated according to the methods disclosed herein preferably experience improvement in at least one sign of aHUS. For example, the treatment may produce at least one therapeutic effect selected from the group consisting of a reduction or cessation in severe hypertension, proteinuria, uremia, lethargy/fatigue, irritability, thrombocytopenia, microangiopathic hemolytic anemia, and renal function impairment (e.g., acute renal failure).

In another embodiment, improvement is measured by terminal complement inhibition.

In another embodiment, lactate dehydrogenase (LDH) levels can be used to evaluate responsiveness to a therapy (e.g., a reduction of hemolysis as assessed by lactate dehydrogenase (LDH) levels is indicative of an improvement in at least one sign of PNH).

LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011). LDH concentration obtained at baseline and then serially throughout a treatment period, is an important measure of hemolysis. Baseline levels of cell-free plasma hemoglobin are highly elevated in patients with PNH with LDH≥1.5-fold above the upper limit of normal (LDH≥1.5×ULN), with a significant correlation between LDH and cell-free plasma hemoglobin (Hillmen, P. et al., N. Engl. J. Med., 355:1233-43, 2006). The normal LDH value range is 105-333 IU/L (international units per liter).

LDH levels can be measured using any suitable test or assay, such as those described by Ferri F F, ed. Ferri's Clinical Advisor 2014. Philadelphia: Pa: Elsevier Mosby; 2014: Section IV—Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are known in the art and can be readily adapted to obtain a sample that is compatible with the method utilized.

In one embodiment, patients treated according to the disclosed methods experience reductions in LDH levels to near normal levels or to within 10%, or within 20% above what is considered the normal level (e.g., within 105-333 IU/L (international units per liter). In one embodiment, the patient's LDH levels are ≥1.5 fold above the upper limit of normal (LDH≥1.5×ULN) prior to initiating treatment. In another embodiment, the patient's LDH levels are about 6×ULN prior to initiating treatment. In another embodiment, the patient's LDH levels are about 7×ULN prior to initiating treatment.

In another embodiment, LDH levels rapidly decrease after initiating treatment (e.g., within 1, 2, 3, 4, 5, 6, 7, 8, or 9 days after initiating treatment). In one embodiment, LDH levels rapidly decrease within 8 days of initiating treatment. For example, in one embodiment, LDH levels decrease by about 2, 3, 4, or 5 fold within 8 days of initiating treatment. In a particular embodiment, LDH levels decrease by about 3 fold within 8 days of initiating treatment. In another particular embodiment, LDH levels decrease by about 3.5 fold within 8 days of initiating treatment.

In one embodiment, patients treated according to the disclosed methods experience reductions in LDH levels to within normal levels or to within 10%, 20%, 30%, 40% or within 50% below what is considered the upper limit of normal level (e.g., within 105-333 IU/L (international units per liter). In one embodiment, the patient's LDH levels are ≥1.5 fold above the upper limit of normal (LDH≥1.5×ULN) prior to initiating treatment. In another embodiment, patients treated according to the disclosed methods experience a continued (e.g., sustained) reduction to below the ULN in LDH levels compared to baseline for 4, 5, 6, 7, 8, 9, 10, 11, 12, 13, 14, 15, 16, 17, 18, 19, 20, 21, 22, 23, 24, 25, 26 weeks or more after initiating treatment.

In another embodiment, LDH levels decrease by about 4, 5, 6, 7, 8, or 9 fold, four weeks after initiating treatment. In a particular embodiment, LDH levels decrease by about 6 fold, four weeks after initiating treatment. In a particular embodiment, LDH levels decrease by about 7 fold, four weeks after initiating treatment. In a particular embodiment, LDH levels decrease by about 8 fold, four weeks after initiating treatment. In another embodiment, LDH levels decrease by about 4, 5, 6, 7, 8, 9, or 10 fold, six weeks after initiating treatment. In a particular embodiment, LDH levels decrease by about 8 or 9 fold, four weeks after initiating treatment.

In other embodiments, patients treated according to the disclosed methods experience reductions in LDH levels by about 20%, 30%, 40%, 50%, 60%, 70%, 80% or more compared to no treatment. In one embodiment, there is about an 80%, 81%, 82% 83%, 84%, 85%, 86%, 87%, 88%, 89%, or 90% percent change from baseline four weeks after initiating treatment. In a particular embodiment, there is about an 84% percent change from baseline four weeks after initiating treatment. In another particular embodiment, there is about an 85% percent change from baseline four weeks after initiating treatment. In another particular embodiment, there is about an 88% percent change from baseline four weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline six weeks after initiating treatment. In a particular embodiment, there is about an 89% percent change from baseline six weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline about eight weeks after initiating treatment. In a particular embodiment, there is about an 84% percent change from baseline about eight weeks after initiating treatment.

In another embodiment, there is about an 80%, 81%, 82% 83%, 84%, 85%, 86%, 87%, 88%, 89%, 90%, 91%, 92%, 93%, 94%, or 95% percent change from baseline about sixteen weeks after initiating treatment. In a particular embodiment, there is about an 86% percent change from baseline about eight weeks after initiating treatment.

In another embodiment, patients treated according to the disclosed methods experience a continued (e.g., sustained) reduction in LDH levels compared to baseline for 4, 5, 6, 7, 8, 9, 10, 11, 12, 13, 14, 15, 16, 17, 18, 19, 20, 21, 22, 23, 24, 25, 26 weeks or more after initiating treatment.

In another embodiment, the treatment produces a shift toward normal levels of a hemolysis-related hematologic biomarker selected from the group consisting of free hemoglobin, haptoglobin, reticulocyte count, PNH red blood cell (RBC) clone and D-dimer.

In another embodiment, the treatment produces a reduction in the need for blood transfusions.

In another embodiment, the treatment produces a reduction in major adverse vascular events (MAVEs) (e.g., thrombophlebitis/deep vein thrombosis, pulmonary embolus, myocardial infarction, transient ischemic attack, unstable angina, renal vein thrombosis/renal artery thrombosis/glomerular thrombosis, renal infarction, acute peripheral vascular occlusion, mesenteric/visceral vein/arterial thrombosis or infarction, hepatic/portal vein thrombosis, cerebral arterial occlusion/cerebrovascular accident, cerebral venous occlusion, renal arterial thrombosis, or multi-infarct dementia), as described in further detail in the Examples.

In another embodiment, the treatment produces a shift toward normal levels of a chronic disease associated biomarker selected from the group consisting estimated glomerular filtration rate (eGFR) and spot urine:albumin:creatinine and plasma brain natriuretic peptide (BNP).

In another embodiment, the treatment produces a change from baseline in quality of life as assessed via the Functional Assessment of Chronic Illness Therapy (FACIT)-Fatigue Scale, version 4 and the European Organisation for Research and Treatment of Cancer, Quality of Life Questionnaire-Core 30 Scale, and described in further detail in the Examples.

### V. Kits and Unit Dosage Forms

Also provided herein are kits which include a pharmaceutical composition containing an anti-C5 antibody, or antigen binding fragment thereof, such as antibody BNJ441 or BNJ421, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having PNH or aHUS. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody, or antigen binding fragment thereof.

In one embodiment, the present invention provides a kit for treating PNH or aHUS in a human patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In another embodiment, the present invention provides a kit for treating PNH or aHUS in a human patient, the kit comprising:

(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and

(b) instructions for using the anti-C5 antibody, or antigen binding fragment thereof, according to any of the methods described herein.

In one embodiment, the kit comprises a dose of an anti-C5 antibody, or antigen binding fragment thereof, wherein the anti-C5 antibody, or antigen binding fragment thereof, is administered at a dose of:
(a) 1400 mg on Day 1 and 1000 mg on Day 15 of the administration cycle during the induction phase; and
(b) 1000 mg on Days 29, 57, 85, 113, 141, 169, 197, and 225 of the administration cycle during the maintenance phase.

In another embodiment, the kit comprises a dose of an anti-C5 antibody, or antigen binding fragment thereof, wherein the anti-C5 antibody, or antigen binding fragment thereof, is administered at a dose of:
(a) 2000 mg on Day 1 and 1600 mg on Day 22 of the administration cycle during the induction phase; and
(b) 1600 mg on Days 43, 85, 127, 169, and 211 of the administration cycle during the maintenance phase.

In another embodiment, the kit comprises a dose of an anti-C5 antibody, or antigen binding fragment thereof, wherein the anti-C5 antibody, or antigen binding fragment thereof, is administered at a dose of:
(a) 1600 mg on Day 1 and 1600 mg on Day 15 of the administration cycle during the induction phase; and
(b) 2400 mg on Days 29, 85, 141, and 197 of the administration cycle during the maintenance phase.

In another embodiment, the kit comprises a dose of an anti-C5 antibody, or antigen binding fragment thereof, wherein the anti-C5 antibody, or antigen binding fragment thereof, is administered at a dose of:
(a) 3000 mg on Day 1 of the administration cycle during the induction phase; and
(b) 5400 mg on Days 29, 113, and 197 of the administration cycle during the maintenance phase.

The following examples are merely illustrative and should not be construed as limiting the scope of this disclosure in any way as many variations and equivalents will become apparent to those skilled in the art upon reading the present disclosure.

The contents of all references, Genbank entries, patents and published patent applications cited throughout this application are expressly incorporated herein by reference.

### EXAMPLES SECTION 1: OPEN-LABEL, INTRAPATIENT, DOSE-ESCALATION STUDY IN PNH PATIENTS (EXAMPLES 1-5)

### Example 1: Overview of Study

An open-label, multiple-dose, multi-center intrapatient dose-escalation study is conducted to explore the safety, tolerability, efficacy pharmacokinetics (PK)/pharmacodynamics (PD), and immunogenicity of antibody BNJ441 (also known as ALXN1210) in patients with PNH who have not previously been treated with a complement inhibitor.

1. Objectives

The primary objective of the study is to evaluate the safety, tolerability, and efficacy of multiple intravenous (IV) doses of ALXN1210 administered to complement inhibitor treatment-naïve patients with PNH.

Secondary objectives include characterizing the PK and PD effects of multiple IV doses of ALXN1210 administered to complement inhibitor treatment-naïve patients with PNH and investigating the immunogenicity of ALXN1210 administered IV to complement inhibitor treatment-naïve patients with PNH.

2. Study Design

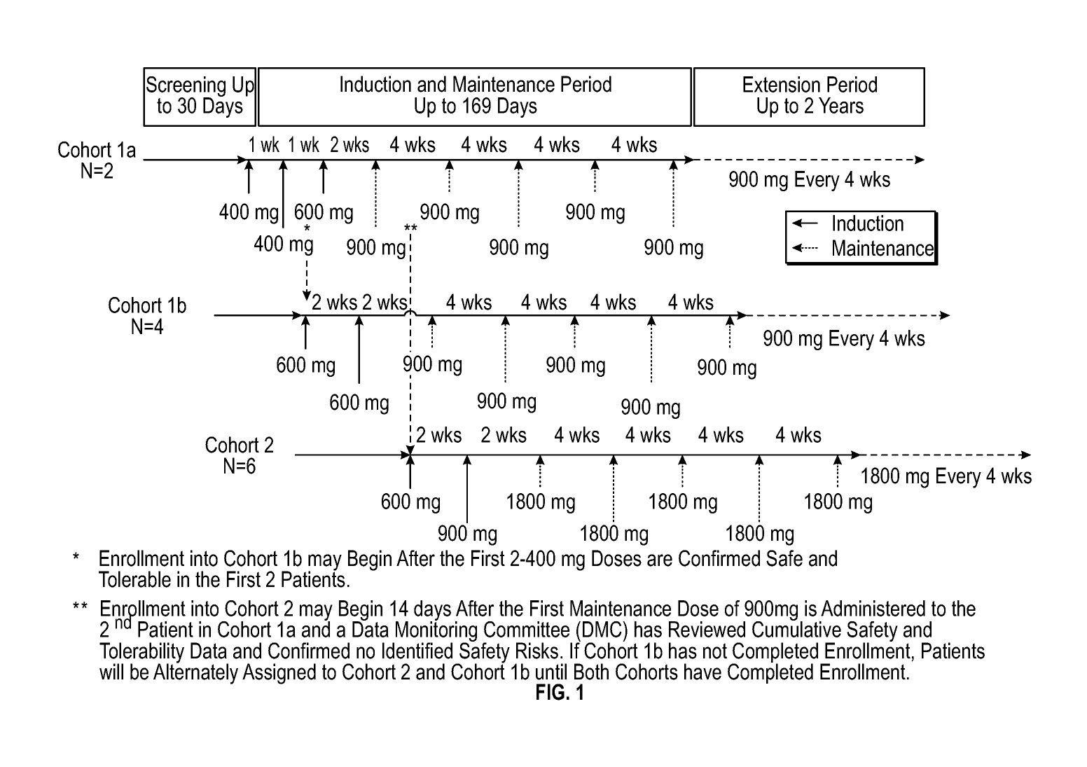

The overall study design, treatments and study durations are depicted in FIG. 1. A total of 2 treatment cohorts and 12 patients (6 per cohort) are enrolled. All patients are screened for study eligibility after providing written informed consent to participate. Patients who fail to meet any of the eligibility criteria can be rescreened once. Dosing information for each cohort is provided in Table 1.

TABLE 1
Dosing Schedule

| Cohort | Induction | Maintenance |
|---|---|---|
| 1a | 400 mg on Day 1 | 900 mg on Day 29 and then every 28 |
| | 400 mg on Day 8 | days (Days 57, 85, 113, 141) for a |
| | 600 mg on Day 15 | total of 5 doses |
| 1b | 600 mg on Day 1¹ | 900 mg on Day 29 and then every 28 |
| | 600 mg on Day 15 | days (Days 57, 85, 113, 141) for a |
| | | total of 5 doses |
| 2 | 600 mg on Day 1² | 1800 mg on Day 29 and then every 28 |
| | 900 mg on Day 15 | days (Days 57, 85, 113, 141) for a |
| | | total of 5 doses³ |

¹After safety review of Cohort 1a post second 400 mg dose
²After Data Monitoring Committee (DMC) review of Cohort 1a after the fourth dose (first 900 mg dose) + 14 Days
³Based on confirmation of PK; dose adjustments if necessary can be made.

Patients enrolled in Cohort 1a receive induction doses of ALXN1210 of 400 mg on Day 1, 400 mg on Day 8 and 600 mg on Day 15. On Day 29, they receive the first of 5 monthly maintenance doses of 900 mg of ALXN1210.

After review of safety data from the first two 400 mg doses administered to patients in Cohort 1a, patients are enrolled into Cohort 1b. Patients in Cohort 1b receive 2 induction doses of 600 mg of ALXN1210, separated by 2 weeks, and followed by 5 monthly maintenance doses of 900 mg of ALXN1210, beginning on Day 29. The safety and tolerability of ALXN1210 in these patients a minimum of 14 days after the first maintenance dose of 900 mg is administered to each of the 2 patients in Cohort 1a is assessed. If no safety concerns are identified, Cohort 2 is enrolled.

Patients in Cohort 2 receive induction doses of 600 mg on Day 1 followed by 900 mg on Day 15. On Day 29, they receive the first of 5 monthly maintenance doses of 1800 mg of ALXN1210. Fourteen days after the fourth dose is administered to the first 2 patients in Cohort 2, the cumulative safety and efficacy data is evaluated for all patients to confirm that all patients in all cohorts can continue dosing at the same dosing levels and frequency.

Following 24 weeks of induction and maintenance treatment, patients enter an extension period of 2 years and continue treatment with ALXN1210 at the same maintenance dose and frequency.

3. Dose Rationale

Based on the observed 32-day half-life for ALXN1210, PD data from in vitro hemolytic assays and measurement of PD markers (free C5 serum concentrations), a drug concentration of ALXN1210 above 100 μg/mL is expected to induce and maintain complete terminal complement inhibition. It is anticipated that the starting dose of 400 mg in Cohort 1a will result in complete terminal complement inhibition for 2 days, and for the entire dosing interval after the second 400 mg dose. The starting dose of 600 mg in Cohorts 1b and 2 will result in serum drug concentrations that provide the minimum efficacious drug concentration for 7 days after the first dose and from the start to the end of the dosing interval after the second dose. Maintenance dosing supports ongoing terminal complement inhibition.

4. Schedule of Assessments

The Schedule of Assessments is set forth in Tables 2 and Table 3.

TABLE 2
Schedule of Assessments: Screening Through End of Maintenance (Day 169)

| Period | | Induction | | | | Maintenance | | | | | | | |
|---|---|---|---|---|---|---|---|---|---|---|---|---|---|
| Study Day | Screening | 1 | 8 | 15 | 22 | 29 | 43 | 57 | 85 | 113 | 141 | 148 | 169¹ |
| Window | −30 to −1 | ±1 | ±1 | ±1 | ±1 | ±1 | ±2 | ±2 | ±2 | ±2 | ±2 | ±2 | ±2 |
| Informed | X | | | | | | | | | | | | |
| consent | | | | | | | | | | | | | |
| Confirmation of | X | | | | | | | | | | | | |
| meningococcal | | | | | | | | | | | | | |
| vaccination | | | | | | | | | | | | | |
| Medical history | X | | | | | | | | | | | | |
| and | | | | | | | | | | | | | |
| demographics | | | | | | | | | | | | | |
| Virus Serology² | X | | | | | | | | | | | | |
| PNH clone³ | X | | | | | | | X | | X | | | X |
| Height, weight, | X | X | | | | | | | | | | | |
| BMI | | | | | | | | | | | | | |
| Pregnancy test⁴ | X | X | | | | X | X | | X | X | X | | X |
| PNH | X | X | X | X | X | X | X | X | X | X | X | | X |
| symptomatology⁵ | | | | | | | | | | | | | |
| Physical | X | | | | | | | | | | | | X |
| examination | | | | | | | | | | | | | |
| Vital signs | X | X | X | X | X | X | X | X | X | X | X | X | X |
| ECG⁶ | X | X | | X | | X | | | | X | X | | X |
| Chemistry | X | X | X | X | X | X | X | X | X | X | X | X | X |
| including | | | | | | | | | | | | | |
| LDH⁷ | | | | | | | | | | | | | |
| Hematology⁸ | X | X | X | X | X | X | X | X | X | X | X | | X |
| Coagulation | X | X | X | X | X | X | X | X | X | X | X | | X |
| Urinalysis and | X | X | | | | | | | X | | | | X |
| urine chemistry, | | | | | | | | | | | | | |
| spot urine | | | | | | | | | | | | | |
| eGFR | | X | | | | | | | X | | | | X |
| calculation⁹ | | | | | | | | | | | | | |
| BNP | | X | | | | X | | | X | | X | | X |
| Cohort 1a¹⁰ | | 400 | 400 | 600 | | 900 | | 900 | 900 | 900 | 900 | | 900 |
| ALXN1210 | | | | | | | | | | | | | |
| (mg) | | | | | | | | | | | | | |
| Cohort 1b¹⁰ | | 600 | | 600 | | 900 | | 900 | 900 | 900 | 900 | | 900 |
| ALXN1210 | | | | | | | | | | | | | |
| (mg) | | | | | | | | | | | | | |
| Cohort 2¹⁰ | | 600 | | 900 | | 1800 | | 1800 | 1800 | 1800 | 1800 | | 1800 |
| ALXN1210 | | | | | | | | | | | | | |
| (mg) | | | | | | | | | | | | | |
| PK sampling¹¹ | | X | X | X | X | X | X | X | X | X | X | X | X |
| Serum | | X | X | X | X | X | X | X | X | X | X | X | X |
| PD panel¹² | | | | | | | | | | | | | |
| Immunogenicity¹³ | | X | | | | X | | X | X | X | X | | X |
| QoL | | X | | | | | X | | | | | | X |
| Assessments¹⁴ | | | | | | | | | | | | | |
| Review safety | | X | X | X | X | X | X | X | X | X | X | X | X |
| card | | | | | | | | | | | | | |

| | |
|---|---|
| Infusion site | ← Monitored continuously → |
| evaluation¹⁵ | |
| Concomitant | ← Monitored continuously → |
| medications | |
| Adverse events | ← Monitored continuously → |

Abbreviations: BMI = body mass index; ECG = electrocardiogram; LDH = lactate dehydrogenase; MCV4 = tetravalent meningococcal conjugate vaccine; PD = pharmacodynamic; PK = pharmacokinetic; PNH = paroxysmal nocturnal hemoglobinuria; QoL = quality of life; BNP = brain natriuretic peptide
Footnotes for Table 2 are provided below:
¹All assessments are performed pre-dose and are part of the Maintenance Period. The dose administered on Day 169 is the first dose in the Extension Period.
²Hepatitis B and C, human immunodeficiency virus (HIV) types 1 and 2.
³Granulocyte and red blood cell (RBC) clone size at screening, and RBC clone size only during the Treatment Period and Extension Period
⁴Female patients only. Serum pregnancy test at screening, end of study, and early termination only. Urine pregnancy tests at all other time points
⁵Investigator assessment of the following events: fatigue, chest pain, abdominal pain, dyspnea, dysphagia, and erectile dysfunction. Symptoms of disease burden is captured through the quality of life (QoL) questionnaires.
⁶Triplicate electrocardiogram (ECG) assessments is conducted at predose and 15 minutes after the end of infusion on Days 1, 15, 29, 113, and 141. The Day 169 ECG assessment is completed prior to dosing.
⁷Clinical safety laboratory measurements is collected predose. Follicle-stimulating hormones and estradiol levels are measured at screening only to confirm postmenopausal status.
⁸Assessment for safety as well as the following parameters as secondary endpoints will be collected predose: free hemoglobin, haptoglobin, reticulocyte count, and D-dimer.
⁹Estimated glomerular filtration rate (eGFR) will be calculated using the Modification of Diet in Renal Disease formula.
¹⁰Patients will be evaluated for clinical safety for at least 4 hours from end of infusion for all doses through Day 169.
¹¹Predose (up to 1 hour before), end of infusion (EOI) (up to 1 hour 30 min after) and 4 hours post-start of infusion (SOI) (±1 hour) on Days 1, 8 (for Cohort 1a only), 15, 29, 57, 85, 113, and 141; anytime on Days 22, 43, and 148; predose (up to 1 hour before) on Day 169
¹²To include serum for storage for exploratory PD assays
¹³Immunogenicity samples will be collected predose.
¹⁴functional Assessment of Chronic Illness Therapy (FACIT), Version 4.0 and European Organisation for Research and Treatment of Cancer, Quality of Life Questionnaire-Core 30 Scale, Version 3.0 (EORTC)
¹⁵Induration or reaction < 1 cm will not be listed as an AE unless it persists for more than 24 hours. Pain at site of infusion will be assessed using a visual assessment scale.

TABLE 3
Schedule of Assessments: Extension Period and Early Termination

| | Extension | | | | |
|---|---|---|---|---|---|
| | | | Every 28 days | | |
| | | | following Day | Every 6 mo | |
| Period | | | 225 until | following Day | Early |
| Study Day | 197 | 225 | Day 897 | 225 until | Termination ¹ |
| Window | ±5 | ±5 | ±5 | Day 897 | ±2 |
| PNH symptomatology ² | X | X | X | | X |
| PNH clone ³ | | | | | X |
| Cohort 1a/b | 900 | 900 | 900 | | |
| ALXN1210 (mg) | | | | | |
| Cohort 2 | 1800 | 1800 | 1800 | | |
| ALXN1210 (mg) | | | | | |
| Vital signs | X | X | | | X |
| Chemistry | X | X | | | X |
| Hematology ⁴ | X | X | | | X |
| Coagulation | X | X | | | X |
| Height, weight, BMI | | | | | X |
| Pregnancy test | X | X | X | | X |
| Physical examination | | | | X | X |
| ECG | | | | X | X |
| Urinalysis and urine | | | | X | X |
| chemistry, spot urine | | | | | |
| Immunogenicity ⁵ | | | X | | X |
| QoL Assessments ^{6, 7} | | | X | | X |
| Review safety card | X | X | X | | X |

| | | |
|---|---|---|
| Infusion site evaluation ⁸ | ← Monitored continuously → | X |
| Concomitant medications | ← Monitored continuously → | X |
| Adverse events | ← Monitored continuously → | X |

Abbreviations: BMI = body mass index; ECG = electrocardiogram; ET = early termination; QoL = quality of life
¹ The early termination visit is only for patients who withdraw or are withdrawn early from the study.
² Investigator assessment of the following events: fatigue, abdominal pain, chest pain, dyspnea, dysphagia, and erectile dysfunction. Symptoms of disease burden will be captured through the QoL questionnaires.
³ PNH RBC clone: Day 253 and Day 337, then every 6 months.
⁴ Assessment for safety as well as the following parameters as secondary endpoints will be collected predose: free hemoglobin, haptoglobin, reticulocyte count, and D-dimer.
⁵ Once on Day 349 and only if there was a positive antidrug antibody result during the induction or maintenance periods.
⁶ Every 3 months starting on Day 253.
⁷ FACIT and EORTC Quality of Life Questionnaire-Core 30 Scale, Version 3.0
⁸ Induration or reaction <1 cm will not be listed as an AE unless it persists for more than 24 hours. Pain at site of infusion will be assessed using a visual assessment scale.

5. Selection and Withdrawal of Patients

All patients are screened for study eligibility after providing written informed consent to participate. Patients who fail any of the eligibility criteria are rescreened once, at the discretion of the Investigator. If the condition that affected eligibility is transient, self-limited, not clinically significant and/or easily treatable, and is resolved at the time of dosing, the patient can enroll as agreed by the Investigator.

Patient inclusion criteria:
1. Male or female ≥18 years of age.
2. PNH diagnosis confirmed by documented high-sensitivity flow cytometry.
3. Mean LDH≥3× upper limit of normal (ULN), based on 2 measurements from separate blood samples collected at least 1 day apart during screening.
4. Willing and able to give written informed consent and comply with the study visit schedule.
5. Documented meningococcal vaccination not more than 3 years prior to dosing.
6. Female patients who consider themselves postmenopausal must provide evidence at screening of menopause based on a combination of amenorrhea for at least 1 year and increased serum follicle-stimulating hormone level (>30 IU/L) on at least 2 occasions (e.g., in the absence of hormone replacement therapy, dietary phytoestrogens), or estradiol concentration <10 pg/mL.
7. Female patients of childbearing potential must use highly effective contraception as defined below, starting at screening and continuing until at least 6 months after the last dose of ALXN1210. Highly effective contraceptive methods are (a) combined (estrogen and progestogen) hormonal contraception associated with inhibition of ovulation (i.e., oral, intravaginal, transdermal), (b) progesterone-only hormonal contraception associated with inhibition of ovulation (i.e., oral, injectable, implantable), (c) intrauterine device, (d) intrauterine hormone-releasing system, and (e) bilateral tubal occlusion.
8. Male patients with a female spouse/partner of childbearing potential or a pregnant or breastfeeding spouse or partner must agree to use barrier contraception (male condom) during the treatment period and for at least 6 months after the last dose of ALXN1210.

Barrier contraception is required even with documented medical assessment of surgical success of a vasectomy. Female spouses or partners of male subjects who are of childbearing potential must use highly effective contraception (simultaneous use of male condom and appropriate barrier methods for the female partner) or acceptable contraception, starting at screening and continuing until at least 6 months after the last dose of ALXN1210. Male subjects must not donate sperm during the screening and treatment periods and for at least 6 months after the last dose of ALXN1210.

Patient exclusion criteria:
1. Treatment with a complement inhibitor at any time.
2. Platelet count <30,000/mm³ (30×10⁹/L) at screening visit.
3. Absolute neutrophil count <500/0 (0.5×10⁹/L) at screening visit.
4. History of bone marrow transplantation.
5. History of N meningitidis infection; history of unexplained, recurrent infection; or infection requiring treatment with systemic antibiotics within the last 90 days prior to dosing,
6. Females planning to become pregnant, or are pregnant or breastfeeding.
7. Positive pregnancy test at screening or Day 1.
8. Patients are excluded if they are taking: (a) erythropoietin or immunosuppressants and are not on a stable dose for at least 26 weeks prior to screening, (b) corticosteroids and are not on a stable dose for at least 4 weeks prior to screening, (c) vitamin K antagonists (Coumadin, warfarin), but did not have a stable international normalized ratio (INR) level for 4 weeks prior to screening, (d) iron supplements or folic acid, but have not been on a stable dose for 4 weeks prior to screening, or (e) low molecular weight heparin, but have not been on a stable dose for 4 weeks prior to screening.
9. Unexplained alanine amino transferase (ALT) or aspartate aminotransferase (AST)>ULN of the testing laboratory at screening.
10. Human immunodeficiency virus (HIV) infection (evidenced by HIV-1 or HIV-2 antibody titer).
11. Acute or chronic hepatitis B virus (HBV) infection (evidenced by the presence of hepatitis B surface antigen [HBsAg] or immunoglobulin M [IgM] antibodies against hepatitis B core antigen [HBcAg]).
12. Acute or chronic hepatitis C virus (HCV) infection (evidenced by antibody titer).
13. Active systemic bacterial, viral, or fungal infection within 14 days prior to dosing.
14. Immunization with a live-attenuated vaccine 1 month prior to dosing, or planned vaccination during the study.
15. Participation in a clinical study within 30 days before initiation of dosing on Day 1, or use of any experimental therapy within 30 days prior to dosing on Day 1, or within 5 half-lives of the product, whichever is greater.
16. Major surgery within 90 days prior to dosing,
17. Presence of fever (body temperature >37.6° C., e.g., associated with a symptomatic viral or bacterial infection) within 2 weeks prior to the first dosing.
18. Patients with a history of malignancy within 5 years of screening with the exception of a nonmelanoma skin cancer or carcinoma in situ of the cervix that has been treated with no evidence of recurrence.
19. Known history of severe allergic or anaphylactic reactions to any drug (including vaccines) or allergen.
20. History of allergy to excipients of ALXN1210 (ie, polysorbate 80).
21. Known allergy to Chinese hamster ovary (CHO) cell proteins.
22. History of any clinically significant cardiac, hepatic, immunologic, pulmonary, or rheumatoid disease that would preclude participation.
23. Inability to comply with study requirements,
24. Other unspecified reasons that make the patient unsuitable for enrollment.

A patient can withdraw from the study at any time at his/her own request, or can be withdrawn at any time at the discretion of the Investigator, for safety, behavioral, or administrative reasons. Patients who discontinue dosing re instructed to return for follow-up visits, unless they withdraw consent and/or are lost to follow-up.

If the patient withdraws consent the Early Termination visit should be as soon as possible. Oral antibiotic therapy is recommended until complement activity is restored.

If the patient is taken off of ALXN1210 and put on rescue with eculizumab then the early termination visit should occur 30 days after the first dose of eculizumab.

If the patient is taken off of ALXN1210 and is willing to return for the remainder of the protocol visits, the patient should do so until they complete Day165 (5.5× half-life) from last dose of ALXN1210.

Patients are permanently discontinued from ALXN1210 treatment if any of the following occur during the study: (a) Serious infusion reaction (such as bronchospasm with wheezing or requiring ventilator support or symptomatic hypotension) or serum sickness-like reactions manifesting 1 to 14 days after drug administration, (b) severe uncontrolled infection, (c) pregnancy or planned pregnancy, or (d) if the Investigator deem it is in the best interest of the patient.

6. Treatment of Patients

Management of Potential Drug Infusion Reactions: some patients treated with IV infusions of mAbs have experienced concurrent infusion-related reactions with signs or symptoms that can be classified as acute allergic reactions/hypersensitivity reactions or cytokine release syndrome. The signs and symptoms include headache, fever, facial flushing, pruritus, myalgia, nausea, chest tightness, dyspnea, vomiting, erythema, abdominal discomfort, diaphoresis, shivers, hypertension, lightheadedness, hypotension, palpitations, and somnolence. Anaphylaxis might occur at any time during an infusion and patients will be monitored closely prior to and through 1 hour following the end of the infusion of ALXN1210. All adverse events which may indicate an infusion-related response will be graded according to criteria from the Common Terminology Criteria for Adverse Events (CTCAE) v4.0.3.

Before infusion is started, the treating physician and other appropriate personnel, medication (adrenaline, inhaled beta agonists, antihistamines, and corticosteroids), and other requirements to treat anaphylaxis must be readily available.

The infusion is stopped immediately if ≥Grade 2 allergic/hypersensitivity reactions (including drug fever) or ≥Grade 3 cytokine release syndrome/acute infusion reaction occurs. Patients experiencing a reaction during the administration of study drug should be treated according to institutional guidelines.

For a Grade 1 or Grade 2 infusion-related reaction, the infusion is stopped and medication with antihistamine (e.g., with diphenhydramine, 25 to 50 mg orally or equivalent) and acetaminophen (650 mg orally or equivalent) may be considered. If the signs and symptoms have resolved with the above medications, the infusion is restarted. If the infusion is slowed, the total infusion time should not exceed 5 hours, including any interruptions for safety or technical reasons. The study drug is stopped if the infusion reaction recurs. Patients experiencing an infusion reaction are observed in the clinic until resolution of the reaction.

If an event of anaphylaxis occurs, according to the criteria in Table 4 then subcutaneous epinephrine ( 1/1000, 0.3 to 0.5 mL or equivalent) is considered. In the case of bronchospasm, inhaled beta agonist is considered. Patients administered antihistamine for the study drug or prevention of infusion reactions are given appropriate warnings about drowsiness and impairment of driving ability prior to discharge.

Patients who experience a severe reaction during administration of study drug resulting in discontinuation of study drug undergo all scheduled safety, PK, and PD evaluations required by the protocol.

TABLE 4
Clinical Criteria for Diagnosing Anaphylaxis

Anaphylaxis is highly likely when any one of the following 3 criteria are fulfilled:

| | |
|---|---|
| 25. | Acute onset of an illness (minutes to several hours) with involvement of the skin, |
| | mucosal tissue, or both (e.g., generalized hives, pruritus or flushing, swollen lips- |
| | tongue-uvula) |

AND AT LEAST ONE OF THE FOLLOWING:

| | |
|---|---|
| a. | Respiratory compromise (e.g., dyspnea, wheeze-bronchospasm, stridor, reduced PEF, |
| | hypoxemia) |
| b. | Reduced BP or associated symptoms of end-organ dysfunction (e.g., hypotonia |
| | [collapse], syncope, incontinence) |
| 26. | Two or more of the following that occur rapidly after exposure to a likely allergen for that |
| | patient (minutes to several hours): |
| a. | Involvement of the skin-mucosal tissue (e.g., generalized hives, itch-flush, swollen |
| | lips-tongue-uvula) |
| b. | Respiratory compromise (e.g., dyspnea, wheeze-bronchospasm, stridor, reduced PEF, |
| | hypoxemia) |
| c. | Reduced BP or associated symptoms (e.g., hypotonia [collapse], syncope, |
| | incontinence) |
| d. | Persistent gastrointestinal symptoms (e.g., crampy abdominal pain, vomiting) |
| 27. | Reduced BP after exposure to known allergen for that patient (minutes to several hours): |
| a. | Systolic BP of less than 90 mm Hg or greater than 30% decrease from that person's |
| | baseline |

Anaphylaxis is highly likely when any one of the following 3 criteria are fulfilled:
Source: Adapted from Sampson HA, et al., Second symposium on the definition and
management of anaphylaxis: summary report: Second National Institute of Allergy and
Infectious Disease/Food Allergy and Anaphylaxis Network symposium. J Allergy Clin
Immunol. 2006; 117(2): 391-397.
PEF, Peak expiratory flow; BP, blood pressure.

Infection Risk: Due to its mechanism of action, the use of ALXN1210 increases the patient's susceptibility to meningococcal infection (N meningitidis). Patients might be at risk of disease by uncommon serogroups (such as X), although meningococcal disease due to any serogroup may occur. To reduce the risk of infection, all patients are vaccinated prior to receiving ALXN1210. Patients who are treated with ALXN1210 less than 2 weeks after receiving a meningococcal vaccine receive treatment with appropriate prophylactic antibiotics until 2 weeks after vaccination. Vaccines against serotypes A, C, Y, W 135, and B, where available, are recommended to prevent common pathogenic meningococcal serotypes. Patients must be vaccinated or revaccinated according to current national vaccination guidelines or local practice for vaccination use with complement inhibitors (e.g., eculizumab).

Vaccination may not be sufficient to prevent meningococcal infection. Consideration should be given per official guidance and local practice on the appropriate use of antibacterial agents. All patients are monitored for early signs of meningococcal infection, evaluated immediately if infection is suspected, and treated with appropriate antibiotics, if necessary.

To increase risk awareness and promote quick disclosure of any potential signs or symptoms of infection experienced by the patients during the course of the study, patients are provided a safety card to carry with them at all times.

Prior and Concomitant Medications and Procedures: Prior medications (including vitamins and herbal preparations), including those discussed in the exclusion criteria and/or procedures (any therapeutic intervention, such as surgery/biopsy or physical therapy) that the patient takes or undergoes within 28 days prior to signing the informed consent form (ICF) until the first dose of ALXN1210 are recorded on the patient's electronic case report form (eCRF). All medication used during screening and the Treatment and Extension periods are recorded in the patient's source/chart and electronic case report form. This record includes all prescription drugs, herbal products, vitamins, minerals, over-the-counter medications, and current medications for PNH. Any changes in concomitant medications are recorded in the patient's source/chart and electronic case report form. Any concomitant medication deemed necessary for the patient's standard of care treatment during the study, or for the treatment of any adverse event, along with those the allowed medications described is given at the discretion of the Investigator.

The following concomitant medications are allowed if the following apply, and dose adjustments are not expected during the treatment period:
Patients are taking erythropoietin on a stable dose for at least 26 weeks prior to screening.
Patients are taking immunosuppressants on a stable dose for at least 26 weeks prior to screening.
Patients are taking corticosteroids on a stable dose for at least 4 weeks prior to screening.
Patients are allowed to take vitamin K antagonists (Coumadin, warfarin) but must have had a stable international normalized ratio (INR) level for 4 weeks prior to screening.
Patients are taking iron supplements or folic acid on a stable dose for at least 4 weeks prior to screening.
Patients are allowed to take low molecular weight heparin on a stable dose for at least 4 weeks prior to screening.
   Adjustments in the frequency or level of dosing in any of the above medications are made if it is in the best interest of the patient.

Treatment Compliance: Patients are administered ALXN1210 in a controlled setting under the Investigator's supervision, thereby ensuring compliance with ALXN1210 administration. Study coordinators at the investigative site will ensure that all patients are adequately informed on the specific ALXN1210 dosing regimen required for compliance with the study protocol.

Randomization and Blinding. This is an open-label study. The first 2 eligible patients who meet the inclusion/exclusion criteria are assigned on Day −1 to Cohort 1a. If the first two 400 mg doses are safe and tolerable in these first 2 patients, the remaining patients (n=4) are enrolled into Cohort 1b. Fourteen days after the first maintenance dose of 900 mg is administered to the second patient in Cohort 1a, the DMC recommends enrollment of Cohort 2 if no safety risks are identified. Once the first 2 patients are enrolled into Cohort 2, and if Cohort 1b is still not fully enrolled, the enrollment of Cohorts 1b and 2 is balanced in a 1:1 ratio.

7. Study Drug Materials and Management

Each vial of study drug contains 150 mg of ALXN1210 in 10 mM sodium phosphate, 150 mM sodium chloride, 0.02% polysorbate 80, and Water for Injection. ALXN1210 is formulated at pH 7.0 and is presented as a sterile, preservative-free, 10 mg/mL solution for IV administration, supplied in 20-mL single-use vials. ALXN1210 is suitable for human use and manufactured under current Good Manufacturing Practices (GMP).

TABLE 5
Investigational Product

| | Investigational Product |
|---|---|
| Product Name: | ALXN1210 |
| Dosage Form: | Sterile solution for IV administration |
| Unit Dose | 150 mg/vial |
| Route of Administration | Intravenous |
| Manufacturer | Alexion Pharmaceuticals, Inc. |

ALXN1210 is supplied in a one-vial-per-kit configuration. Each vial and carton is labeled according to specific country or region regulatory requirements. ALXN1210 vials are stored in refrigerated conditions at 2° C. to 8° C. (36° F. to 46° F.) and protected from light. ALXN1210 vials are not to be frozen or shaken. Preparation of ALXN1210 doses is performed in accordance with site-specific local standards by qualified and study-trained pharmacy personnel. Handling and preparation of materials used to prepare and administer study drug is carried out using aseptic techniques for sterile products.

Pharmacy personnel prepare doses in accordance with the dose assignment. ALXN1210 is diluted in 0.9% sodium chloride injection (country-specific pharmacopeia) and administered by IV infusion at a fixed rate of 686 mg/hour for doses up to 900 mg, and 880 mg/hour for the 1800 mg dose. For each patient, doses are prepared as required for each dose cohort, as indicated in Table 6.

TABLE 6
Dosing Reference Chart for ALXN1210 Dose Preparation

| | Infusion | Infusion | Infusion | Infusion Duration | |
|---|---|---|---|---|---|
| Dose | Volume | Rate | Rate | in minutes | |
| (mg) | (mL) | (mL/hr) | (mg/hr) | (hr) | |
| 400 | 80 | 137 | 686 | 35.0 | (0.58) |
| 600 | 120 | 137 | 686 | 52.5 | (0.88) |
| 900 | 180 | 137 | 686 | 78.8 | (1.31) |
| 1800 | 360 | 176 | 880 | 122.7 | (2.04) |

For in-use shelf life ALXN1210 is diluted with 0.9% sodium chloride injection (country-specific pharmacopeia) before administration (dosing solution). The dosing solution is stable for 6 hours at room temperature 15° C. to 25° C. (59° F. to 77° F.) and for 24 hours at 2° C. to 8° C. (36° F. to 46° F.). The expiration date and time of the dosing solution is calculated from the time dose preparation is complete. The dose is administered within the expiration date and time.

All doses of ALXN1210 are administered by IV infusion, using a programmable IV infusion pump and IV sets with in-line filters, at a fixed rate of 686 mg/hour for the doses up to 900 mg, and 880 mg/hour for the 1800-mg dose. Total infusion time including any interruptions for safety or technical reasons, will not exceed 5 hours.

During the induction and maintenance periods (through Day 141), patients remain seated or semi-reclined for the time of drug administration and until at least 2 hours after end of infusion (EOI), and remain in the clinic for an additional 2 hours for safety observations. Failure of patients to comply with this requirement does not constitute a deviation from the protocol if it is medically necessary, procedurally required, or to go to the bathroom. When appropriate, patients will be accompanied by a staff member during ambulation.

Time of dosing (t=0) is defined as ALXN1210 start of infusion (SOI). All procedures are performed in relation to SOI or EOI as described in the Schedules of Assessments.

The study site must maintain accurate records demonstrating dates and amount of study drug received, to whom dispensed (patient-by-patient accounting), and accounts of any study drug accidentally or deliberately destroyed. Accountability logs are provided to assist the pharmacist in maintaining current and accurate inventory records covering receipt, dispensing, and disposition of the study drug. The study monitor examines the inventory during the study. Accountability records are readily available and may be subject to regulatory authorities, the local regulatory agency, or an independent auditor's inspection at any time.

Unless otherwise notified, empty vials and vials with residual materials are kept for inspection and accountability by the study monitor prior to their destruction or handled per local site pharmacy standard operating procedures for clinical study drugs. At the end of the study, a final reconciliation is made between the amount of study drug supplied, dispensed, and subsequently destroyed or returned. A written explanation is provided for any discrepancies.

8. Pharmacokinetic Assessments

Blood Sample Collection: the total volume of blood collected per patient for clinical laboratory, PK, PD, and immunogenicity assessments does not exceed 300 mL in any 16-week period.

After ALXN1210 administration, blood samples for determination of serum ALXN1210 concentrations are collected at the time points indicated in the Schedule of Assessments, with the actual blood sampling dates and times being recorded and used in PK calculations. The timing of PK sample collection is altered based on initial PK results to ensure appropriate PK monitoring. The number of PK sampling time points for any given patient does not exceed the currently planned number of time points.

TABLE 7
Collection Time Points for Pharmacokinetic and Pharmacodynamic Analyses

| Cohort | Dose | Collection Time Points (windows) |
|---|---|---|
| 1a | Dose 1 (400 mg) on Day 1 | Predose (up to 1 hour before) |
| | | EOI (up to 0.5 hour after) |
| | | 4 hours post-SOI (±1 hour) |
| | Dose 2 (400 mg) on Day 8 | Predose of each dose (up to 1 hour before) |
| | Dose 3 (600 mg) on Day 15 | EOI of each dose (up to 0.5 hour after) |
| | Doses 4 to 7 (900 mg) | 4 hours post-SOI of each dose (±1 hour) |
| | on Days 29, 57, 85, 113 | Days 22 (±1 day) and 43 (±2 days) |
| | Doses 8 to 9 (900 mg) | Predose on Day 141 (up to 1 hour before) |
| | on Days 141 and 169 | EOI on Day 141 (up to 0.5 hour after) |
| | | 4 hours post-SOI on Day 141 (±1 hour) |
| | | 7 days after dosing (Day 148 ±2 days) |
| | | Predose on Day 169 (up to 1 hour before) |
| | | When a patient discontinues the study |
| 1b | Dose 1 (600 mg) on Day 1 | Predose (up to 1 hour before) |
| | | EOI (up to 0.5 hour after) |
| | | 4 hours post-SOI (±1 hour) |
| | | 7 days post-dose (Day 8 ±1 day) |
| | Dose 2 (600 mg) on Day 15 | Predose of each dose (up to 1 hour before) |
| | Doses 3 to 6 (900 mg) | EOI of each dose (up to 0.5 hour after) |
| | on Days 29, 57, 85, 113 | 4 hours post-SOI of each dose (±1 hour) |
| | | Days 22 (±1 day) and 43 (±2 days) |
| | Doses 7 to 8 (900 mg) on | Predose on Day 141 (up to 1 hour before) |
| | Days 141 and 169 | EOI on Day 141 (up to 0.5 hour after) |
| | | 4 hours post-SOI on Day 141 (±1 hour) |
| | | 7 days after dosing (Day 148 ±2 days) |
| | | Predose on Day 169 (up to 1 hour before) |
| | | When a patient discontinues the study |
| 2 | Dose 1 (600 mg) on Day 1 | Predose (up to 1 hour before) |
| | | EOI (up to 0.5 hour after) |
| | | 4 hours post-SOI (±1 hour) |
| | | 7 days post-dose (Day 8 ±1 day) |
| | Dose 2 (900 mg) on Day 15 | Predose of each dose (up to 1 hour before) |
| | Dose 3 (1800 mg) on Day 29 | EOI of each dose (up to 0.5 hour after) |
| | Doses 4 to 6 (1800 mg) | 4 hours post-SOI of each dose (±1 hour) |
| | on Days 57, 85, 113 | Days 22 (±1 day) and 43 (±2 days) |
| | Doses 7 to 8 (1800 mg) on | Predose on Day 141 (up to 1 hour before) |
| | Days 141 and 169 | EOI on Day 141 (up to 0.5 hour after) |
| | | 4 hours post-SOI on Day 141 (±1 hour) |
| | | 7 days after dosing (Day 148 ±2 days) |
| | | Predose on Day 169 (up to 1 hour before) |
| | | When a patient discontinues the study |

9. Immunogenicity Assessments

Blood samples for the assessment of antidrug antibody (ADA) to ALXN1210 are collected at selected time points coinciding with laboratory assessments: pre-dose on Day 1, Day 29, Day 57, Day 85, Day 113, Day 141, Day 169, and at early termination. If an ADA assessment is positive during the study, another sample is collected at 180 days after the last dose of the maintenance period (Day 349 or as appropriate for patient that undergoes early termination). The immunogenicity assay evaluates ADA to ALXN1210.

10. Assessment of Efficacy

Blood samples for chemistry are used to measure Lactate Dehydrogenase (LDH) levels at the time points described in the Schedule of Assessments.

Biomarkers of PNH: A serum PD panel is collected for analyses of C5 levels (total and free), cRBC hemolysis, and quantitative measures of C5 activation at the time points indicated in Table 7. In addition, serum samples are stored for potential additional analyses.

Hemolysis-related hematological parameters are assessed by measurements of free hemoglobin, haptoglobin, reticulocyte count, PNH RBC clone size (%), and D-dimer. Blood samples for analysis of these parameters are taken at the time points described in the Schedule of Assessments.

Investigator assessment of clinical symptoms related to PNH is made at the time points described in the Schedule of Assessments. The Investigator assesses patients for the following events: fatigue, abdominal pain, chest pain, dyspnea, dysphasia, and erectile dysfunction. Symptoms of disease burden are captured through the quality of life (QoL) questionnaire.

Markers of PNH symptoms and comorbidities (i.e., chronic kidney disease by urinary spot albumin:creatinine ratio and (estimated glomerular filtration rate) eGFR, and (brain natriuretic peptide) BNP for pulmonary hypertension) is evaluated in the study as exploratory efficacy endpoints. Evaluation for changes in kidney function is based on investigator assessment and laboratory results of serum and urinary creatinine and eGFR. Results of eGFR are calculated using the Modification of Diet in Renal Disease formula at the same time points when blood is drawn for chemistry assessments as described in the Schedule of Assessments.

Quality of Life: The FACIT-F scale (Version 4.0) is a collection of quality of life (QoL) questionnaires targeted to the management of fatigue symptoms due to a chronic illness. The European Organization for Research and Treatment of Cancer, Quality of Life Questionnaire-Core 30 Scale, Version 3.0 (EORTC scale) is a questionnaire developed to assess the QoL of cancer patients. Both scales are administered at the time points described in the Schedule of Assessments.

Major adverse vascular events (MAVE) are assessed as part of the planned evaluation for adverse events. The definition of a MAVE is provided below.
The description of event, location, method of diagnosis (magnetic resonance imaging [MRI], ultrasound, angiogram, or other), date of diagnosis and date resolved (or ongoing) will be collected on the electronic case report form as part of the patient's medical history and during the study.
   A MAVE can be only of the following events:
Thrombophlebitis/deep vein thrombosis
Pulmonary embolus
Myocardial infarction
Transient ischemic attack
Unstable angina
Renal vein thrombosis/renal artery thrombosis/glomerular thrombosis, renal infarction
Acute peripheral vascular occlusion
Mesenteric/visceral vein/arterial thrombosis or infarction
Hepatic/portal vein thrombosis
Cerebral arterial occlusion/cerebrovascular accident
Cerebral venous occlusion
Renal arterial thrombosis
Multi-infarct dementia

11. Assessment of Efficacy

Patients meet with the Investigator or designee to discuss the potential safety risks of ALXN1210 and to allow for the Investigator to address any of the patient's safety concerns at the time points shown in the Schedule of Assessments.

Collection of adverse events (including serious adverse events (serious adverse events) and MAVEs) are monitored from the time informed consent is obtained until study completion. Investigators follow any adverse events through to their conclusion. In the event of patient discontinuation from the study, adverse event monitoring continues through the last study visit, if possible. Clinical and laboratory assessments are performed to assess ALXN1210 safety. Timing of the assessments is described in the Schedule of Assessments. Any abnormal results are followed until resolution or stabilization.

A review of demographic parameters, including age, gender, race, and ethnicity is performed, as described in the Schedule of Assessments. A complete medical history is taken and documented.

Vital signs are taken after the patient has been resting in the supine position for at least 5 minutes, and include temperature (° C.; oral), respiratory rate, supine blood pressure, and pulse. The timing of vital sign assessments is described in the Schedule of Assessments. Out-of-range blood pressure or pulse measurements are repeated at the Investigator's discretion. Any confirmed, clinically significant vital sign measurements are recorded as adverse events.

Weight, height, and body mass index (BMI) are recorded, as described in the Schedule of Assessments. A physical examination assessing general appearance, skin, head/eyes/ears/nose/throat, neck, lymph nodes, chest, heart, abdominal cavity, limbs, central nervous system and musculoskeletal is performed.

A triplicate 12-lead ECG is obtained after the patient has been resting for at least 5 minutes at predose, and 15 minutes after end of infuson for the time points described in the Schedule of Assessments. Heart rate, PR, QRS, RR, and QT are measured and corrected intervals (Fridericia formula) are calculated.

Blood samples for analysis of hematology, chemistry, coagulation, urinalysis/urine chemistry, virus serology and other parameters are collected as described in the Schedule of Assessments. Abnormal results are followed, as appropriate.

Blood samples are analyzed for chemistry parameters. Considering that indirect bilirubin is calculated from total and direct bilirubin values, indirect bilirubin results are not be available if direct bilirubin is below the limit of quantification. Serum FSH levels and estradiol concentrations are measured at screening for postmenopausal female patients to confirm their postmenopausal status. Timing of chemistry assessments is described in the Schedule of Assessments.

Blood samples are analyzed for prothrombin time, INR, and partial thromboplastin time. Timing of coagulation assessments is described in the Schedule of Assessments.

Urinalysis includes specific gravity, pH, glucose, protein, blood, nitrates, and ketones. A microscopic examination of urine samples is performed only on abnormal findings. Urine samples are also analyzed to measure proteins and creatinine to calculate the urine protein:creatinine ratio. Timing of urinalysis and urine chemistry assessments is described in the Schedule of Assessments.

Blood samples collected at screening are analyzed for HIV-1, HIV-2, HBsAg, IgM, anti-HBcAg, and HCV antibody titers.

A serum pregnancy test (beta human chorionic gonadotrophin) is performed in all female patients at screening, end of study, and early termination only. A urine pregnancy test is performed at all other time points. The timing of pregnancy testing is described in Schedule of Assessments.

Evaluation for administration site reaction is made at the time point described in Schedule of Assessments. Administration site reactions are recorded as an adverse event using the appropriate coding terms.

An induration or reaction of <1 cm is not be listed as an adverse event unless it persists for more than 24 hours. Pain at site of infusion is assessed using a visual assessment scale with assessment recorded as centimeters.

12. Adverse Event Management

The Investigator is responsible for detecting, assessing, documenting and reporting all adverse events. All adverse events are recorded from the signing of informed consent until study completion. There is no time limit for severe adverse events that are considered causally related.

All observed or volunteered adverse events, regardless of causal relationship, must be reported and recorded in the elecetronic case report form. Adverse events reported by the patient and/or parent or legal guardian, and/or identified in response to an open-ended question from study personnel, or revealed by observation, physical examination, or other study procedures are collected and recorded.

An adverse event is defined as any unfavorable and unintended sign (e.g., including an abnormal laboratory finding), symptom, or disease temporally associated with the use of a medicinal product or procedure, whether or not considered related to the medicinal product or procedure, which occurs during the course of the clinical study. Exacerbations of a chronic or intermittent pre-existing condition, including either an increase in frequency and/or intensity of the condition, are all to be considered adverse events.

Abnormal test findings may be considered adverse events. If an abnormal laboratory value is identified, Investigators are encouraged to report a diagnosis, or a sign or symptom, rather than an isolated abnormal test value. An abnormal test finding should be documented as an adverse event if any of the following conditions are met:
Is associated with a sign or symptom
Requires additional diagnostic testing (repeat tests are not considered additional testing)
Requires a medical or surgical intervention
Leads to a change in study dosing outside of the protocol-defined dosing or leads to discontinuation from the study
Requires significant additional treatment
Does not meet any of the conditions above; however, the Investigator or Sponsor considers the result clinically significant or meeting the definition of an adverse event.

This definition also includes the signs or symptoms resulting from:
Drug overdose
Drug withdrawal
Drug misuse
Drug interactions
Extravasation
Exposure during pregnancy
Exposure via breastfeeding
Medication error
Occupational exposure

An adverse event does not necessarily include the following:
Medical or surgical procedures (e.g., surgery, endoscopies, tooth extraction, transfusion); the condition that leads to the procedure is the AE (e.g., laparoscopic cholecystectomy is the procedure or treatment for an serious adverse event of necrotic gall bladder)
Pre-existing diseases or conditions present or detected prior to the screening evaluation that do not worsen
Situations where an untoward medical occurrence has not occurred (e.g., hospitalization for elective surgery if planned prior to the start of the study, social and/or convenience admissions)

Any adverse event that fulfills any 1 of the criteria listed below is recorded as a serious adverse event. A serious adverse event is described as any untoward medical occurrence that, at any dose:
Results in death
Is life threatening
Requires hospitalization or prolongation of hospitalization^{b}. Hospitalization does not necessarily include the following:
   Rehabilitation/hospice/nursing facility
   Emergency Room visit less than 24 hours
   Elective or preplanned admission/surgery/day surgery
   Protocol-specified admission
   Admission for a pre-existing condition not associated with either a new adverse event or with worsening of a pre-existing adverse event
Results in persistent or significant disability/incapacity
Is a congenital anomaly/birth defect
Is an important medical event

The term “life threatening” in the definition of “serious” refers to an event in which the patient was at risk of death at the time of the event. It does not refer to an event which hypothetically might have caused death if it were more severe.

Hospitalization requires inpatient admission or prolongation of an existing hospitalization. The adverse events that are associated with hospitalization or prolongation of hospitalization are considered serious adverse events.

Important medical event: Medical and scientific judgment is exercised in deciding whether expedited reporting is appropriate in other situations, such as important medical events that may not be immediately life threatening, or result in death or hospitalization, but may jeopardize the patient or may require intervention to prevent 1 of the other outcomes listed in the definition above. These are also usually be considered serious. Examples of such events are intensive treatment in an emergency room or at home for allergic bronchospasm; blood dyscrasias or convulsions that do not result in hospitalization; or development of drug dependency or drug abuse.

Severity and seriousness are differentiated. Severity describes the intensity of an adverse event, while the term seriousness refers to an adverse event that has met the criteria for a serious adverse event, as described above.

All adverse events re graded according to criteria from CTCAE v4.03, published Jun. 14, 2010.
Grade 1: Mild (awareness of sign or symptom, but easily tolerated)
Grade 2: Moderate (discomfort sufficient to cause interference with normal activities)
Grade 3: Severe (incapacitating, with inability to perform normal activities)
Grade 4: Life threatening
Grade 5: Fatal

Changes in the severity of an adverse event are documented to allow an assessment of the adverse event duration at each level of intensity to be evaluated. Adverse events characterized as intermittent require documentation of onset and duration of each episode, if the severity of the intermittent event changes.

An Investigator causality assessment is provided for all adverse events (both nonserious and serious). This assessment is recorded in the electronic case report form and on any additional forms, as appropriate. The definitions for the causality assessments are as follows:
Not related (unrelated): This relationship suggests that there is no association between the investigational product and the reported event.
Unlikely related: This relationship suggests that the clinical picture is highly consistent with a cause other than the investigational product, but attribution cannot be made with absolute certainty, and a relationship between the investigational product and adverse event cannot be excluded with complete confidence.
Possibly related: This relationship suggests that treatment with the investigational product may have caused or contributed to the adverse event, i.e., the event follows a reasonable temporal sequence from the time of drug administration, and/or follows a known response pattern to the investigational product, but could also have been produced by other factors.
Probably related: This relationship suggests that a reasonable temporal sequence of the event with the investigational product administration exists, as well as the likely association of the event with the investigational product. This will be based upon the known pharmacological action of the investigational product, known or previously reported adverse reactions to the investigational product or class of drugs, or judgment based on the Investigator's clinical experience.
Definitely related: Temporal relationship to the investigational product. Other conditions (concurrent illness, concurrent medication reaction, or progression/expression of disease state) do not appear to explain event, corresponds with the known pharmaceutical profile, improvement on discontinuation, reappearance on rechallenge.

For all adverse events, regardless of causal relationships, the Investigator must follow up regarding the outcome of the event until the event or sequelae either resolve or stabilize. Adverse event outcomes must be recorded in the electronic case report form and on any additional forms, as appropriate.

If a patient experiences a serious adverse event with an outcome of death, the following procedures are to be performed:
The serious adverse event resulting in death has an outcome documented as death/fatal, with an end date being the date of death.
If the patient had additional adverse event/serious adverse events that were ongoing at the time of death, these events are documented as ongoing with no end date.
Only 1 event has an outcome of death/fatal, unless an autopsy report or Investigator states otherwise.

All observed or volunteered adverse events, regardless of dose cohort or causal relationship, are reported. For all adverse events, the Investigator must do the following:

1. Determine the adverse event outcome

2. Determine if the event meets criteria for a serious adverse event

3. Assess adverse event severity

4. Determine adverse event causality

Adverse events are documented in clear, unambiguous medical terms. Study personnel are advised not to use abbreviations or acronyms.

For each adverse event, only the diagnosis is recorded on the electronic case report. Characteristic signs and symptoms of the diagnosis are not reported as additional adverse events. If a diagnosis is not available, each sign and symptom is recorded as an adverse event. When a diagnosis becomes available, the source document and the electronic case report art updated with the relevant diagnosis only.

For medical or surgical procedures (e.g., surgery, endoscopies, tooth extraction, transfusion), the condition/diagnosis that leads to the procedure is recorded as the adverse event (e.g., laparoscopic cholecystectomy is the procedure or treatment for an serious adverse event of necrotic gall bladder).

All adverse events that later increase in frequency and or severity (medical and scientific judgment should be exercised by the Investigator) are considered new adverse events, and re recorded on a new line in the electronic case report form.

Withdrawal due to an adverse event or serious adverse event is clearly differentiated from withdrawal due to other reasons.

All adverse events are assessed by the Investigator to determine if they meet criteria for a serious adverse event. All serious adverse events are reported immediately, or within 24 hours of the Investigator and/or study site staff becoming aware of the event, regardless of the presumed relationship to the study drug.

Pregnancy data is collected for all patients. Pregnancy in itself is not regarded as an adverse event, unless there is a suspicion that investigational product may have interfered with the effectiveness of a contraceptive medication. However, complications of pregnancy and abnormal outcomes of pregnancy are adverse events, and many may meet criteria for a serious adverse event. Complications of pregnancy and abnormal outcomes of pregnancy, such as ectopic pregnancy, spontaneous abortion, intrauterine fetal demise, neonatal death, or congenital anomaly, meet the criteria of a serious adverse aevent and therefore are reported as such. Elective abortions without complications should not be handled as an adverse event.

13. Statistics

All data collected is documented using summary tables, figures, and data listings. For categorical variables, frequencies and percentages are presented for each cohort, and for the combined cohorts. For continuous variables, descriptive statistics (n, mean, median, SD, minimum, maximum) are presented for each cohort, and for the combined cohorts.

Descriptive statistics for PK parameters include the number of observations, mean, SD, coefficient of variance (CV), median, minimum, maximum, geometric mean, and geometric % CV.

A clinical study report (CSR) is produced after the end of the maintenance period and includes safety, efficacy, PK, and PD analyses. A final CSR is produced at study completion and includes data on all patients in the study at the end of the extension period.

The Safety Set consists of all patients who received at least 1 dose of ALXN1210. This population is used for the safety analysis.

The PK population consists of all patients who have sufficient serum concentration data to enable the calculation of PK parameters.

The PD analysis population consists of all patients who have both a predose and postdose PD sample collected.

The immunogenicity analysis population consists of all patients who have both a predose and postdose ADA sample collected.

The Full Analysis Set (FAS) consists of all patients in the Safety Set with a baseline and 1 post-treatment LDH measurement.

A sample size of 12 patients from the combined arms provides an approximately 80% power to detect a mean paired difference in LDH from baseline of −40%, with an estimated SD of 45%. This was based on a 2-sided, paired t-test, with a 5% type 1 error rate.

All patients are included in the summaries of disposition, which summarizes the number of patients randomized in the study, the frequency and percentage of patients who completed or discontinued from the study, along with reason for discontinuation, by cohort. Demographics and baseline characteristics are summarized for all patients by each cohort and overall.

Safety analyses are performed on the safety population, and are reported by cohort and overall. Safety analyses include all adverse events, electrocardiograms, clinical laboratory data, physical examinations, and vital sign measurements, and are presented using descriptive statistics. No inferential statistical analyses are planned on safety parameters.

The incidence of treatment-emergent adverse events (TEAEs) and serious adverse events is summarized by system organ class and preferred term for each cohort and overall, by severity, and by relationship to ALXN1210. Adverse events re categorized by cohort at the date of onset, and are coded using the Medical Dictionary for Regulatory Activities (MedDRA), Version 18.0 or higher. Serious adverse events and adverse events resulting in withdrawal from the study are listed. Patients having multiple adverse events within a category (e.g., overall, SOC, preferred term) re counted once in that category. For severity tables, a patient's most severe event within a category is counted.

Changes from baseline in vital signs and laboratory assessments (chemistry, complete blood count [CBC] with differential, and urinalysis) are summarized by cohort. Shift tables of clinical laboratory tests (Low, Normal, High) by cohort are produced. Graphical displays are presented, as appropriate.

All concomitant medications are coded using the World Health Organization (WHO) Drug Dictionary, and the frequency and percentage of concomitant medications are summarized.

Absolute LDH levels, and the change and percent change from baseline to Day 169 is summarized at all study visits. Baseline is defined as the average of all available assessments on or prior to first ALXN1210 infusion. A mixed model for repeated measures with the fixed, categorical effect of visit and fixed, continuous effect of baseline LDH levels as covariates is fit to test whether changes and percent changes differ from zero at each time point. An unstructured covariance analysis is used to model the within-patient errors. If this analysis fails to converge, the following structures re tested, and the final covariance structure is determined by Akaike's information criterion: first order autoregressive, compound symmetry, and Toeplitz method. The Kenward-Roger approximation is used to estimate denominator degrees of freedom. As a sensitivity analysis, changes and percent changes from baseline are analyzed using the Wilcoxon signed-rank test. Graphical displays are presented, as appropriate. The percentage of patients with clinical symptoms is summarized for all study visits.

Changes in hematologic measures are similarly analyzed using a mixed effect model repeat measurement (MMRM), and Wilcoxon signed-rank test.

Scoring guidelines for the FACIT-F and EORTC scales are used to calculate QoL scores. Changes from baseline in FACIT-F and EORTC scale scores are summarized descriptively for all study visits, and analyzed using MMRM and Wilcoxon signed rank test.

Transfusion rates and the incidence rate of MAVEs are summarized.

Individual serum concentration data for ALXN1210-treated patients, with actual sampling dates and times, is used to derive the PK parameters by noncompartmental analyses, using Phoenix WinNonlin 6.3 or higher. Mean serum ALXN1210 concentrations versus nominal time, and individual serum ALXN1210 concentrations versus actual time is graphically presented.

The following PK parameters are estimated: Cₘₐₓ, tₘₐₓ, AUCₜ, AUC₀₋ₜ, λ_{z}, t_{1/2}, CL, Vss, Cₘᵢₙ. Assessment of steady state and accumulation at steady state also are evaluated. Additional PK analyses, such as assessment of PK linearity, can be conducted.

Descriptive statistics (mean, SD, CV, median, minimum, maximum, geometric mean, and geometric % CV) of the serum concentration and PK parameter summaries are provided, as appropriate.

The PD effects of ALXN1210 administered IV are evaluated by assessing changes and percent changes in serum total and/or free C5 concentrations, cRBC hemolysis, and other measures of C5 activation over time. Assessments of PK-PD relationships are explored using data from this study or in combination of data from other studies.

Immunogenicity, as measured by ADA, is summarized in tabular form by treatment dose.

### Example 2: Preliminary Results from Open-Label, Intrapatient, Dose-Escalation Study in PNH Patients

The following is a summary of Lactate dehydrogenase (LDH) data from an ongoing open-label, multiple-dose, multi-center intrapatient dose-escalation study according to the protocol described above in Example 1. LDH levels were assessed in patients according to the assay described below.

1. Materials and Methods—LDH Assay

LDH levels were assessed using an LDH assay manufactured by Roche Diagnostics Corporation (Indianapolis, Ind.), which includes the Reagents: R1) Buffer/lactate and R2) Coenzyme. The assay was performed on Roche Modular and Cobas Analyzers, according to the manufacturer's protocol, which is summarized below.

L-lactate+NAD+-LD→pyruvate+NADH+ H+ NAD and lactate are converted in equimolar amounts at the same time. The rate at which NADH is formed is determined by an increase in absorbance and is directly proportional to enzyme activity and can be measured photometrically.

Serum samples were utilized. Stability of the specimen was 5 days ambient and 5 days at refrigerated temperature. Samples were not stable at frozen temperature. Testing was performed daily and a minimum volume of 350 μL was required for analysis.

A two point calibration was performed daily. Roche C.f.a.s. Calibrator and 0.9% saline was used for calibration. The method was standardized by manual measurement against the original IFCC formulation.

The expected intra-assay precision was 0.3-1.0% CV. The CLS acceptable limit was less than 2.0% CV. The following demonstrates intra-assay precision determined by replicate assays of control material in a single run.

TABLE 8
Intra-Assay Precision

| | Level I | Level II |
|---|---|---|
| Mean (U/L) | 109.52 | 394.23 |
| SD | 0.87 | 3.02 |
| % CV | 0.8% | 0.8% |
| N | 20 | 20 |

The expected inter-assay precision was 0.9-2.7% C. The CLS acceptable limit was less than 2.6% CV. The following demonstrates inter-assay precision determined by daily analysis of three levels of commercial quality control materials.

TABLE 9
Inter-Assay Precision

| | Level I | Level II |
|---|---|---|
| Mean (U/L) | 109.43 | 425.88 |
| SD | 1.88 | 5.84 |
| % CV | 1.7% | 1.4% |
| N | 10 | 10 |

Accuracy was assessed using Survey Validation Reference Material. Three levels of commercial quality control material were used for start-up. For within run quality control, Level I and Level II were run at the beginning and end of each load. The maximum load size was 100 specimens.

TABLE 10
Accuracy

| | CLS | Known | | Pass |
|---|---|---|---|---|
| | (U/L) | Mean | Recovery % | Yes/No |
| CAP C-B2013 CHM-08 | 213.800 | 213.000 | 100.376% | Yes |
| CAP C-A2013 CHM-10 | 391.300 | 386.000 | 101.373% | Yes |

*Acceptable recovery was between 90 and 110%

The LD linearity was evaluated and updated with each new shipment of reagent or every six months. The linear range of the assay was 6-1000 U/L. The maximum dilution that could be prepared was X16, extending the upper reporting limit to 16,000 U/L.

TABLE 11
Reference Range

| Age | Male U/L | Female U/L |
|---|---|---|
| 0-1 M | 125-735 | 145-765 |
| 1 M-1 yr | 170-450 | 190-420 |
| 1-3 yrs | 155-345 | 165-395 |
| 3-6 yrs | 155-345 | 135-345 |
| 6-9 yrs | 145-300 | 140-280 |
| 9-12 yrs | 120-325 | 120-260 |
| 12-15 yrs | 120-290 | 100-275 |
| 15-18 yrs | 105-235 | 105-230 |
| 18 years and older | 53-234 | 53-234 |

The manufacturer states no significant icteric interference up to an I index of 60. RBC contamination elevates results. The H index was set at 15. No significant lipemia interference up to an L index of 1000.

2. Results

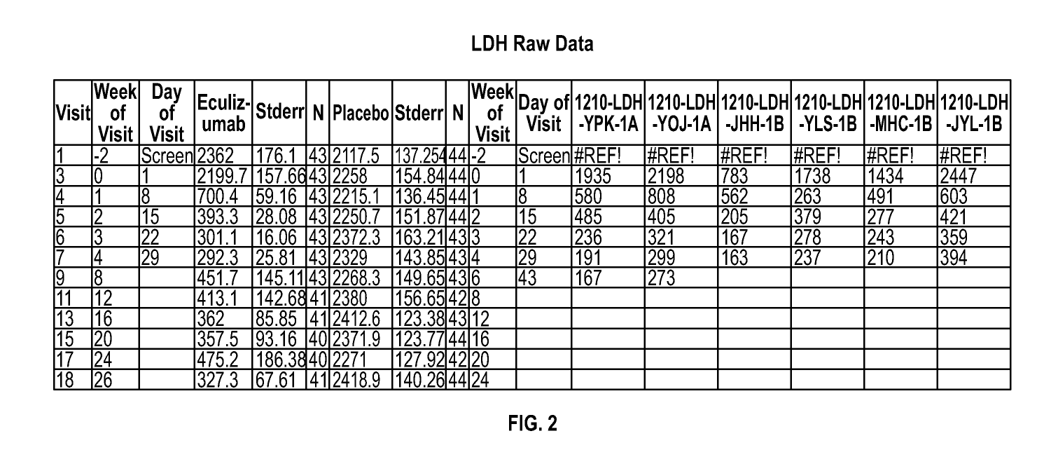

The resulting data reflects the reduction in LDH levels in patients (Cohorts 1a and 1b) through study week 6 and is shown in FIGS. 2-6.

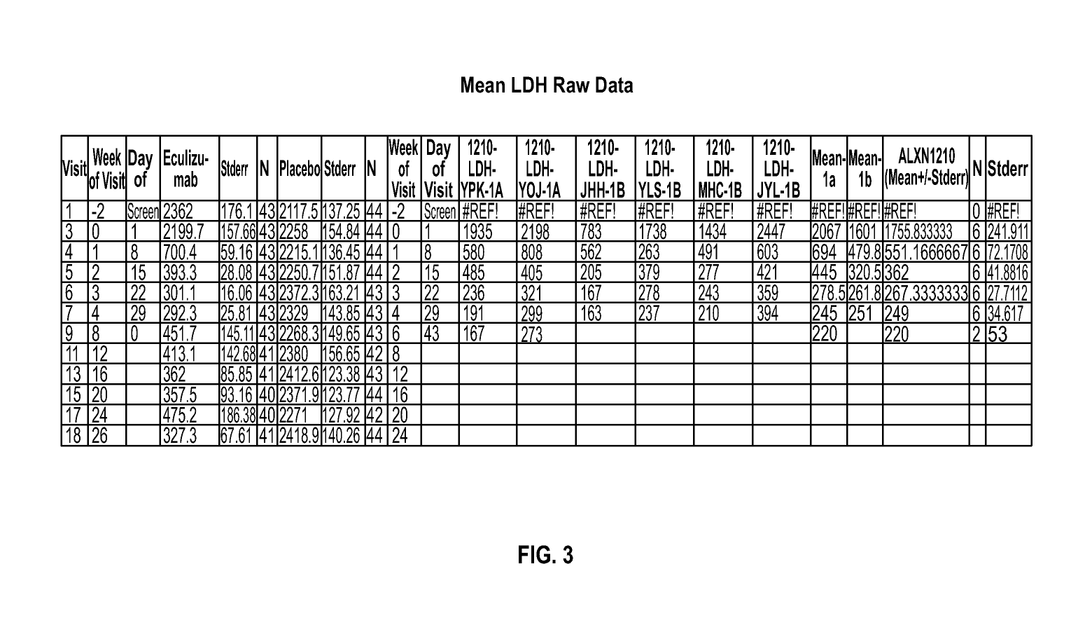

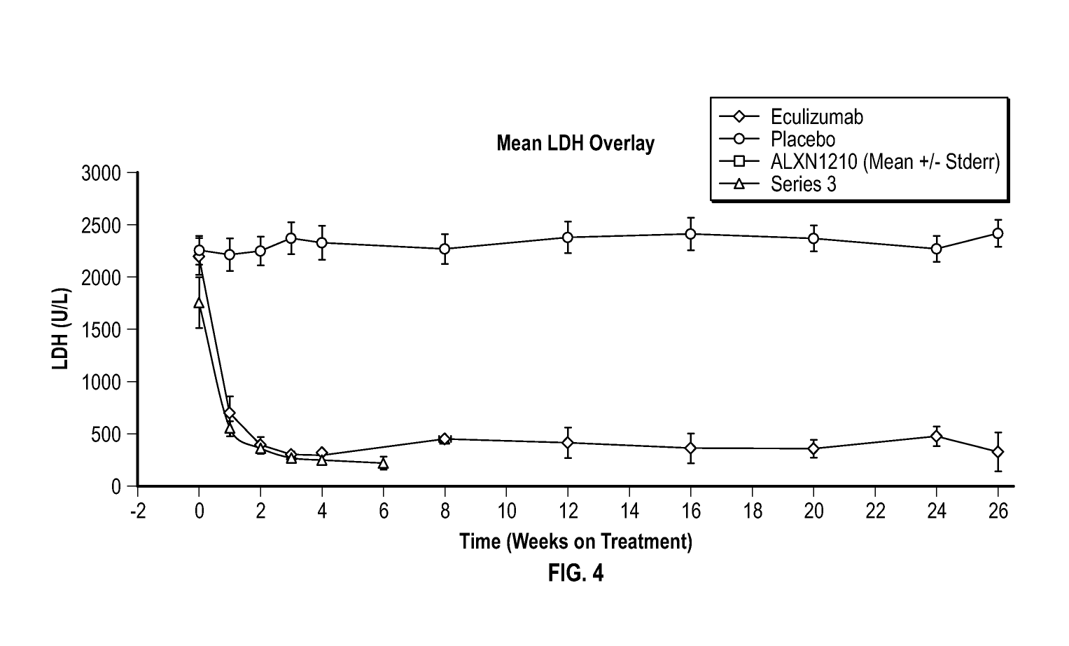

FIG. 2 is the raw LDH data for individual patients in Cohorts 1a and 1b after treatment with ALXN1210, as well as the raw LDH data for PNH patients after treatment with eculizumab (for comparative purposes). FIG. 3 is the raw mean LDH data for patients in Cohorts 1a and 1b after treatment with ALXN1210 (according to the protocol of Example 1), as well as the raw mean LDH data for PNH patients after treatment with eculizumab (for comparative purposes). FIG. 4 is the corresponding graph which depicts the mean LDH data for the patients in Cohorts 1a and 1b (treated with ALXN1210), compared to patients treated with eculizumab or a placebo. Table 12 summarizes the x-fold decreases in LDH levels for Cohort 1a, Cohort 1b, and both Cohorts on weeks 4 and 6 of the treatment regimens.

As indicated in FIGS. 3-4, the mean LDH level for Cohort 1a prior to treatment with ALXN1210 was 2067. After four weeks of the treatment regimen, the mean LDH level for the patients in Cohort 1a had dropped to 245 (resulting in an approximate 8.4 fold decrease compared to week 0) and after six weeks the mean LDH level dropped to 220 (resulting in an approximate 9.4 fold decrease compared to week 0). Similarly, the mean LDH level for Cohort 1b prior to treatment with ALXN1210 was 1601. After four weeks of the treatment regimen, the mean LDH level for the patients in Cohort 1b had dropped to 251 (resulting in an approximate 6.4 fold decrease compared to week 0). The mean LDH level for both Cohorts 1a and 1b prior to treatment with ALXN1210 was 1756. After four weeks of the treatment regimen, the mean LDH level for the patients in both cohorts had dropped to 249 (resulting in an approximate 7.1 fold decrease compared to week 0) and after six weeks the mean LDH level dropped to 220 (resulting in an approximate 8.0 fold decrease compared to week 0).

TABLE 12
Summary of LDH Level Decreases 4 and 6 Weeks Post-Treatment

| | Week 0 | Week 4 | Week 6 |
|---|---|---|---|
| | of Visit | of Visit | of Visit |
| Mean LDH | 2067 | 245 | 220 |
| level - | | *8.4 fold | *9.4 fold |
| Cohort 1a | | decrease from | decrease from |
| | | Week 0 to Week 4. | Week 0 to Week 6. |
| Mean LDH | 1601 | 251 | (Data Not Yet |
| level - | | *6.4 fold | Available) |
| Cohort 1b | | decrease from | |
| | | Week 0 to Week 4. | |
| Mean LDH | 1756 | 249 | 220 |
| level - | | *7.1 fold | *9.4 fold |
| Cohorts 1a | | decrease from | decrease from |
| and 1b | | Week 0 to Week 4. | Week 0 to Week 6. |

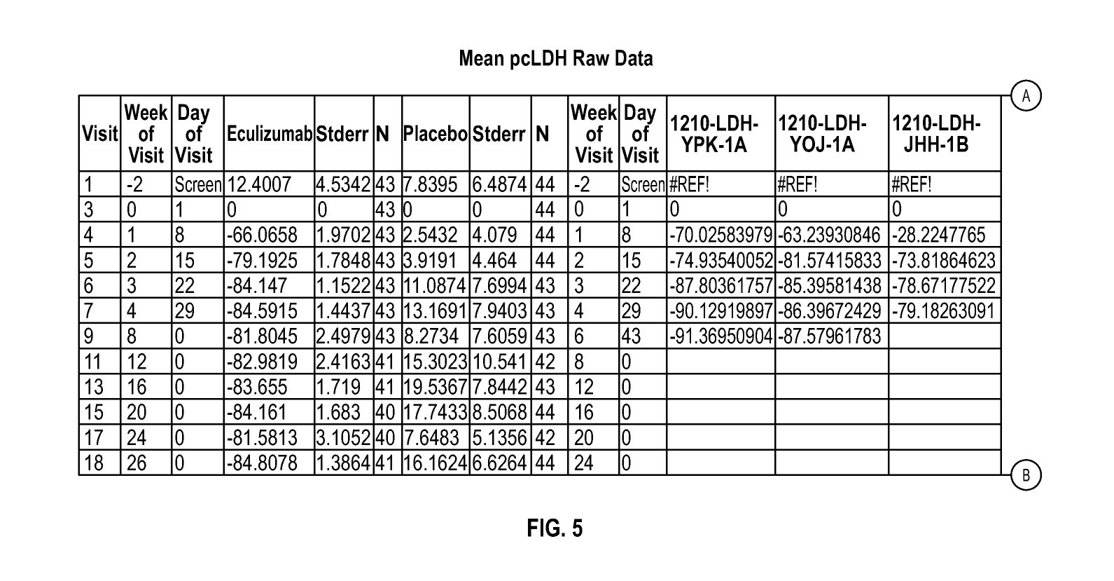

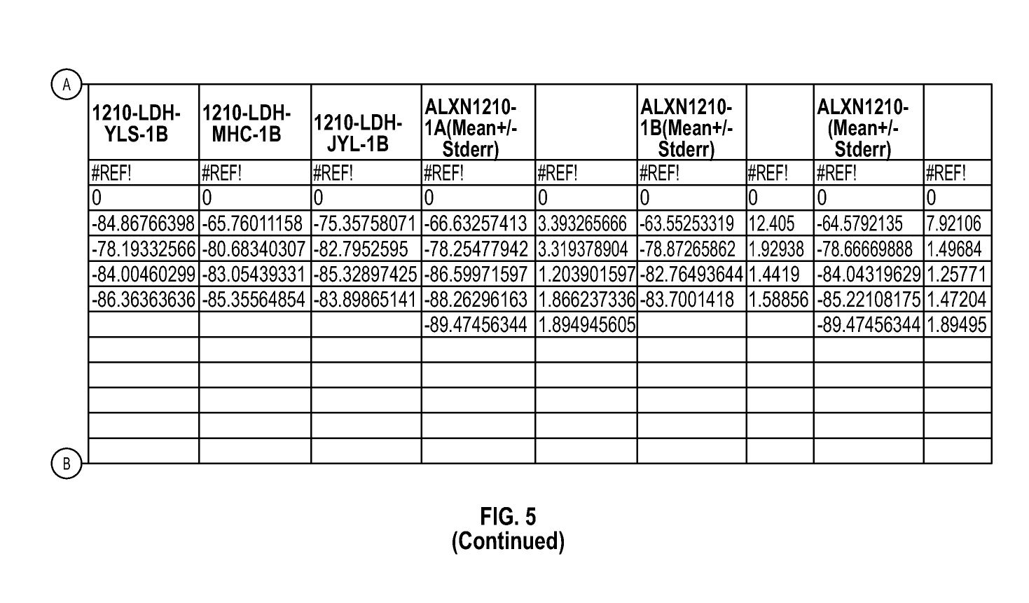

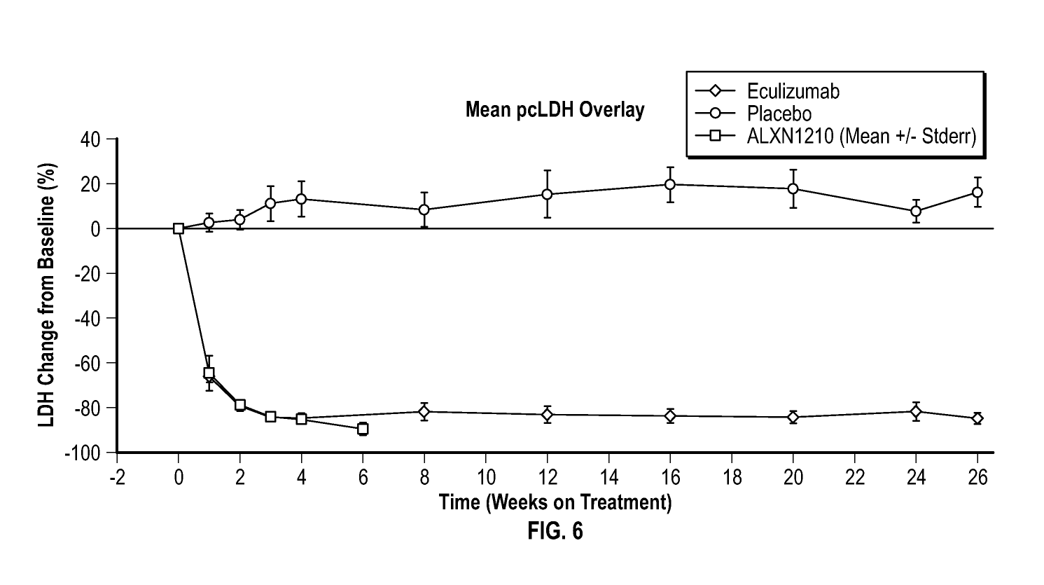

FIG. 5 is the raw mean LDH percentage change from baseline data for patients in Cohorts 1a and 1b (after treatment with ALXN1210 according to the protocol described in Example 1), as well as the raw mean LDH percentage change from baseline data for PNH patients after treatment with eculizumab (for comparative purposes). FIG. 6 is a graph which depicts the LDH percentage change from baseline for the patients in Cohorts 1a and 1b (treated with ALXN1210), compared to the LDH percentage change from baseline for patients treated with eculizumab or a placebo.

As indicated in FIGS. 5-6 and summarized in Table 13, the mean percent LDH change from baseline for Cohort 1a was approximately −88% after four weeks of treatment and approximately −89% after six weeks of treatment. The mean percent LDH change from baseline for Cohort 1b was approximately −84% after four weeks of treatment. The mean percent LDH change from baseline for both Cohorts 1a and 1b was approximately −85% after four weeks of treatment and approximately −89% after six weeks of treatment.

TABLE 13
Summary of LDH Change from Baseline
(%) - 4 and 6 Weeks Post-Treatment

| | Week 4 | Week 6 |
|---|---|---|
| | of Visit | of Visit |
| LDH Change from | −88% | −89% |
| Baseline (%) - | | |
| Cohort 1a | | |
| LDH Change from | −84% | (Data Not Yet |
| Baseline (%) - | | Available) |
| Cohort 1b | | |
| LDH Change from | −85% | −89% |
| Baseline (%) - | | |
| Cohorts 1a and 1b | | |

In summary, a significant decrease in mean LDH levels was observed for both Cohorts after 4 weeks of treatment (e.g., at least a 6-9 fold decrease) and for Cohort 1a after 6 weeks of treatment (e.g., at least a 9 fold decrease). The 6 week data is not yet available for Cohort 1b. Similarly, a significant percent decrease in LDH from baseline (e.g., at least 84%-89%) for both Cohorts was observed 4 weeks post-treatment. A significant percent decrease in LDH from baseline (e.g., at least 89%) for Cohort 1a was also observed after 6 weeks post-treatment. 6 week data not yet available for Cohort 1b.

### Example 3: Further Interim Results from Open-Label, Intrapatient, Dose-Escalation Study in PNH Patients

The following is a summary of interim data from an ongoing open-label, multiple-dose, multi-center intrapatient dose-escalation study conducted substantially according to the protocol described above in Example 1, which supplements the data described in Example 2.

In this interim analysis, two cohorts of patients 18 years of age or older were investigated. Patients in Cohort 1(C1) received either 400 or 600 mg induction doses, followed by a 900 mg maintenance dose every four weeks (i.e., q4w). Patients in Cohort 2 (C2) received 600 and 900 mg induction doses, followed by an 1800 mg maintenance dose every four weeks (i.e., q4w). The primary objective was to assess safety and tolerability of ALXN1210. The primary efficacy outcome was change from baseline in lactate dehydrogenase (LDH) level. Other endpoints included change in blood transfusions and in hematologic parameters related to PNH.

A total of 13 patients enrolled in the trial. The baseline demographics and disease characteristics of the patients are set forth in Table 14.

TABLE 14
Baseline Demographics and Disease Characteristics

| | | Cohort 1 | Cohort 2 | Overall |
|---|---|---|---|---|
| | | n = 6 | n = 7 | n = 13 |
| Race | | | | |
| Asian | n (%) | 6 (100) | 6 (86) | 12 (92) |
| White/Caucasian` | n (%) | 0 | 1 (14) | 1 (8) |
| Female Gender | n (%) | 2 (33) | 5 (71) | 7 (54) |
| Age at First Infusion | Mean (SD) | 41.1 (10.86) | 43.6 (13.49) | 42.4 (11.91) |
| (Years) | Median | 43.6 | 40.6 | 41.5 |
| | Min, Max | 24.5, 55.9 | 24.9, 62.3 | 24.5, 62.3 |
| lactate dehydrogenase | Mean (SD) | 1709.9 (582.10) | 1532.7 (355.05) | 1614.5 (461.16) |
| LDH (U/L) | | | | |
| | Median | 1744.8 | 1424.3 | 1541.0 |
| | Min, Max | 779.3-2392.7 | 1048.7-2057.7 | 779.3-2392.7 |
| lactate dehydrogenase | Mean (SD) | 7.3 (2.49) | 6.6 (1.52) | 6.9 (197) |
| LDH (xULN)* | Median | 7.5 | 6.1 | 6.6 |
| [xULN = multiples (fold) of | Min, Max | 3.3-10.2 | 4.5-8.8 | 3.3-10.2 |
| the upper limit of normal] | | | | |
| Total PNH RBC clone size | Mean (SD) | 56.7 (15.81) | 51.3 (23.39) | 53.8 (19.63) |
| (%) | Median | 54.6 | 38.1 | 51.3 |
| | Min, Max | 39.5-84.9 | 29.4-90.5 | 29.4-90.5 |
| PNH Granulocyte Clone | Mean (SD) | 87.7 (7.56) | 86.4 (11.06) | 87.0 (9.24) |
| Size | Median | 87.9 | 89.1 | 89.1 |
| | Min, Max | 76.4-98.4 | 69.4-99.4 | 69.4-99.4 |
| Patients with Transfusion | n (%) | 2 (33) | 3 (43) | 5 (38) |
| in Year Prior to Enrollment | | | | |
| Patients with Transfusion | n (%) | 2 (33) | 3 (43) | 5 (38) |
| within 1 Year Prior to First | | [7 | [4 transfusions, | [11 transfusions, |
| ALXN1210 Dose | | transfusions, | 8 units] | 24 units] |
| | | 16 units] | | |
| Patients with Transfusion | n (%) | 1 (17) | 0 | 1 (8) |
| Since First ALXN1210 | | [2 | | [2 transfusions, |
| Dose* | | transfusions, 4 | | 4 units] |
| | | units] | | |

*The median (range) follow-up time on ALXN1210 treatment was 4.7 (4.6-4.7 months) in Cohort 1 and 2.8 (2.7-3.7 months) in Cohort 2.

Statistical Analysis: Efficacy and safety endpoints were analyzed descriptively. For LDH levels, baseline was defined as the average of values at screening, prior to the first ALXN1210 infusion. For hemoglobin levels, baseline was defined as the most recent value prior to the first ALXN1210 infusion.

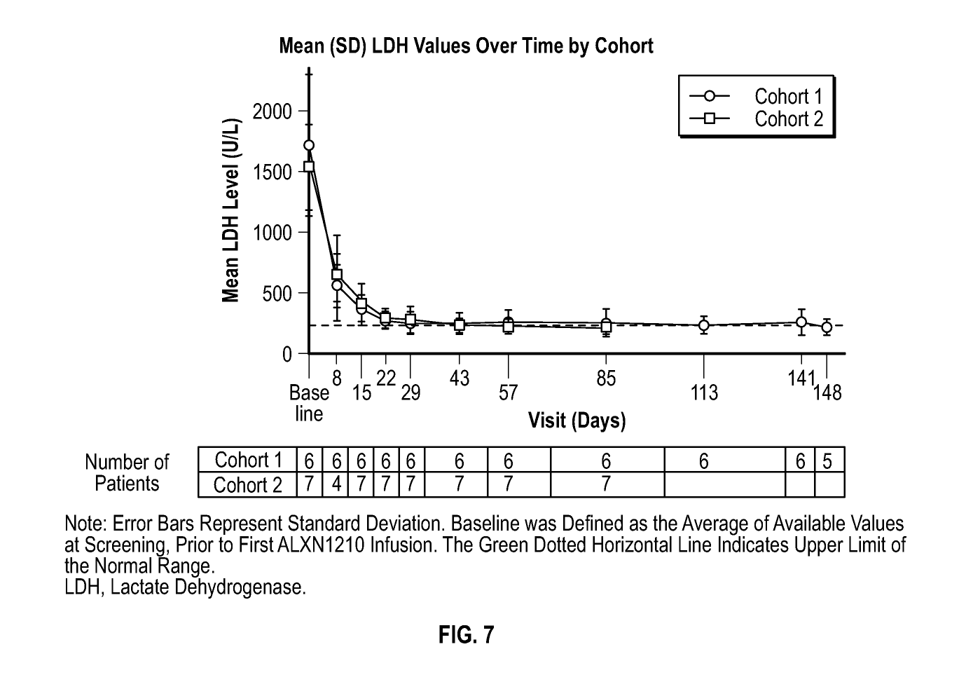

The median (range) duration of exposure to ALXN1210 was 4.7 (4.6-4.7) months for C1 (n=6) and 2.8 (2.7-3.7) months for C2 (n=7). All patients showed rapid reductions in LDH levels, which were observed at the first evaluable time point (Day 8). Decreases in LDH were sustained over 5 month dose intervals, as shown by mean reductions from baseline in LDH of 85.6% in C1 on Day 113 and 83.8% in C2 on Day 57 (FIG. 7). At the last evaluable time point, the mean percentage reduction from baseline in LDH level was 85.4% in Cohort 1 (on day 148) and 86.0% in Cohort 2 (on day 85) (FIG. 7). Baseline is defined as the average of all available values prior to first ALXN1210 infusion. Mean hemoglobin levels were improved or stable in both cohorts. Among 5 patients with ≥1 transfusion in the year prior to treatment (two patients in C1; three patients in C2), only one patient (in C1, who had received 12 units of packed red blood cells in the prior 6 months) received a transfusion (2 units) while on ALXN1210. This 45-year-old male patient (Cohort 1) with a history of aplastic anemia had received 12 units of packed red blood cells in the prior 6 months before study entry. He received 2 units while being treated with ALXN1210 and had no serious TEAEs. Multiple doses of ALXN1210 resulted in no severe adverse events, no infusion site reactions, and no drug discontinuations or adverse events leading to withdrawals. The most common treatment-emergent adverse event (TEAE) was headache (in 2 patients). TEAEs during ALXN1210 treatment occurred in 5 patients (83.3%) in Cohort 1 and 6 patients (85.7%) in Cohort 2 (Table 15). The most common TEAEs were headache and upper respiratory tract infection; each occurred in 3 patients (23.1%). Multiple doses of ALXN1210 resulted in no serious AEs, withdrawals from the study, or deaths. Investigators judged 79.5% of the TEAEs to be unrelated to ALXN1210 treatment. All related TEAEs resolved during ongoing ALXN1210 treatment. Treatment-Emergent Adverse Events by Cohort are summarized in Table 15.

TABLE 15
Treatment-Emergent Events by Cohort

| | | | Overall |
|---|---|---|---|
| Parameter | Cohort 1 (n = 6) | Cohort 2 (n = 7) | (N = 13) |
| Patients with | 0 | 0 | 0 |
| serious TEAEs, n | | | |
| (%) | | | |
| Patients with | 5 (83) | 6 (86) | 11 (85) |
| TEAEs, n (%) | | | |
| Patients with | 1 (17) | 2 (29) | 3 (23) |
| related TEAs, n (%) | | | |
| Anemia | 1 (17)^{B} | 0 | 1 (8) |
| Atrial Flutter | 0 | 1 (14)^{C} | 1 (8) |
| Headache | 0 | 1 (14)^{D} | 1 (8) |
| Pain | 0 | 1 (14)^{E} | 1 (8) |

^{A}Judged by the investigator to be possibly related to ALXN1210 treatment.
^{B}Two events occurred on days 56 and 140 (both grade 3) and both resolved.
^{C}Occurred on day 72 (grade 3) and resolved.
^{D}Four events occurred on days 1, 15, 29, and 55 (all grade 2) and all resolved.
^{E}Occurred on day 57 (grade 1) and resolved.

Preliminary Pharmacokinetic Analysis: Available preliminary ALXN1210 data have demonstrated extended t½ and dose- and time-linear pharmacokinetics. In the ongoing study, ALXN1210 serum concentrations are approaching steady state.

This study is the first to demonstrate the efficacy and safety of ALXN1210 in patients with PNH. ALXN1210 treatment resulted in rapid reductions in LDH levels in 100% of patients (a direct measure of complement-mediated hemolysis), which were sustained through 5 monthly dosing intervals, consistent with the extended t½ of ALXN1210. Treatment with ALXN1210 resulted in rapid decreases in LDH levels in all patients by the first measured time point (day 8). At the last evaluable time point, the mean percentage reduction from baseline in LDH level was 85.4% in Cohort 1 (on day 148) and 86.0% in Cohort 2 (on day 85). LDH reductions were sustained through all monthly dosing intervals in all patients.

There was a notable decrease in the need for blood transfusions. Four of 5 patients who had received transfusions in the 12 months prior to treatment did not require transfusion while on ALXN1210; 1 patient received a transfusion during treatment, but required only 2 units while on ALXN1210 versus 12 units in the 6 months prior to starting ALXN1210.

Overall, these preliminary LDH data suggest that rapid, complete, and sustained complement inhibition with ALXN1210 results in highly effective blockade of intravascular hemolysis in monthly dosing intervals.

### Example 4: Further Interim Results from Open-Label, Intrapatient, Dose-Escalation Study in PNH Patients

The following is a summary of further interim data from an ongoing open-label, multiple-dose, multi-center intrapatient dose-escalation study conducted substantially according to the protocol described above in Example 1, which supplements the data described in Examples 2-3.

The mean (SD) duration of exposure to ALXN1210 was 5.6 (0.03) months, the median (range) was 5.6 months and the minimum/maximum were 5.5 and 5.6, respectively, for C1 (n=6). The mean (SD) duration of exposure to ALXN1210 was 3.4 (0.46) months, the median (range) was 3.7 months and the minimum/maximum were 2.7 and 3.8, respectively, for C1 (n=7). The overall mean (SD) duration of exposure to ALXN1210 for both cohorts (n=13) was 4.4 (1.15) months, the median (range) was 3.8 months and the min./max. were 2.7 and 5.6, respectively.

ALXN1210 continues to show an acceptable safety profile up to 1800 mg. The treatment-emergent adverse events by cohort remain as set forth in Table 15 of Example 3 (i.e., no reported serious TEAE's, most common TEAE was headache, and most TEAE's are unrelated to treatment).

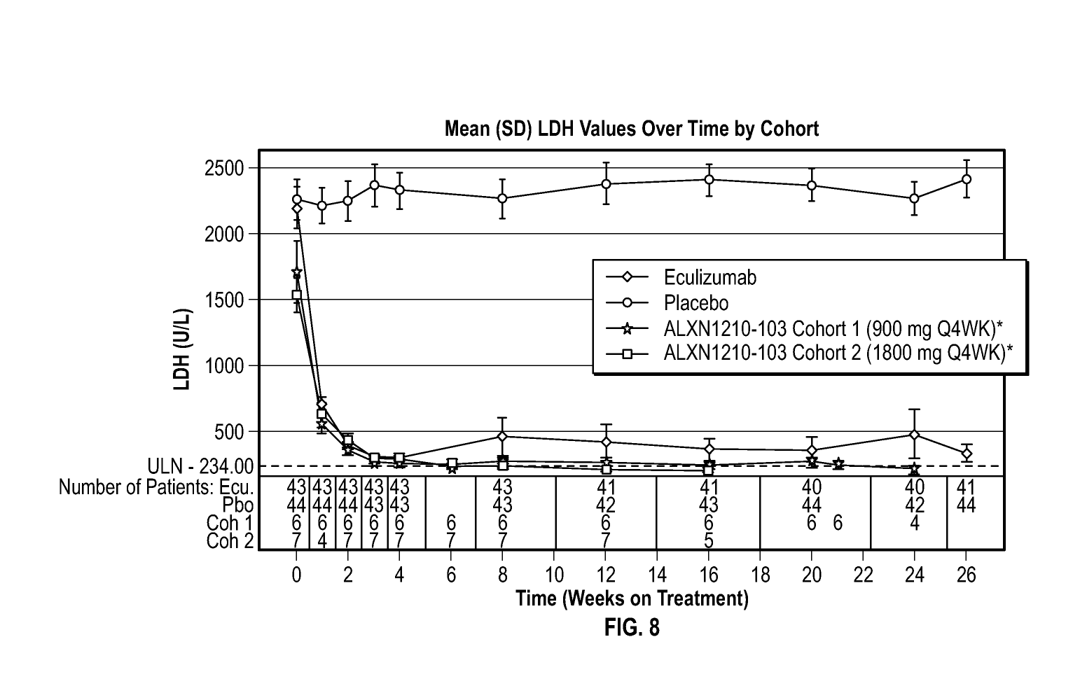

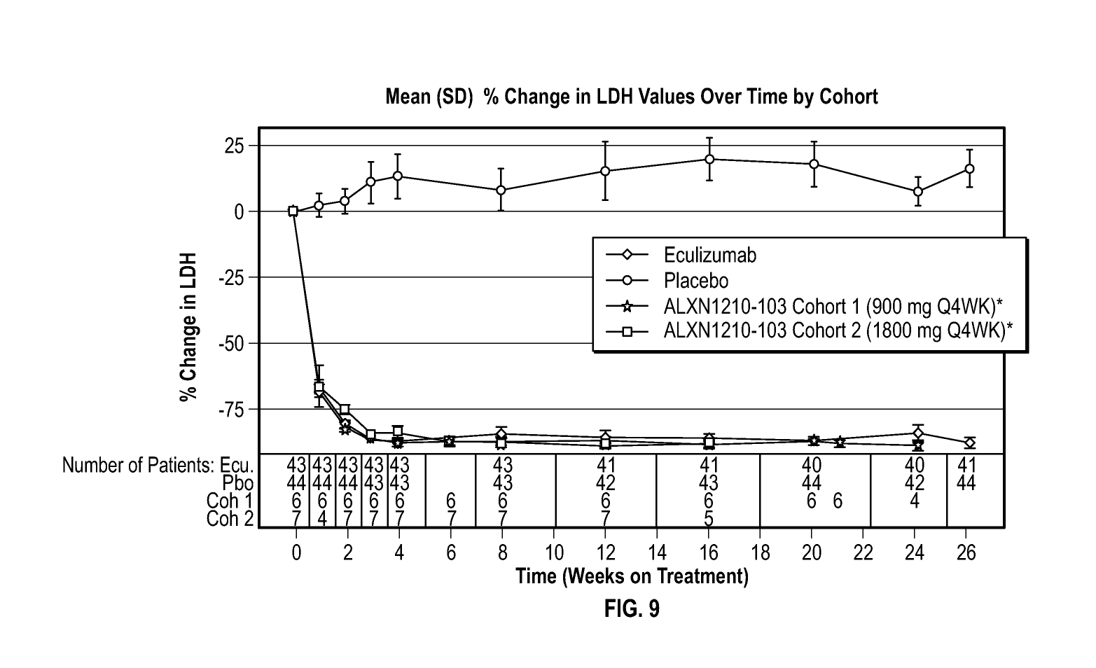

FIG. 8 is a graph which depicts the mean LDH over time for patients in Cohorts 1 and 2 (treated with ALXN1210), compared to the LDH over time for patients treated with eculizumab or a placebo. FIG. 9 is a graph which depicts the mean percent change in LDH over time for patients in Cohorts 1 and 2 (treated with ALXN1210), compared to the mean percent change in LDH over time for patients treated with eculizumab or a placebo. As shown in FIGS. 8 and 9, ALXN1210 treatment resulted in rapid and sustained reductions in LDH levels in 100% of patients (a direct measure of complement-mediated hemolysis). Treatment with ALXN1210 resulted in rapid decreases in LDH levels in all patients by the first measured time point (day 8).

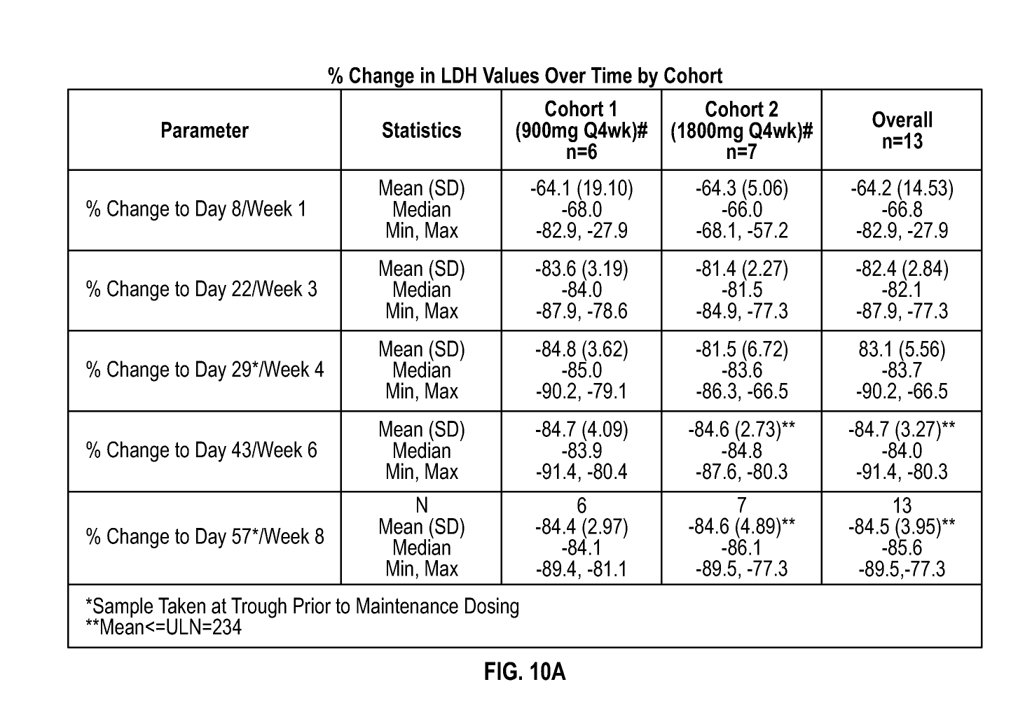

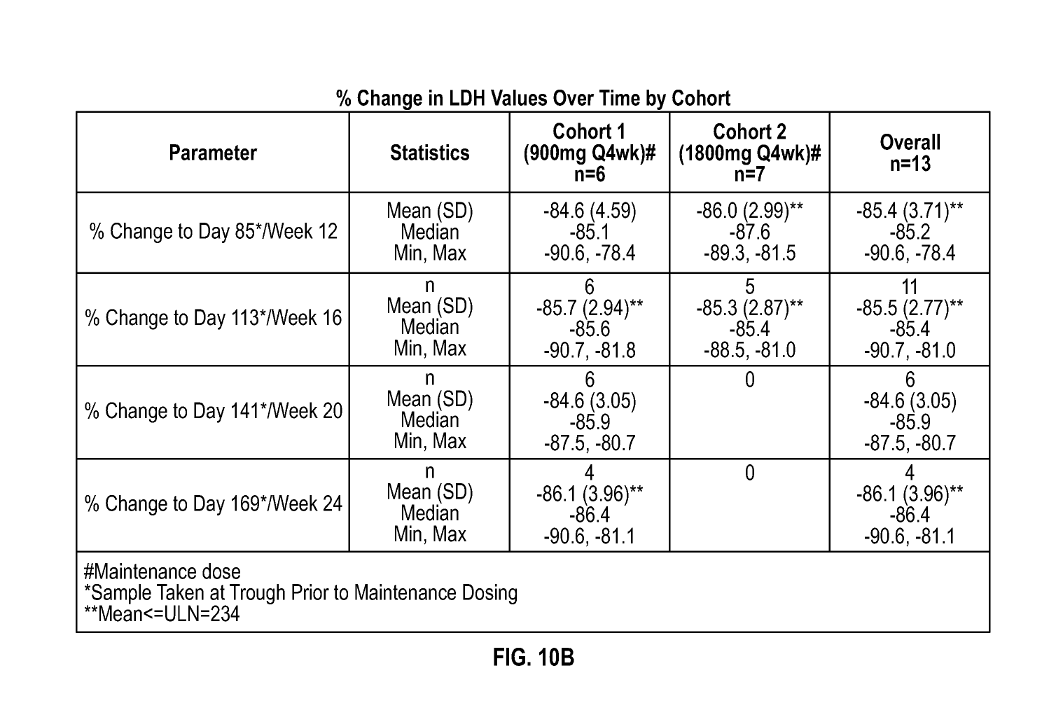

FIG. 10A is the raw mean, median, and minimum/maximum percentage change in LDH levels from baseline data for patients in Cohorts 1 and 2 after treatment with ALXN1210 from Week 1 through Week 8. FIG. 10B is the raw mean, median, and minimum/maximum percentage change in LDH levels from baseline data for patients in Cohorts 1 and 2 after treatment with ALXN1210 from Week 12 through Week 24. As shown in FIGS. 10A and 10B, all patients had a 40% reduction in LDH by Day 8 and the overall mean reduction on Day 22 was −82.4% (−83.6%, −81.4% for cohorts 1,2 respectively). As shown in FIGS. 10A and 10B, at the last evaluable time point, the mean percentage reduction from baseline in LDH level was 86.1% in Cohort 1 (on day 169) and 85.3% in Cohort 2 (on day 113). LDH reductions were sustained through all monthly dosing intervals in all patients. These preliminary results show a rapid and sustained LDH reduction in response to ALXN1210. The magnitude of LDH reduction (˜85%) was similar between Cohort 1 (900 mg maintenance dose) and Cohort 2 (1800 mg maintenance dose).

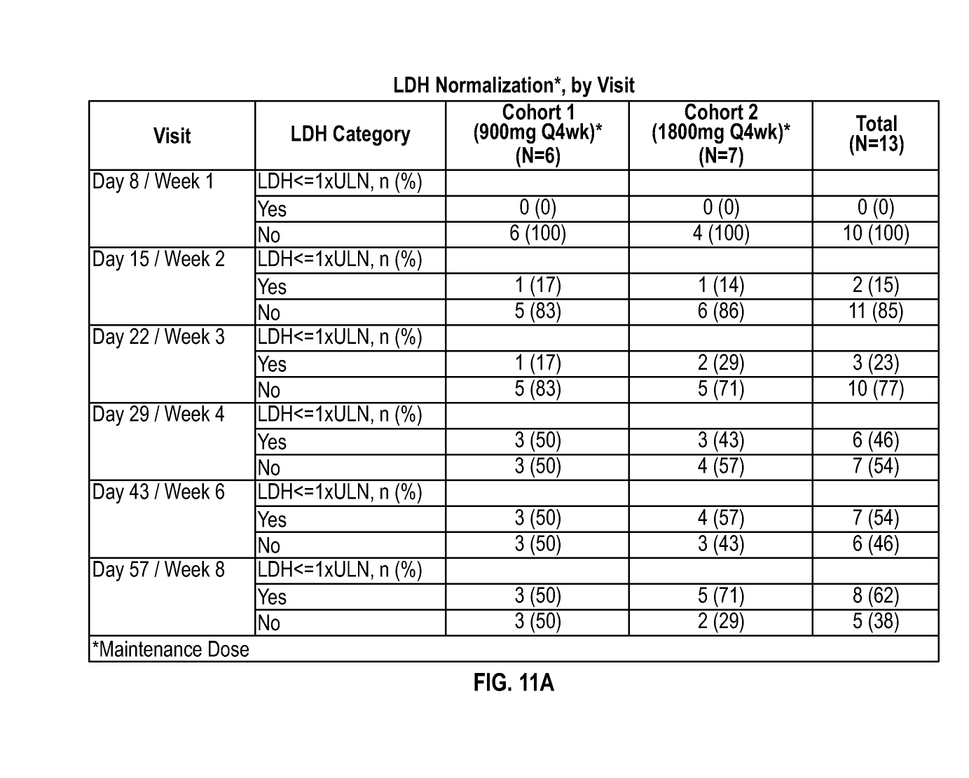

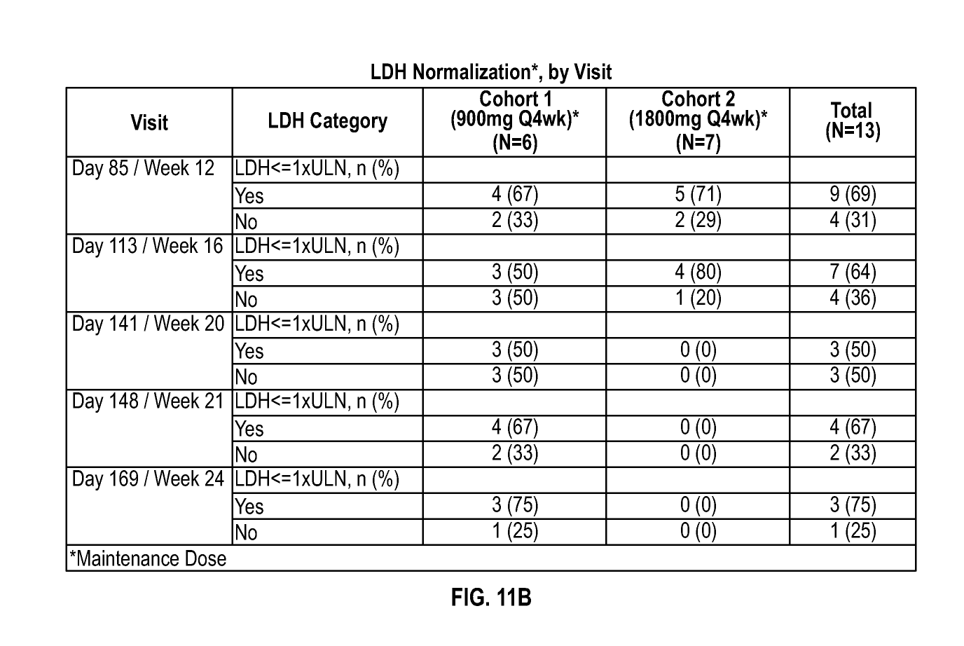

FIG. 11A is the raw mean LDH normalization data for patients in Cohorts 1 and 2 after treatment with ALXN1210 from Week 1 through Week 8. FIG. 11B is the raw mean LDH normalization data for patients in Cohorts 1 and 2 after treatment with ALXN1210 from Week 12 through Week 24.

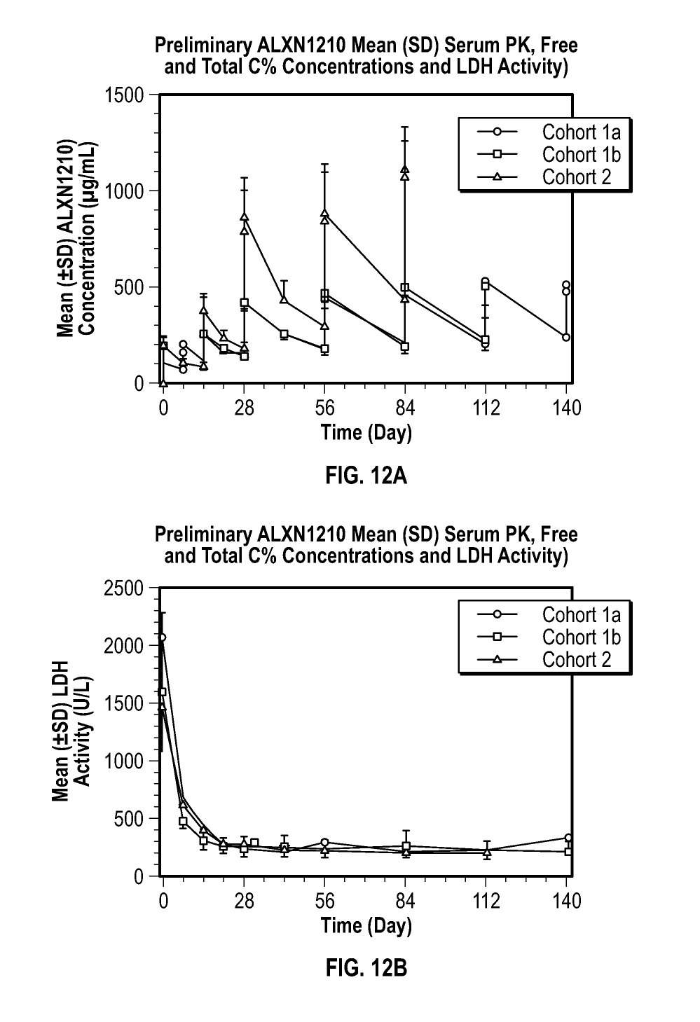

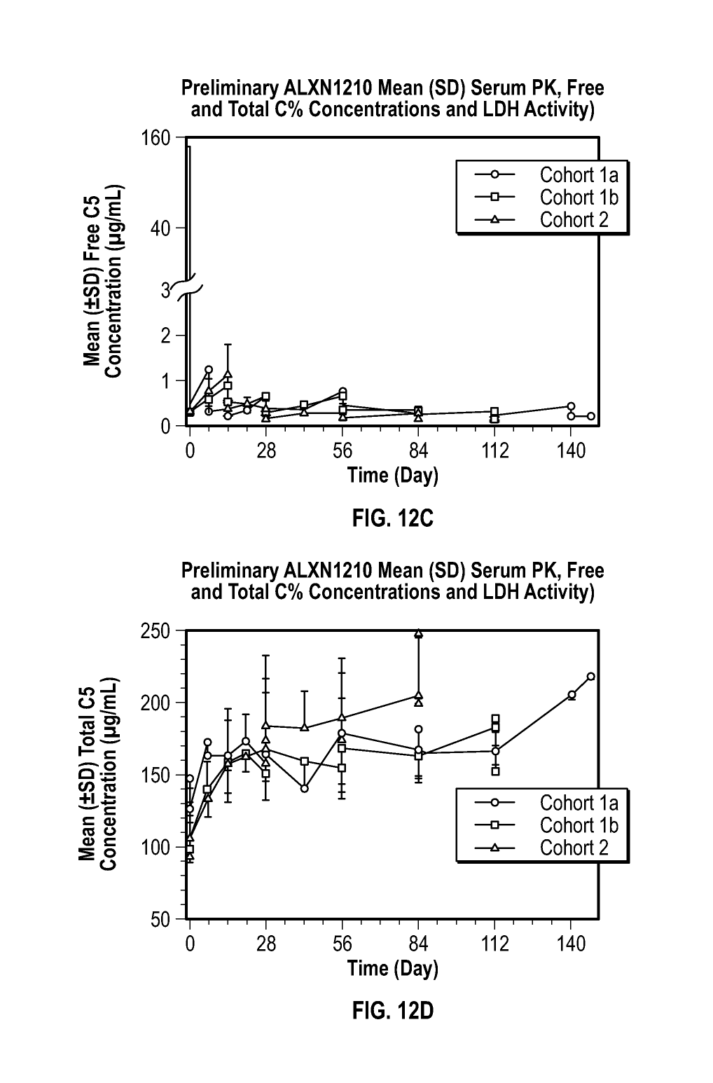

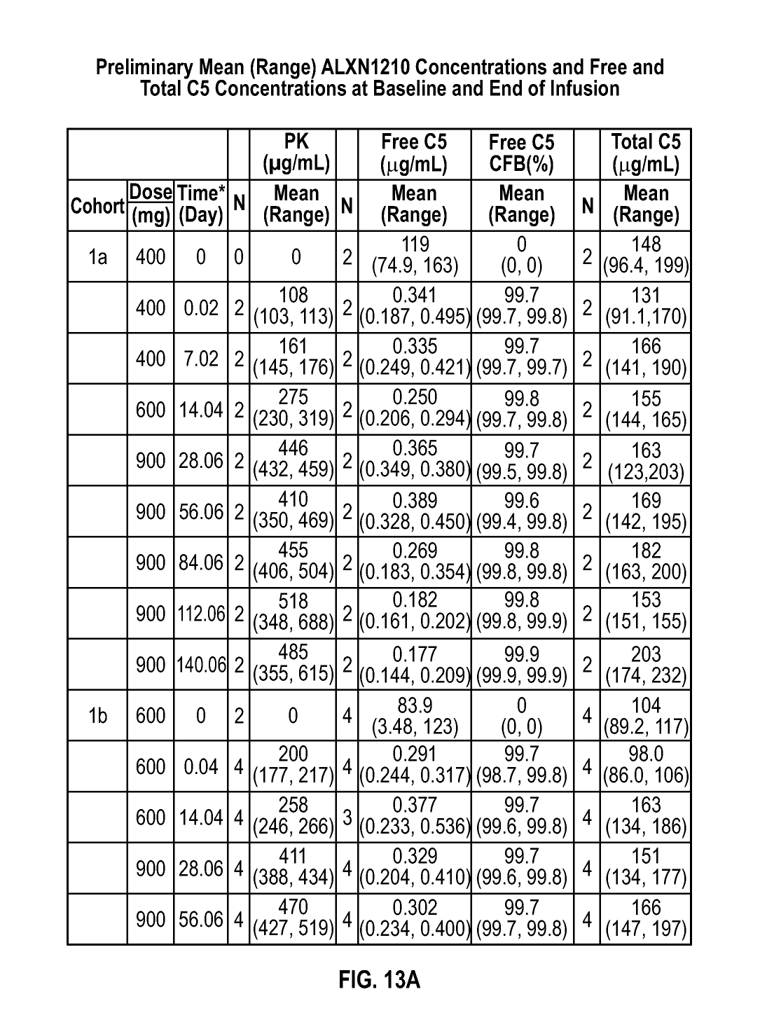

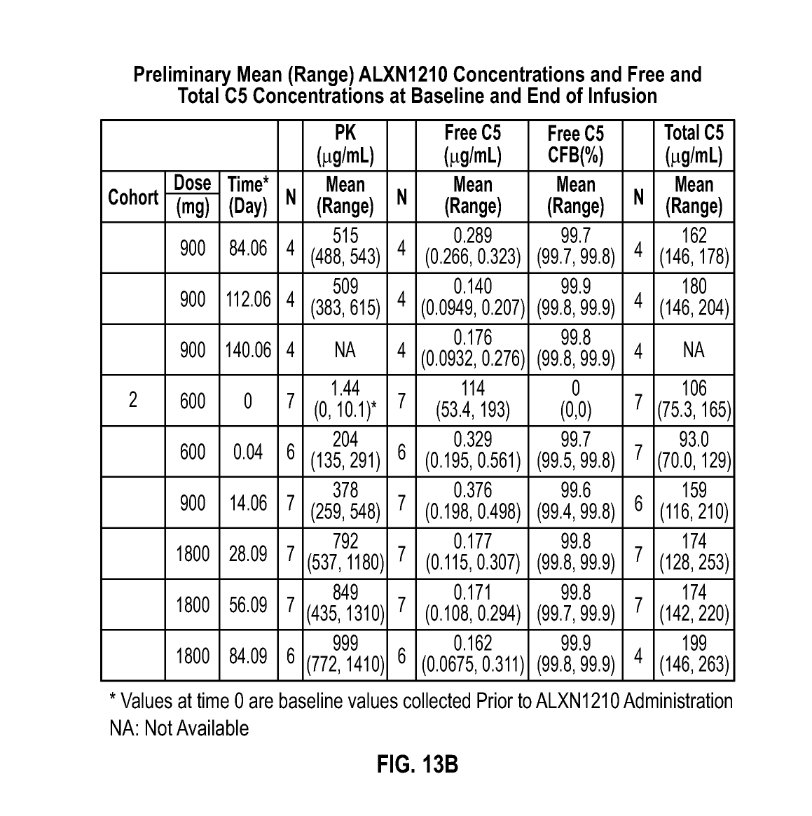

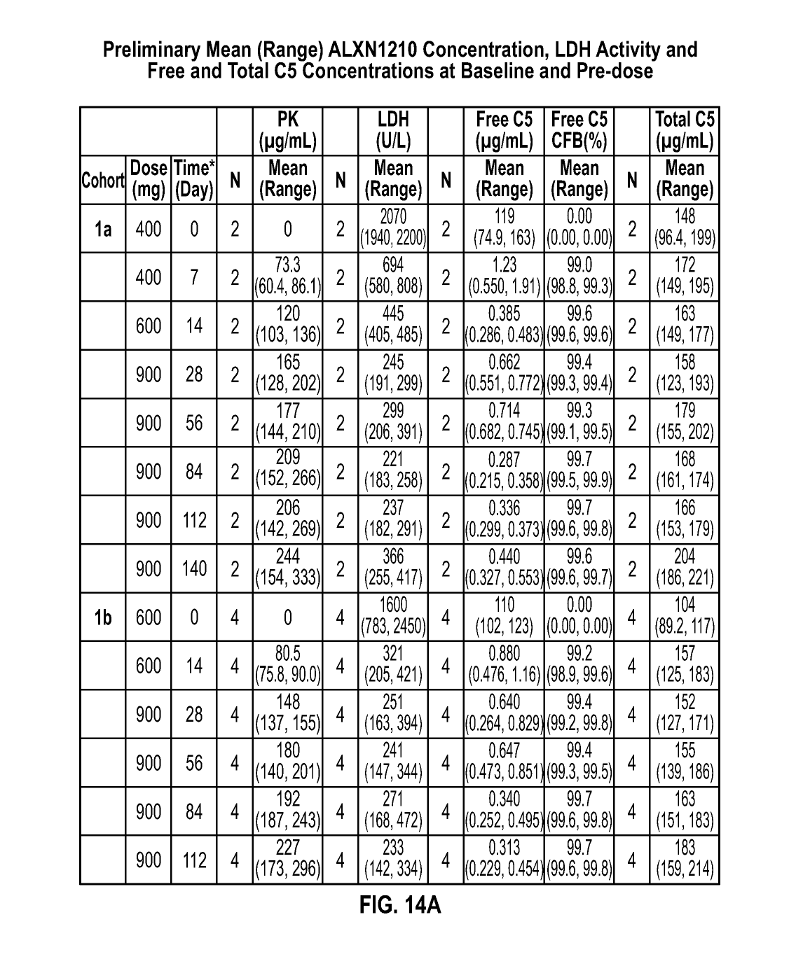

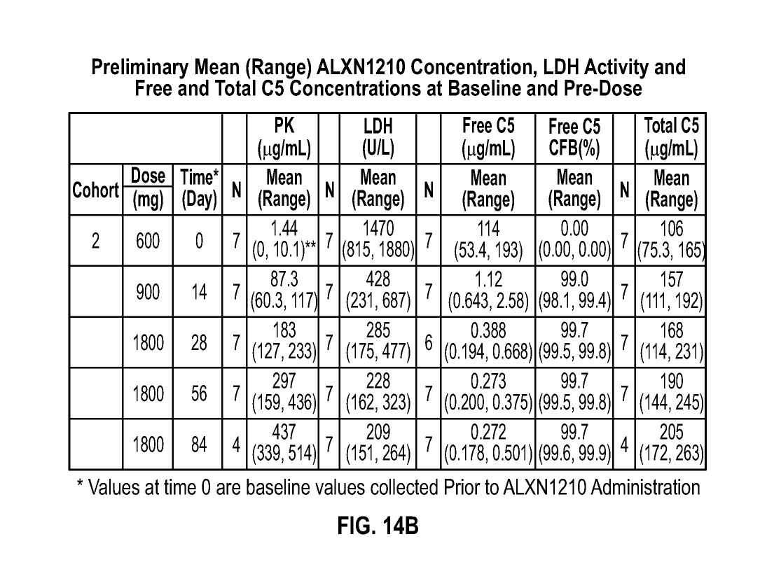

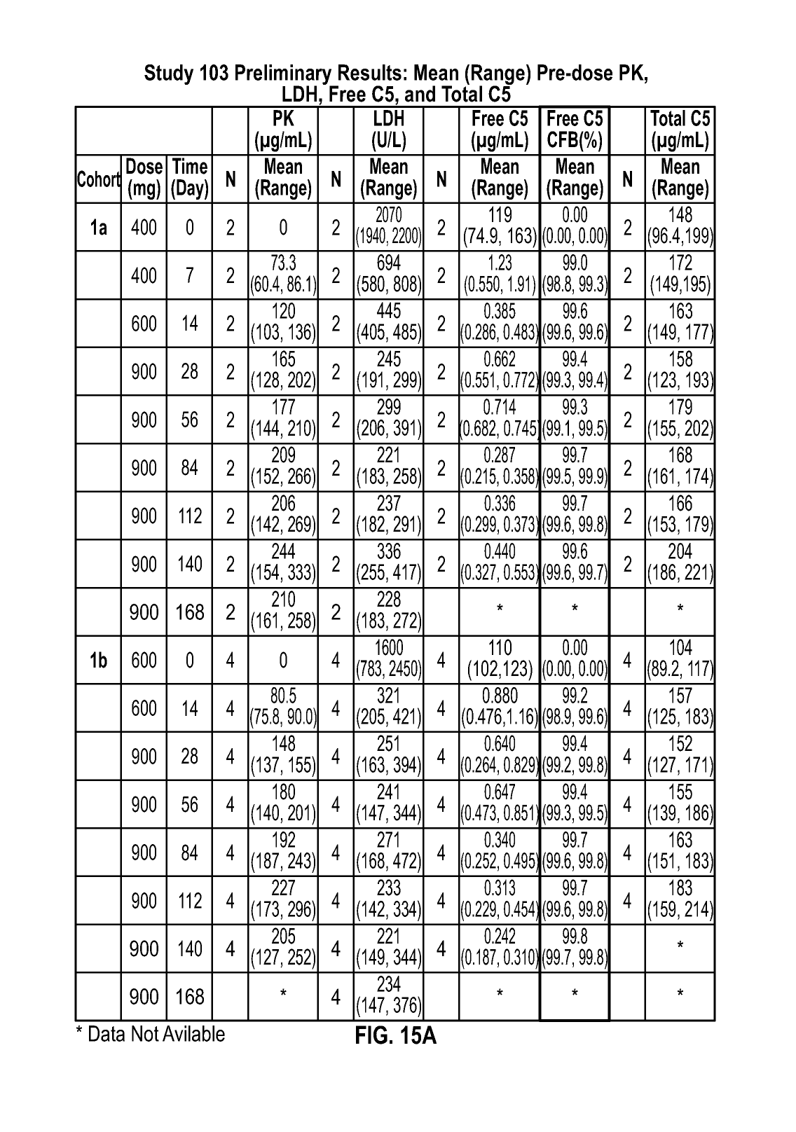

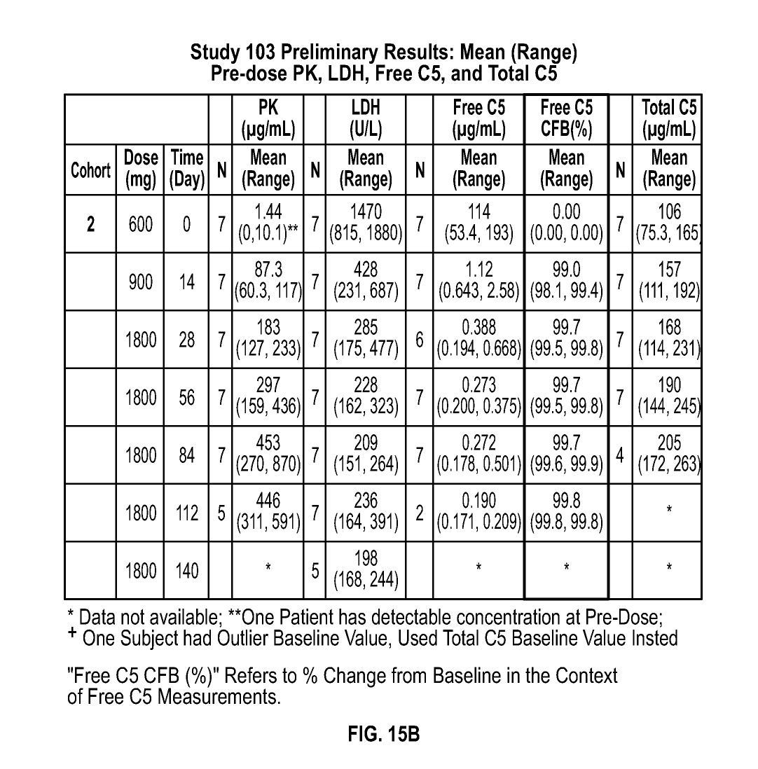

FIG. 12 displays preliminary serum PK, free and total C5 concentrations, and LDH activity following multiple dose administration in PNH patients. FIGS. 13A-13B and 14A-14B summarize the preliminary mean (range) ALXN1210 concentration, LDH activity and free and total concentrations at EOI and pre-dose. Following ALXN1210 multiple-dose administration in PNH patients, immediate, complete and sustained terminal complement inhibition was achieved, as evidenced by reduced serum free C5 by EOI and at predose, respectively. In PNH patients receiving assorted regimen of ALXN1210, the preliminary range of mean % CFB in free C5 concentrations was 99.6 to 99.8%, 99.7 to 99.9% and 99.6 to 99.9% at EOI for Cohorts 1a, 1b and 2, respectively. In PNH patients receiving assorted regimen of ALXN1210, the preliminary range of mean percent change from baseline % CFB in free C5 concentrations was 99.6 to 99.9%, 99.2 to 99.7% and 99.0 to 99.1% at predose for Cohorts 1a, 1b and 2, respectively. See column labelled Free C5 CFB (%) in FIGS. 15A and 15B. “% CFB” refers to percent change from baseline in the context of free C5 measurements.

FIGS. 15A-15B show preliminary mean (range) pre-dose PK, LDH, free C5, percent change from baseline in free C5, and total C5 at additional time points. As shown in FIGS. 15A-15B, the serum ALXN1210 concentration (mg/mL) data in patients with PNH over the studied dose regimens of 900 mg to 1800 Q4W was ≥100 μg/mL in all patients when given with appropriate loading doses. In addition, the serum free C5 concentration data in patients with PNH over the studied dose regimens of 900 mg to 1800 Q4W showed >99% reduction from baseline, thus ensuring immediate, complete and sustained complement inhibition in all patients when given with appropriate loading doses. The serum free C5 suppression correlated well with LDH response.

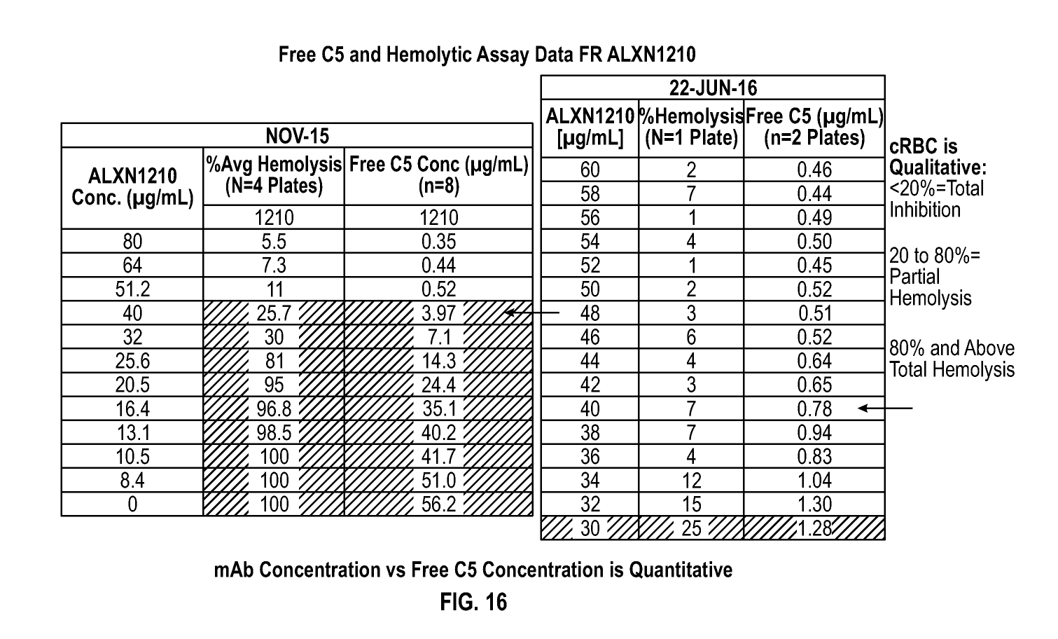

FIG. 16 sets forth the free C5 and hemolytic assay data. As shown in FIG. 16, total inhibition of cRBC hemolytic activity achieved at concentrations expected at studied dose regimens of 900 mg to 1800 mg.

These preliminary LDH data continue to suggest that rapid, complete, and sustained complement inhibition with ALXN1210 results in highly effective blockade of intravascular hemolysis in monthly dosing intervals.

### Example 5: Further Interim Results from Open-Label, Intrapatient, Dose-Escalation Study in PNH Patients

The following is a summary of further interim data from an ongoing open-label, multiple-dose, multi-center intrapatient dose-escalation study conducted substantially according to the protocol described above in Example 1, which supplements the data described in Examples 2-4.

ALXN1210-PNH-103 is a Phase 1/2, multicenter, open-label, intrapatient dose-escalation study (NCT02598583), evaluating the safety, tolerability, and efficacy of two intravenous (IV) maintenance dosing regimens of ALXN1210 in patients ≥18 y with paroxysmal nocturnal hemoglobinuria (PNH) who were naïve to complement inhibitor therapy.

In this interim analysis, 6 patients in Cohort 1 (C1) received either 400- or 600-mg IV induction doses, followed by a 900-mg maintenance dose q4w; 7 pts in Cohort 2 (C2) received 600- and 900-mg induction doses, followed by an 1800-mg maintenance dose once every four weeks (q4w) for up to 24 weeks. The primary efficacy outcome was change in complement-mediated hemolysis as measured by LDH level. Other endpoints included changes in hematologic parameters, blood transfusions, FACIT-Fatigue scores, and pharmacokinetic (PK) parameters.

A total of 13 patients consented and enrolled. The baseline demographics and disease characteristics of the patients are set forth in Table 16.

TABLE 16
Baseline Demographics and Disease Characteristics

| | | Cohort 1 | Cohort 2 | |
|---|---|---|---|---|
| | | (900 mg) | (1800 mg) | Overall |
| | | n = 6 | n = 7 | N = 13 |
| Race | | | | |
| Asian | n (%) | 6 (100) | 6 (86) | 12 (92) |
| White/Caucasian | n (%) | 0 | 1 (14) | 1 (8) |
| Female Gender | n (%) | 2 (33) | 5 (71) | 7 (54) |
| Age at First | Mean (SD) | 41.1 (10.86) | 43.6 (13.49) | 42.4 (11.91) |
| Infusion (Years) | Median | 43.6 | 40.6 | 41.5 |
| | Min, Max | 24.5, 55.9 | 24.9, 62.3 | 24.5, 62.3 |
| LDH (U/L) | Mean (SD) | 1709.9 (582.10) | 1532.7 (355.05) | 1614.5 (461.16) |
| | Median | 1744.8 | 1424.3 | 1541.0 |
| | Min, Max | 779.3, 2392.7 | 1048.7, 2057.7 | 779.3, 2392.7 |
| LDH (xULN) | Mean (SD) | 7.3 (2.49) | 6.6 (1.52) | 6.9 (1.97) |
| | Median | 7.5 | 6.1 | 6.6 |
| | Min, Max | 3.3, 10.2 | 4.5, 8.8 | 3.3, 10.2 |
| PNH granulocyte | Mean (SD) | 90.0 (6.98) | 91.1 (6.51) | 90.6 (6.47) |
| clone size^{a} (%) | Median | 88.9 | 90.6 | 90.6 |
| | Min, Max | 81.4, 99.8 | 80.6, 99.4 | 80.6, 99.8 |
| Patients with | n (%) | 2 (33) | 3 (43) | 5 (38) |
| Transfusion in | | | | |
| Year Prior to | | | | |
| Enrollment | | | | |

^{a}CD24 and FLAER-negative cells.
FLAER, fluorescein-labeled proaerolysin;
LDH, lactate dehydrogenase;
PNH, paroxysmal nocturnal hemoglobinuria;
SD, standard deviation;
xULN, multiples (fold) of the upper limit of normal.

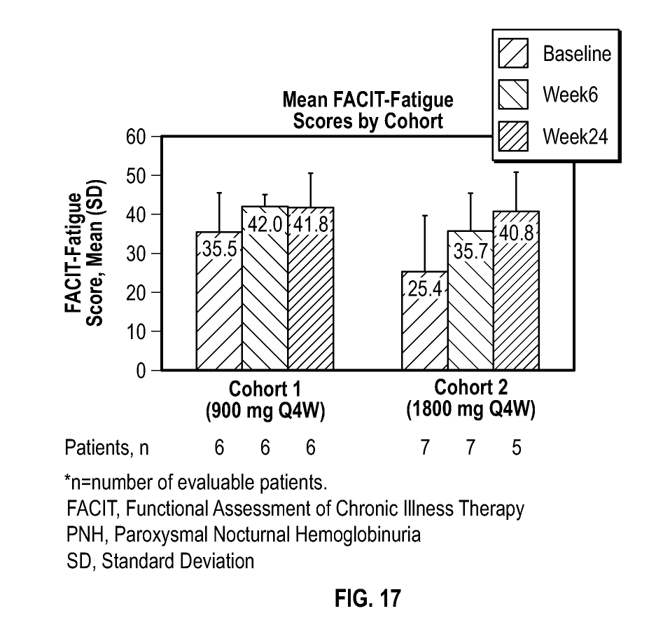

Median duration of exposure was 5.6 (5.5-5.6) months for C1 and 4.6 (3.7-4.7) months for C2. Patients had evidence of high hemolytic activity at baseline (BL), with LDH levels approximately 7-fold higher than the upper limit of normal (ULN). LDH levels decreased rapidly by the first evaluable time point (day 8), and improvements were sustained throughout all dosing intervals. Mean percentage reductions from BL in LDH levels were 85.9% in C1 at week 24 (n=6) and 85.2% in C2 at week 20 (n=5), the last available time points (FIG. 17). LDH levels were normalized in 4/6 patients (67%) in Cohort 1 and in 4/5 patients (80%) in Cohort 2 (FIG. 17). LDH levels ≤1.5×ULN were achieved by 5/6 patients (83%) in Cohort 1 and by all (5/5) patients (100%) in Cohort 2 (FIG. 17). Respective LDH mean (SD) values at these time points were 232 (82) and 198 (36) U/L for Cohorts 1 and 2, respectively. Mean hemoglobin (Hb) levels were improved or stable in both cohorts.

Among 5 patients with red blood cell transfusions in the 12 months prior to treatment (2 in C1, 3 in C2), only 1/2 pts (50%) in C1, became free of transfusions, while 3/3 patients (100%) in C2 were transfusion free (Table 17). The patient in C1 who required transfusions had LDH levels of 391 and 417 U/L (1.67 and 1.78×ULN), and Hb levels of 7.7 and 6.9 g/dL (BL Hb of 9.8 g/dL) on days 57 and 141, respectively. This patient consequently received 2 transfusions (2 units each) on these days. No patients in C2 required a transfusion.

TABLE 17
Transfusion Independence for Patients with History of
RBC Transfusion by Cohort

| | Cohort 1 | | Cohort 2 | |
|---|---|---|---|---|
| Parameter | (n = 6) | | (n = 7) | |
| Patients with transfusion within 1 year | 2 | (33) | 3 | (43) |
| prior to first ALXN1210 dose, n (%) | | | | |
| Patients with transfusion independence | 1/2 | (50%) | 3/3 | (100%) |
| since the first ALXN1210 dose^{a}, n (%) | | | | |

^{a}The median (range) follow-up time on ALXN1210 treatment was 5.6 (5.5-5.6) months in Cohort 1 and 4.6 (3.7-4.7) months in Cohort 2.

The values set forth in Table 18 are based on the original data from each patient and represent mean (SD) FACIT-Fatigue Scores over time. The mean score at baseline was lower in Cohort 2 compared with Cohort 1 (10.1 points), indicating more severe fatigue. Mean (SD) FACIT-Fatigue scores increased from BL to Week 6 (Day 43) by 28.9% (45.6%) in C1, and by 61.4% (49.9%) in C2 (mean of per-patient percentage changes). At Day 169 (week 24), FACIT-Fatigue scores were maintained in C1 (28.7% [52.7%] improvement from BL), but improved by 76.2% (70.3%) in C2 (mean of per-patient percentage changes). The latter values are based on mean (SD) percentage change from baseline in FACIT-Fatigue Scores (i.e., each patient's percentage change from baseline is calculated and an average is then calculated within each cohort). At week 24, mean (SD) scores were maintained in Cohort 1 (28.7% [52.7%] improvement from BL, n=6) but improved by 76.2% (70.3%) in Cohort 2 (n=5) (mean of per-patient percentage changes). It should be noted that the BL FACIT-Fatigue score was lower in C2 than in C1 (10.1 points), indicating more severe fatigue.

TABLE 18
FACIT-Fatigue Scores by Cohort*

| | Cohort 1 | Cohort 2 |
|---|---|---|
| FACIT-Fatigue Score | (n = 6) | (n = 7) |
| Baseline | | |
| n | 6 | 7 |
| Mean (SD) | 35.5 (10.03) | 25.4 (14.35) |
| Median (range) | 37.5 (17.0-45.0) | 19.0 (10.0-51.0) |
| Week 6 | | |
| n | 6 | 7 |
| Mean (SD) | 42.0 (3.03) | 35.7 (9.76) |
| Median (range) | 43.0 (37.0-45.0) | 37.0 (24.0-50.0) |
| Change from baseline | | |
| Mean (SD) | 6.5 (8.17) | 10.3 (6.42) |
| Median (range) | 4.5 (−3.0-20.0) | 12.0 (−1.0-18.0) |
| Percent change from baseline | | |
| Mean (SD) | 28.9 (45.6) | 61.4 (49.9) |
| Median (range) | 12.1 (−6.7-117.6) | 46.2 (−2.0-140.0) |
| Week 24 | | |
| n | 6 | 5 |
| Mean (SD) | 41.8 (8.77) | 40.8 (9.98) |
| Median (range) | 44.5 (26.0-50.0) | 43.0 (24.0-50.0) |
| Change from baseline | | |
| Mean (SD) | 6.3 (11.38) | 12.6 (9.76) |
| Median (range) | 6.0 (−12.0-21.0) | 14.0 (−1.0-25.0) |
| Percent change from baseline | | |
| Mean (SD) | 28.7 (52.70) | 76.2 (70.3) |
| Median (range) | 15.0 (−31.6-123.5) | 65.4 (−2.0-156.3) |

Note:
The FACIT-Fatigue is a 13-item scale evaluating the intensity of fatigue and impact on daily life using a 5-point Likert-type scale. The score ranges from 0 (maximum fatigue) to 52 (no fatigue) (see, e.g., Yellen SB, et al., J. Pain Symptom Manage. 1997; 13(2): 63-74, and Cella D, et al., Cancer. 2002; 94(2): 528-38). An increase in score of ≥3 on this instrument is considered clinically important (see Cella D, et al. J Pain Symptom Manage. 2002; 24: 547-561).
FACIT, Functional Assessment of Chronic Illness Therapy;
PNH, paroxysmal nocturnal hemoglobinuria;
SD, standard deviation.

PK analysis of available data revealed an estimated mean±SD terminal (beta) half-life of ALXN1210 of 42±6 days. No deaths, serious adverse events (AEs), drug discontinuations, or AEs leading to withdrawals were reported in either cohort. TEAEs during ALXN1210 treatment occurred in 5 patients (83.3%) in Cohort 1 and 6 patients (85.7%) in Cohort 2 (Table 19). The most common treatment-emergent AE (TEAE) was headache (4 patients; 30.8%). Multiple doses of ALXN1210 resulted in no serious AEs, withdrawals from the study, or deaths. Investigators judged 76.9% of TEAEs to be unrelated to treatment. All related TEAEs resolved during ongoing ALXN1210 treatment. All AEs considered at least possibly related to therapy over 4.6 to 5.6 months of median exposure, including atrial flutter, general ache, headache, and worsening of anemia, resolved with ongoing ALXN1210 treatment.

TABLE 19
Treatment-Emergent Adverse Events by Cohort

| | Cohort 1 | Cohort 2 | Overall |
|---|---|---|---|
| Parameter | (n = 6) | (n = 7) | (N = 13) |
| Patients with serious TEAEs, n (%) | 0 | 0 | 0 |
| Patients with TEAEs, n (%) | 5 (83) | 6 (86) | 11 (85) |
| Patients with related TEAEs^{a}, n (%) | 1 (17) | 2 (29) | 3 (23) |
| Anemia | 1 (17)^{b} | 0 | 1 (8) |
| Atrial flutter | 0 | 1 (14)^{c} | 1 (8) |
| Headache | 0 | 1 (14)^{d} | 1 (8) |
| Pain | 0 | 1 (14)^{e} | 1 (8) |

^{a}Judged by the investigator to be possibly related to ALXN1210 treatment.
^{b}Two events occurred on days 56 and 140 (both grade 3) and both resolved with transfusions.
^{c}Occurred on day 72 (grade 3) and resolved.
^{d}Five events occurred on days 1, 15, 29, 55, and 83 (all grade 2) and all resolved.
^{e}Occurred on day 57 (grade 1) and resolved.
TEAEs, treatment-emergent adverse events.

In sum, in patients with PNH previously-naïve to complement inhibitor therapy, ALXN1210 treatment resulted in rapid, complete, and sustained C5 inhibition with resultant reductions in LDH from baseline (˜85% in both cohorts). LDH levels were rapidly reduced in all patients. Mean LDH levels were reduced to below the ULN in 4/6 (67%) of patients in C1, and in 6/7 (86%) of patients in C2 at the last evaluable time point. A higher proportion of patients in the higher dose group achieved normal LDH levels (80% vs. 67%). A higher proportion of patients in the higher dose group achieved LDH<1.5×ULN (100% vs. 83%). At the last evaluable time point, LDH levels were reduced to below the ULN in 4/6 patients in Cohort 1 and 4/5 patients in Cohort 2. LDH levels <1.5×ULN were achieved in 5/6 and 5/5 patients, respectively. While there was a notable decrease in the need for blood transfusions, 17% (⅙) of patients in C1 (900 mg) experienced a recurrence of hemolysis and required transfusions, while no patients in C2 (1800 mg) had evidence of hemolysis or required a transfusion. A higher proportion of high dose patients became transfusion independent (100% or 3/3 vs. 50% or ½). Improvement in FACIT Fatigue was 2-fold greater with the higher dose cohort, consistent with better hemolytic response, and consistent with the clinical benefit demonstrated by eculizumab (Socié G, et al. Blood. 2007; 110:3672 and Muus P, et al. Haematologica. 2013; 98(s1) 83, P193). Compared with other measures, mean changes in LDH appears least sensitive to changes in dose, suggesting that this endpoint and this may not be the best suited to evaluate optimal response across dose groups. All other measures demonstrated numeric trends favoring the higher dose (1800 mg Q4Q) versus the lower dose (900 mg Q4Q) cohort. ALXN1210 treatment resulted in mean percentage reductions from baseline in LDH levels of 85.9% in Cohort 1 (n=6) and 85.2% in Cohort 2 (n=5). Compared with other measures, mean change in LDH appears least sensitive to changes in dose, suggesting that this endpoint may not be the best suited to evaluate optimal response across dose groups. Thus, the lower dose of 900 mg Q4W appeared inadequate in comparison to 1800 mg Q8W throughout the monthly dosing interval for complete suppression of complement-mediated hemolysis, which is central to the morbidities and mortality in PNH. These data suggest that the lower dose may be inadequate throughout the monthly dosing interval for complete suppression of hemolysis, which is central to the morbidities of PNH.

### SECTION 2: A PHASE 2, OPEN-LABEL, MULTIPLE ASCENDING DOSE STUDY IN PNH PATIENTS (EXAMPLES 6-8)

### Example 6: Overview of Study

An open-label, multiple ascending dose study is conducted to explore the efficacy, safety, tolerability, pharmacokinetics (PK), pharmacodynamics (PD), and immunogenicity of antibody BNJ441 (also known as ALXN1210) in patients with PNH who have not previously been treated with a complement inhibitor.

1. Objectives

The primary objective of the study is to evaluate the efficacy, safety, and tolerability, of multiple intravenous (IV) doses of ALXN1210 administered to complement inhibitor treatment-naïve patients with PNH.

Secondary objectives include characterizing the PK and PD effects of multiple IV doses of ALXN1210 administered to complement inhibitor treatment-naïve patients with PNH and investigating the immunogenicity of ALXN1210 administered IV to complement inhibitor treatment-naïve patients with PNH.

2. Study Design

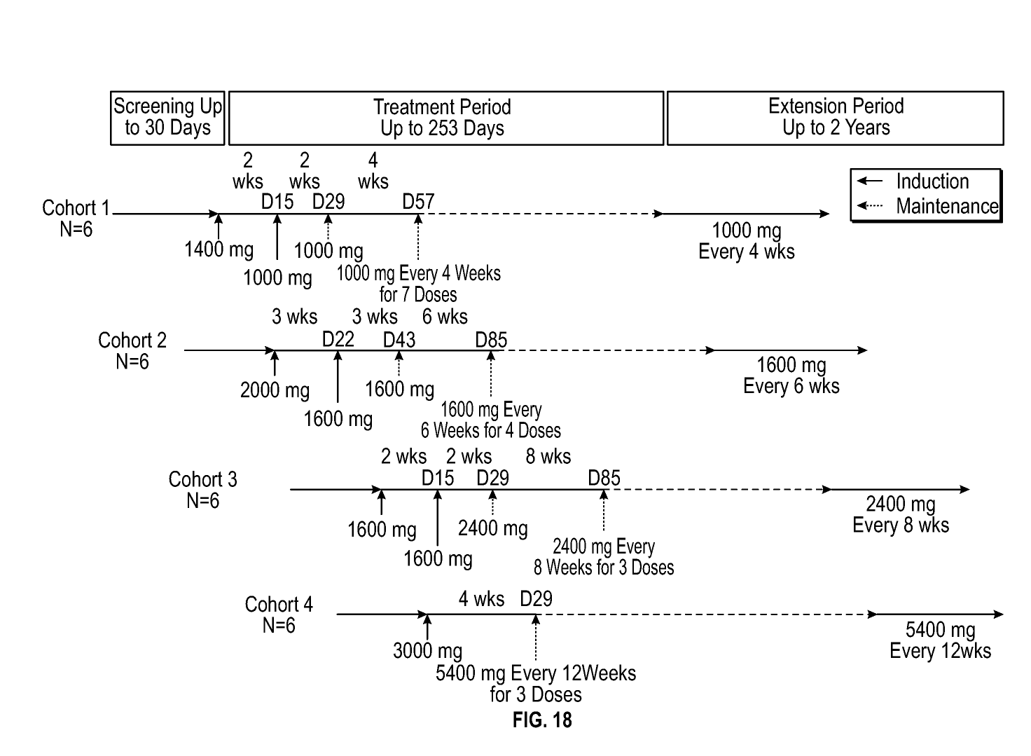

The overall study design, treatments and study duration is depicted in FIG. 18.

Four treatment cohorts and up to 26 patients (at least 6 per cohort) are enrolled, with at least 20 patients planned for evaluation. All patients are screened for study eligibility after providing written informed consent to participate. Patients who fail to meet any of the eligibility criteria are rescreened once for study participation, at the discretion of the Investigator.

Patients enrolled in Cohort 1 receive induction doses of ALXN1210 of 1400 mg on Day 1 and 1000 mg on Day 15. On Day 29, they receive the first of 8 maintenance doses of 1000 mg of ALXN1210 (administered every 28 days or 4 weeks).

Patients enrolled in Cohort 2 receive induction doses of ALXN1210 of 2000 mg on Day 1 and 1600 mg on Day 22. On Day 43, they receive the first of 5 maintenance doses of 1600 mg of ALXN1210 (administered every 42 days or 6 weeks).

Patients enrolled in Cohort 3 receive induction doses of ALXN1210 of 1600 mg on Day 1 and 1600 mg on Day 15. On Day 29, they receive the first of 4 maintenance doses of 2400 mg of ALXN1210 (administered every 56 days or 8 weeks).

Patients enrolled in Cohort 4 receive an induction dose of ALXN1210 of 3000 mg on Day 1. On Day 29, they receive the first of 3 maintenance doses of 5400 mg of ALXN1210 (administered every 84 days or 12 weeks). The first 2 patients in Cohort 4 receive their induction dose (3000 mg) at least 1 day apart. The third patient receives the induction dose at least 7 days after the second patient has received the induction dose.

The dosing schedule is provided in Table 1:

TABLE 1
Dosing Schedule

| Cohort | Patients | Induction | Maintenance | Extension |
|---|---|---|---|---|
| 1 | 6 | 1400 mg on | 1000 mg on Day 29 and then | Every 4 weeks |
| | | Day 1 | every 28 days or 4 weeks | Days 337, 505, 673, |
| | | 1000 mg on | (Days 57, 85, 113, 141, 169, | 841, 1009 |
| | | Day 15 | 197, 225) | |
| | | | 8 maintenance doses | |
| 2 | 6 | 2000 mg on | 1600 mg on Day 43 and then | Every 6 weeks |
| | | Day 1 | every 42 days or 6 weeks | Days 337, 505, 673, |
| | | 1600 mg on | (Days 85, 127, 169, 211) | 841, and 1009 |
| | | Day 22 | 5 maintenance doses | |
| 3 | 6 | 1600 mg on | 2400 mg on Day 29 and then | Every 8 weeks |
| | | Day 1 | every 56 days or 8 weeks | Days 309, 477, 645, |
| | | 1600 mg on | (Days 85, 141, 197) | 813, and 981 |
| | | Day 15 | 4 maintenance doses | |
| 4 | 6 to 8 | 3000 mg on | 5400 mg on Day 29 and | Every 12 weeks |
| | | Day 1 | then every 84 days or 12 | Days 281, 449, 617, |
| | | | weeks (Days 113 and 197) | 785, and 1037 |
| | | | 3 maintenance doses | |

An independent Data Monitoring Committee (DMC) reviews and evaluates the study data for patient safety and makes recommendations on dose escalation, continuing dosing within the cohort, modification, or termination of the study.

The DMC conducts a review of the available safety data at the following scheduled time points:

Fifteen days after the second patient in Cohort 1 receives the second induction dose to determine whether Cohort 2 is opened.

Fifteen days after the second patient in Cohort 2 receives the second induction dose to determine whether Cohort 3 is opened.

Fifteen days after the second patient in Cohort 3 receives the first maintenance dose to determine whether Cohort 4 is opened.

Approximately 7 days after the second patient in Cohort 4 receives the 3000 mg dose to review safety.

Approximately 7 days after the second patient in Cohort 4 receives the first 5400 mg dose to review safety.

If additional patients are screened and eligible for enrollment before a dose-escalation decision is made by the DMC for any cohort, those patients are assigned to the active cohort with the lowest dose level.

On Day 253, patients continue treatment in a long-term Extension Period of the study, at the same maintenance dose and frequency as their final dose of ALXN1210 administered during the Treatment Period.

Up to 30 days will be allowed for screening procedures. The total duration of treatment (which includes an Induction Period and a Maintenance Period) is approximately 253 days. The total duration of the Extension Period is up to 2 years.

All patients are monitored closely for signs of infection throughout the study. Treatment with prophylactic antibiotics is at the discretion of the Investigator and per the site/country standard of care.

3. Dose Rationale

Initial analyses supporting dose and dose regimen selection for this study utilized data from the single-ascending dose (SAD) study in healthy volunteers. The PK data following single sub-therapeutic doses (200 mg and 400 mg) of ALXN1210 to healthy volunteers (ALXN1210-HV-101) yielded an estimated mean (range) elimination half-life of ALXN1210 of −31 (27-38) days. The initial PK/PD data estimated a target efficacious trough level of ≥100 μg/mL to design dose regimens for this study. The targeted efficacious trough level is defined as concentration achieving complete complement inhibition (≥99%) in all patients (≥97.5%). Subsequent population PK analyses, utilizing emerging PK data from both healthy volunteers and patients with PNH across a wider range of doses, exposures, and durations, suggest the median (95% confidence interval) elimination half-life of ALXN1210 is −43 days (39-48). This change in the estimate of elimination half-life of ALXN1210 following multiple dose administrations in healthy volunteers and patients with PNH has resulted in an increase in the estimated target trough concentrations in Cohorts 1-3 and has presented the opportunity to explore dosing ALXN1210 less frequently. It is also desirable to target a trough level that achieves complete complement inhibition in all PNH patients. Therefore, to provide safety and PK/PD support for an optimized Phase 3 dosing regimen characterization, it is necessary to evaluate the dose regimen ranging in this protocol from once every month dosing to once every 3 month dosing as enabled by the increased estimated elimination half-life, taking into account the current estimates of targeted efficacious ALXN1210 trough level.

4. Schedule of Assessments

The Schedule of Assessments is set forth in Tables 2-9:

TABLE 2
Schedule of Assessments: Induction and Maintenance Dosing During the Treatment Period: Cohort 1

| | | Treatment | | | | | | | | | | | | | | | |
|---|---|---|---|---|---|---|---|---|---|---|---|---|---|---|---|---|---|
| Period | Screening | Induction | | | | Maintenance | | | | | | | | | | | |
| Study Day | −30 to −1 | 1 | 7 | 15 | 22 | 29 | 43 | 57 | 85 | 113 | 127 | 141 | 169 | 197 | 211 | 225 | 253¹ |
| Window (day) | N/A | ±1 | ±1 | ±1 | ±1 | ±1 | ±1 | ±1 | ±2 | ±2 | ±2 | ±2 | ±2 | ±2 | ±2 | ±2 | ±2 |
| Informed | X | | | | | | | | | | | | | | | | |
| consent | | | | | | | | | | | | | | | | | |
| Confirmation of | X | | | | | | | | | | | | | | | | |
| meningococcal | | | | | | | | | | | | | | | | | |
| vaccination² | | | | | | | | | | | | | | | | | |
| Medical history | X | | | | | | | | | | | | | | | | |
| and | | | | | | | | | | | | | | | | | |
| demographics | | | | | | | | | | | | | | | | | |
| Virus serology³ | X | | | | | | | | | | | | | | | | |
| PNH clone size⁴ | X | X | | | | | | X | | X | | | X | | | X | X |
| Height, weight, | X | X | | | | | | | | | | | | | | | |
| BMI⁵ | | | | | | | | | | | | | | | | | |
| Pregnancy test⁶ | X | X | | X | | X | | X | X | X | | X | X | X | | X | X |
| PNH | X | X | X | X | X | X | X | X | X | X | X | X | X | X | X | X | X |
| symptomatology⁷ | | | | | | | | | | | | | | | | | |
| Physical | X | | | | | | | | | | | | | | | | X |
| examination | | | | | | | | | | | | | | | | | |
| Abbreviated | | X | | X | | X | | X | X | X | | X | X | X | | X | |
| physical exam⁸ | | | | | | | | | | | | | | | | | |
| Vital signs | X | X | X | X | X | X | X | X | X | X | X | X | X | X | X | X | X |
| 12-Lead ECG⁹ | X | X | | | | | | | | | | | | | | | X |
| Chemistry, | X¹¹ | X | X | X | X | X | X | X | X | X | X | X | X | X | X | X | X |
| including LDH¹⁰ | | | | | | | | | | | | | | | | | |
| Hematology¹² | X | X | X | X | X | X | X | X | X | X | X | X | X | X | X | X | X |
| Coagulation | X | X | X | X | X | X | X | X | X | X | X | X | X | X | X | X | X |
| Urinalysis and | X | X | | | | | | | X | | | | X | | | | X |
| urine chemistry, | | | | | | | | | | | | | | | | | |
| spot urine | | | | | | | | | | | | | | | | | |
| eGFR | X | X | | | | | | | X | | | | X | | | | X |
| calculation | | | | | | | | | | | | | | | | | |
| Brain natriuretic | | X | | | | X | | | X | | | X | | X | | | X |
| peptide¹³ | | | | | | | | | | | | | | | | | |
| ALXN1210 | | X | | X | | X | | X | X | X | | X | X | X | | X | X |
| administration | | | | | | | | | | | | | | | | | |
| PK sampling¹⁴ | | X | | X | X | X | X | X | X | X | X | X | X | X | X | X | X |
| Serum PD | | X | | X | X | X | X | X | X | X | X | X | X | X | X | X | X |
| panel¹⁵ | | | | | | | | | | | | | | | | | |
| Immunogenicity | | X | | X | | X | | X | X | X | | X | X | X | | X | X |
| (ADA)¹⁶ | | | | | | | | | | | | | | | | | |
| QoL | | X | | X | | | | X | | X | X | | | X | | X | X |
| assessments¹⁷ | | | | | | | | | | | | | | | | | |
| Infusion site | | X | | X | | X | | X | X | X | | X | X | X | | X | X |
| evaluation¹⁸ | | | | | | | | | | | | | | | | | |
| VAS for | | X | | X | | X | | X | X | X | | X | X | X | | X | X |
| infusion site | | | | | | | | | | | | | | | | | |
| pain¹⁹ | | | | | | | | | | | | | | | | | |

| | |
|---|---|
| Review safety | ← ← ← Review continuously → → → |
| card | |
| Concomitant | ← ← ← Monitor continuously → → → |
| medications | |
| Adverse events | ← ← ← Monitor continuously → → → |

Abbreviations: ADA = antidrug antibody; BMI = body mass index; ECG = electrocardiogram; eGFR = estimated glomerular filtration rate; LDH = lactate dehydrogenase; QoL = quality of life; PD = pharmacodynamic; PK = pharmacokinetic; PNH = paroxysmal nocturnal hemoglobinuria; VAS = visual analog scale
¹All assessments are performed predose and are part of the Maintenance Period. The dose administered on Day 253 is the first dose in the Extension Period.
²Meningococcal vaccination can be completed on Day 1 prior to dosing with ALXN1210. Prophylactic antibiotics must be used if 14 days has not elapsed.
³Hepatitis B and C, human immunodeficiency virus types 1 and 2.
⁴Granulocyte and red blood cell clone size at screening and red blood cell clone size only during the Treatment Period.
⁵Measure height at screening only.
⁶Female patients of childbearing potential only. Serum pregnancy test at screening only; urine pregnancy test at all other time points. A negative urine test result is required prior to administering ALXN1210 to female patients on dosing days.
⁷Investigator assessment of the following events: fatigue, abdominal pain, dyspnea, dysphagia, chest pain, and erectile dysfunction. Symptoms of disease burden are captured through the QoL questionnaires.
⁸Abbreviated physical examination consists of a body system relevant examination based upon Investigator judgment and subject symptoms.
⁹Obtain triplicate 12-lead ECGs at screening and prior to the first dose on Day 1, and single 12-lead ECGs predose on Day 253.
¹⁰Clinical safety laboratory measurements are collected predose on dosing days. Follicle-stimulating hormones and estradiol levels are measured at least twice during screening, only in order to confirm postmenopausal status.
¹¹At least two samples re collected at least one day apart during the screening period for LDH testing.
¹²Assessment for safety, as well as the following parameters as secondary endpoints: free hemoglobin, haptoglobin, reticulocyte count, and D-dimer.
¹³Obtain predose on Days 1, 29, 85, 141, 197, and 253.
¹⁴Cohort-specific sampling time points.
¹⁵Serum for exploratory PD assays; cohort-specific sampling time points.
¹⁶Immunogenicity samples are collected predose on dosing days.
¹⁷Functional Assessment of Chronic Illness Therapy-Fatigue Scale, version 4.0 and European Organisation for Research and Treatment of Cancer, Quality of Life Questionnaire-Core 30 Scale, Version 3.0.
¹⁸An induration or reaction of < 10 mm is not be listed as an adverse event unless it persists for more than 24 hours, at which time the patient must inform the study staff immediately and proceed to the nearest hospital emergency department.
¹⁹Patient assess infusion site pain using a 100 mm VAS. The VAS is completed as soon as practical after completion of the infusion.

TABLE 3
Schedule of Assessments: Induction and Maintenance Dosing During the Treatment Period: Cohort 2

| | | Treatment | | | | | | | | | | | | | | | |
|---|---|---|---|---|---|---|---|---|---|---|---|---|---|---|---|---|---|
| Period | Screening | Induction | | | | Maintenance | | | | | | | | | | | |
| Study Day | −30 to −1 | 1 | 7 | 15 | 22 | 29 | 43 | 57 | 85 | 113 | 127 | 141 | 169 | 197 | 211 | 225 | 253¹ |
| Window (day) | N/A | ±1 | ±1 | ±1 | ±1 | ±1 | ±1 | ±1 | ±2 | ±2 | ±2 | ±2 | ±2 | ±2 | ±2 | ±2 | ±2 |
| Informed | X | | | | | | | | | | | | | | | | |
| consent | | | | | | | | | | | | | | | | | |
| Confirmation of | X | | | | | | | | | | | | | | | | |
| meningococcal | | | | | | | | | | | | | | | | | |
| vaccination² | | | | | | | | | | | | | | | | | |
| Medical history | X | | | | | | | | | | | | | | | | |
| and | | | | | | | | | | | | | | | | | |
| demographics | | | | | | | | | | | | | | | | | |
| Virus serology³ | X | | | | | | | | | | | | | | | | |
| PNH clone size⁴ | X | X | | | | | | X | | X | | | X | | | X | X |
| Height, weight, | X | X | | | | | | | | | | | | | | | |
| BMI⁵ | | | | | | | | | | | | | | | | | |
| Pregnancy test⁶ | X | X | | | X | | X | | X | | X | | X | | X | | X |
| PNH | X | X | X | X | X | X | X | X | X | X | X | X | X | X | X | X | X |
| symptomatology⁷ | | | | | | | | | | | | | | | | | |
| Physical | X | | | | | | | | | | | | | | | | X |
| examination | | | | | | | | | | | | | | | | | |
| Abbreviated | | X | | | X | | X | | X | | X | | X | | X | | |
| physical exam⁸ | | | | | | | | | | | | | | | | | |
| Vital signs | X | X | X | X | X | X | X | X | X | X | X | X | X | X | X | X | X |
| 12-Lead ECG⁹ | X | X | | | | | | | | | | | | | | | X |
| Chemistry, | X¹¹ | X | X | X | X | X | X | X | X | X | X | X | X | X | X | X | X |
| including LDH¹⁰ | | | | | | | | | | | | | | | | | |
| Hematology¹² | X | X | X | X | X | X | X | X | X | X | X | X | X | X | X | X | X |
| Coagulation | X | X | X | X | X | X | X | X | X | X | X | X | X | X | X | X | X |
| Urinalysis and | X | X | | | | | | | X | | | | X | | | | X |
| urine chemistry, | | | | | | | | | | | | | | | | | |
| spot urine | | | | | | | | | | | | | | | | | |
| eGFR | X | X | | | | | | | X | | | | X | | | | X |
| calculation | | | | | | | | | | | | | | | | | |
| Brain natriuretic | | X | | | X | | | | X | | X | | X | | | | X |
| peptide¹³ | | | | | | | | | | | | | | | | | |
| ALXN1210 | | X | | | X | | X | | X | | X | | X | | X | | X |
| administration | | | | | | | | | | | | | | | | | |
| PK sampling¹⁴ | | X | | | X | X | X | X | X | X | X | X | X | X | X | X | X |
| Serum PD panel¹⁵ | | X | | | X | X | X | X | X | X | X | X | X | X | X | X | X |
| Immunogenicity | | X | | | X | | X | | X | | X | | X | | X | | X |
| (ADA)¹⁶ | | | | | | | | | | | | | | | | | |
| QoL | | X | | X | | | | X | | X | X | | | X | | X | X |
| assessments¹⁷ | | | | | | | | | | | | | | | | | |
| Infusion site | | X | | | X | | X | | X | | X | | X | | X | | X |
| evaluation¹⁸ | | | | | | | | | | | | | | | | | |
| VAS for | | X | | | X | | X | | X | | X | | X | | X | | X |
| infusion site | | | | | | | | | | | | | | | | | |
| pain¹⁹ | | | | | | | | | | | | | | | | | |

| | |
|---|---|
| Review safety | ← ← ← Review continuously → → → |
| card | |
| Concomitant | ← ← ← Monitor continuously → → → |
| medications | |
| Adverse events | ← ← ← Monitor continuously → → → |

Abbreviations: ADA = antidrug antibody; BMI = body mass index; ECG = electrocardiogram; eGFR = estimated glomerular filtration rate; LDH = lactate dehydrogenase; QoL = quality of life; PD = pharmacodynamic; PK = pharmacokinetic; PNH = paroxysmal nocturnal hemoglobinuria; VAS = visual analog scale
¹All assessments are performed predose and are part of the Maintenance Period. The dose administered on Day 253 is the first dose in the Extension Period.
²Meningococcal vaccination can be completed on Day 1 prior to dosing with ALXN1210. Prophylactic antibiotics must be used if 14 days has not elapsed.
³Hepatitis B and C, human immunodeficiency virus types 1 and 2.
⁴Granulocyte and red blood cell clone size at screening and red blood cell clone size only during the Treatment Period.
⁵Measure height at screening only.
⁶Female patients of childbearing potential only. Serum pregnancy test at screening only; urine pregnancy test at all other time points. A negative urine test result is required prior to administering ALXN1210 to female patients on dosing days.
⁷Investigator assessment of the following events: fatigue, abdominal pain, dyspnea, dysphagia, chest pain, and erectile dysfunction. Symptoms of disease burden will be captured through the QoL questionnaires.
⁸Abbreviated physical examination consists of a body system relevant examination based upon Investigator judgment and subject symptoms.
⁹Obtain triplicate 12-lead ECGs at screening and prior to the first dose on Day 1, and single 12-lead ECGs predose on Day 253.
¹⁰Clinical safety laboratory measurements are collected predose on dosing days. Follicle-stimulating hormones and estradiol levels are measured at least twice during screening, only in order to confirm postmenopausal status.
¹¹At least two samples must be collected at least one day apart during the screening period for LDH testing.
¹²Assessment for safety, as well as the following parameters as secondary endpoints: free hemoglobin, haptoglobin, reticulocyte count, and D-dimer.
¹³Obtain predose on Days 1, 22, 85, 127, 169, and 253.
¹⁴Cohort-specific sampling time points.
¹⁵Serum for exploratory PD assays; cohort-specific sampling time points.
¹⁶Immunogenicity samples are collected predose on dosing days.
¹⁷Functional Assessment of Chronic Illness Therapy-Fatigue Scale, version 4.0 and European Organisation for Research and Treatment of Cancer, Quality of Life Questionnaire-Core 30 Scale, Version 3.0.
¹⁸An induration or reaction of < 10 mm is be listed as an adverse event unless it persists for more than 24 hours, at which time the patient must inform the study staff immediately and proceed to the nearest hospital emergency department.
¹⁹Patient assess infusion site pain using a 100 mm VAS. The VAS is completed as soon as practical after completion of the infusion.

TABLE 4
Schedule of Assessments: Induction and Maintenance Dosing During the Treatment Period: Cohort 3

| | | Treatment | | | | | | | | | | | | | | | |
|---|---|---|---|---|---|---|---|---|---|---|---|---|---|---|---|---|---|
| Period | Screening | Induction | | | | Maintenance | | | | | | | | | | | |
| Study Day | −30 to −1 | 1 | 7 | 15 | 22 | 29 | 43 | 57 | 85 | 113 | 127 | 141 | 169 | 197 | 211 | 225 | 253¹ |
| Window (day) | N/A | ±1 | ±1 | ±1 | ±1 | ±1 | ±1 | ±1 | ±2 | ±2 | ±2 | ±2 | ±2 | ±2 | ±2 | ±2 | ±2 |
| Informed | X | | | | | | | | | | | | | | | | |
| consent | | | | | | | | | | | | | | | | | |
| Confirmation of | X | | | | | | | | | | | | | | | | |
| meningococcal | | | | | | | | | | | | | | | | | |
| vaccination² | | | | | | | | | | | | | | | | | |
| Medical history | X | | | | | | | | | | | | | | | | |
| and | | | | | | | | | | | | | | | | | |
| demographics | | | | | | | | | | | | | | | | | |
| Virus serology³ | X | | | | | | | | | | | | | | | | |
| PNH clone size⁴ | X | X | | | | | | X | | X | | | X | | | X | X |
| Height, weight, | X | X | | | | | | | | | | | | | | | |
| BMI⁵ | | | | | | | | | | | | | | | | | |
| Pregnancy test⁶ | X | X | | X | | X | | | X | | | X | | X | | | X |
| PNH | X | X | X | X | X | X | X | X | X | X | X | X | X | X | X | | X |
| symptomatology⁷ | | | | | | | | | | | | | | | | | |
| Physical | X | | | | | | | | | | | | | | | | X |
| examination | | | | | | | | | | | | | | | | | |
| Abbreviated | | X | | X | | X | | | X | | | X | | X | | | |
| physical exam⁸ | | | | | | | | | | | | | | | | | |
| Vital signs | X | X | X | X | X | X | X | X | X | X | X | X | X | X | X | X | X |
| 12-Lead ECG⁹ | X | X | | | | | | | | | | | | | | | X |
| Chemistry, | X¹¹ | X | X | X | X | X | X | X | X | X | X | X | X | X | X | X | X |
| including LDH¹⁰ | | | | | | | | | | | | | | | | | |
| Hematology¹² | X | X | X | X | X | X | X | X | X | X | X | X | X | X | X | X | X |
| Coagulation | X | X | X | X | X | X | X | X | X | X | X | X | X | X | X | X | X |
| Urinalysis and | X | X | | | | | | | X | | | | X | | | | X |
| urine chemistry, | | | | | | | | | | | | | | | | | |
| spot urine | | | | | | | | | | | | | | | | | |
| eGFR | X | X | | | | | | | X | | | | X | | | | X |
| calculation | | | | | | | | | | | | | | | | | |
| Brain natriuretic | | X | | | | X | | | X | | | X | | X | | | X |
| peptide¹³ | | | | | | | | | | | | | | | | | |
| ALXN1210 | | X | | X | | X | | | X | | | X | | X | | | X |
| administration | | | | | | | | | | | | | | | | | |
| PK sampling¹⁴ | | X | | X | X | X | X | X | X | X | X | X | X | X | X | X | X |
| Serum PD | | X | | X | X | X | X | X | X | X | X | X | X | X | X | X | X |
| panel¹⁵ | | | | | | | | | | | | | | | | | |
| Immunogenicity | | X | | X | | X | | | X | | | X | | X | | | X |
| (ADA)¹⁶ | | | | | | | | | | | | | | | | | |
| QoL | | X | | X | | | | X | | X | X | | | X | | X | X |
| assessments¹⁷ | | | | | | | | | | | | | | | | | |
| Infusion site | | X | | X | | X | | | X | | | X | | X | | | X |
| evaluation¹⁸ | | | | | | | | | | | | | | | | | |
| VAS for | | X | | X | | X | | | X | | | X | | X | | | X |
| infusion site | | | | | | | | | | | | | | | | | |
| pain¹⁹ | | | | | | | | | | | | | | | | | |

| | |
|---|---|
| Review safety | ← ← ← Review continuously → → → |
| card | |
| Concomitant | ← ← ← Monitor continuously → → → |
| medications | |
| Adverse events | ← ← ← Monitor continuously → → → |

Abbreviations: ADA = antidrug antibody; BMI = body mass index; ECG = electrocardiogram; eGFR = estimated glomerular filtration rate; LDH = lactate dehydrogenase; QoL = quality of life; PD = pharmacodynamic; PK = pharmacokinetic; PNH = paroxysmal nocturnal hemoglobinuria; VAS = visual analog scale
¹All assessments are performed predose and are part of the Maintenance Period. The dose administered on Day 253 is the first dose in the Extension Period.
²Meningococcal vaccination can be completed on Day 1 prior to dosing with ALXN1210. Prophylactic antibiotics must be used if 14 days has not elapsed.
³Hepatitis B and C, human immunodeficiency virus types 1 and 2.
⁴Granulocyte and red blood cell clone size at screening and red blood cell clone size only during the Treatment Period.
⁵Measure height at screening only.
⁶Female patients of childbearing potential only. Serum pregnancy test at screening only; urine pregnancy test at all other time points. A negative urine test result is required prior to administering ALXN1210 to female patients on dosing days.
⁷Investigator assessment of the following events: fatigue, abdominal pain, dyspnea, dysphagia, chest pain, and erectile dysfunction. Symptoms of disease burden will be captured through the QoL questionnaires.
⁸Abbreviated physical examination consists of a body system relevant examination based upon Investigator judgment and subject symptoms.
⁹Obtain triplicate 12-lead ECGs at screening and prior to the first dose on Day 1, and single 12-lead ECGs predose on Day 253.
¹⁰Clinical safety laboratory measurements are collected predose on dosing days. Follicle-stimulating hormones and estradiol levels are measured at least twice during screening, only in order to confirm postmenopausal status.
¹¹At least two samples must be collected at least one day apart during the screening period for LDH testing.
¹²Assessment for safety, as well as the following parameters as secondary endpoints: free hemoglobin, haptoglobin, reticulocyte count, and D-dimer.
¹³Obtain predose on Days 1, 22, 85, 127, 169, and 253.
¹⁴Cohort-specific sampling time points.
¹⁵Serum for exploratory PD assays; cohort-specific sampling time points.
¹⁶Immunogenicity samples are collected predose on dosing days.
¹⁷Functional Assessment of Chronic Illness Therapy-Fatigue Scale, version 4.0 and European Organisation for Research and Treatment of Cancer, Quality of Life Questionnaire-Core 30 Scale, Version 3.0.
¹⁸An induration or reaction of < 10 mm is be listed as an adverse event unless it persists for more than 24 hours, at which time the patient must inform the study staff immediately and proceed to the nearest hospital emergency department.
¹⁹Patient assess infusion site pain using a 100 mm VAS. The VAS is completed as soon as practical after completion of the infusion.

TABLE 5
Schedule of Assessments: Induction and Maintenance Dosing During the Treatment Period: Cohort 4

| | | Treatment | | | | | | | | | | | | | | | |
|---|---|---|---|---|---|---|---|---|---|---|---|---|---|---|---|---|---|
| Period | Screening | Induction | | | | Maintenance | | | | | | | | | | | |
| Study Day | −30 to −1 | 1 | 7 | 15 | 22 | 29 | 43 | 57 | 85 | 113 | 127 | 141 | 169 | 197 | 211 | 225 | 253¹ |
| Window (day) | N/A | ±1 | ±1 | ±1 | ±1 | ±1 | ±1 | ±1 | ±2 | ±2 | ±2 | ±2 | ±2 | ±2 | ±2 | ±2 | ±2 |
| Informed | X | | | | | | | | | | | | | | | | |
| consent | | | | | | | | | | | | | | | | | |
| Confirmation of | X | | | | | | | | | | | | | | | | |
| meningococcal | | | | | | | | | | | | | | | | | |
| vaccination² | | | | | | | | | | | | | | | | | |
| Medical history | X | | | | | | | | | | | | | | | | |
| and | | | | | | | | | | | | | | | | | |
| demographics | | | | | | | | | | | | | | | | | |
| Virus serology³ | X | | | | | | | | | | | | | | | | |
| PNH clone size⁴ | X | X | | | | | | X | | X | | | X | | | X | X |
| Height, weight, | X | X | | | | | | | | | | | | | | | |
| BMI⁵ | | | | | | | | | | | | | | | | | |
| Pregnancy test⁶ | X | X | | | | X | | | | X | | | | X | | | |
| PNH | X | X | X | X | X | X | X | X | X | X | X | X | X | X | X | X | X |
| symptomatology⁷ | | | | | | | | | | | | | | | | | |
| Physical | X | | | | | | | | | | | | | | | | X |
| examination | | | | | | | | | | | | | | | | | |
| Abbreviated | | X | | X | | X | | X | | X | | X | | X | | X | |
| physical exam⁸ | | | | | | | | | | | | | | | | | |
| Vital signs | X | X | X | X | X | X | X | X | X | X | X | X | X | X | X | X | X |
| 12-Lead ECG⁹ | X | X | | | | | | | | | | | | | | | X |
| Chemistry, | X¹¹ | X | X | X | X | X | X | X | X | X | X | X | X | X | X | X | X |
| including LDH¹⁰ | | | | | | | | | | | | | | | | | |
| Hematology¹² | X | X | X | X | X | X | X | X | X | X | X | X | X | X | X | X | X |
| Coagulation | X | X | X | X | X | X | X | X | X | X | X | X | X | X | X | X | X |
| Urinalysis and | X | X | | | | | | | X | | | | X | | | | X |
| urine chemistry, | | | | | | | | | | | | | | | | | |
| spot urine | | | | | | | | | | | | | | | | | |
| eGFR | X | X | | | | | | | X | | | | X | | | | X |
| calculation | | | | | | | | | | | | | | | | | |
| Brain natriuretic | | X | | | | X | | | | X | | | | X | | | X |
| peptide¹³ | | | | | | | | | | | | | | | | | |
| ALXN1210 | | X | | | | X | | | | X | | | | X | | | |
| administration | | | | | | | | | | | | | | | | | |
| PK sampling¹⁴ | | X | | X | | X | | X | | X | | X | | X | | X | X |
| Serum PD | | X | | X | | X | | X | | X | | X | | X | | X | X |
| panel¹⁵ | | | | | | | | | | | | | | | | | |
| Immunogenicity | | X | | | | X | | | | X | | | | X | | | |
| (ADA)¹⁶ | | | | | | | | | | | | | | | | | |
| QoL | | X | | X | | | | X | | X | X | | | X | | X | X |
| assessments¹⁷ | | | | | | | | | | | | | | | | | |
| Infusion site | | X | | | | X | | | | X | | | | X | | | |
| evaluation¹⁸ | | | | | | | | | | | | | | | | | |
| VAS for | | X | | | | X | | | | X | | | | X | | | |
| infusion site | | | | | | | | | | | | | | | | | |
| pain¹⁹ | | | | | | | | | | | | | | | | | |

| | |
|---|---|
| Review safety | ← ← ← Monitor continuously → → → |
| card | |
| Concomitant | ← ← ← Monitor continuously → → → |
| medications | |
| Adverse events | ← ← ← Monitor continuously → → → |

Abbreviations: ADA = antidrug antibody; BMI = body mass index; ECG = electrocardiogram; eGFR = estimated glomerular filtration rate; LDH = lactate dehydrogenase; QoL = quality of life; PD = pharmacodynamic; PK = pharmacokinetic; PNH = paroxysmal nocturnal hemoglobinuria; VAS = visual analog scale
¹All assessments are performed predose and are part of the Maintenance Period. The dose administered on Day 253 is the first dose in the Extension Period.
²Meningococcal vaccination can be completed on Day 1 prior to dosing with ALXN1210. Prophylactic antibiotics must be used if 14 days has not elapsed.
³Hepatitis B and C, human immunodeficiency virus types 1 and 2.
⁴Granulocyte and red blood cell clone size at screening and red blood cell clone size only during the Treatment Period.
⁵Measure height at screening only.
⁶Female patients of childbearing potential only. Serum pregnancy test at screening only; urine pregnancy test at all other time points. A negative urine test result is required prior to administering ALXN1210 to female patients on dosing days.
⁷Investigator assessment of the following events: fatigue, abdominal pain, dyspnea, dysphagia, chest pain, and erectile dysfunction. Symptoms of disease burden will be captured through the QoL questionnaires.
⁸Abbreviated physical examination consists of a body system relevant examination based upon Investigator judgment and subject symptoms.
⁹Obtain triplicate 12-lead ECGs at screening and prior to the first dose on Day 1, and single 12-lead ECGs predose on Day 253.
¹⁰Clinical safety laboratory measurements are collected predose on dosing days. Follicle-stimulating hormones and estradiol levels are measured at least twice during screening, only in order to confirm postmenopausal status.
¹¹At least two samples must be collected at least one day apart during the screening period for LDH testing.
¹²Assessment for safety, as well as the following parameters as secondary endpoints: free hemoglobin, haptoglobin, reticulocyte count, and D-dimer.
¹³Obtain predose on Days 1, 22, 85, 127, 169, and 253.
¹⁴Cohort-specific sampling time points.
¹⁵Serum for exploratory PD assays; cohort-specific sampling time points.
¹⁶Immunogenicity samples are collected predose on dosing days.
¹⁷Functional Assessment of Chronic Illness Therapy-Fatigue Scale, version 4.0 and European Organisation for Research and Treatment of Cancer, Quality of Life Questionnaire-Core 30 Scale, Version 3.0.
¹⁸An induration or reaction of < 10 mm is be listed as an adverse event unless it persists for more than 24 hours, at which time the patient must inform the study staff immediately and proceed to the nearest hospital emergency department.
¹⁹Patient assess infusion site pain using a 100 mm VAS. The VAS is completed as soon as practical after completion of the infusion.

TABLE 6
Schedule of Assessments: Extension Period and Early Termination: Cohort 1

| | Extension | | | | | | | | | | | | | | |
|---|---|---|---|---|---|---|---|---|---|---|---|---|---|---|---|
| Period | Year 1 | | | | Year 2 | | | | | | | | | | |
| Study Day | 281 | 309 | 337 | 365 | 393 | 421 | 449 | 447 | 505 | 533 | 561 | 589 | 617 | 645 | 673 |
| Window (day) | ±5 | ±5 | ±5 | ±5 | ±5 | ±5 | ±5 | ±5 | ±5 | ±5 | ±5 | ±5 | ±5 | ±5 | ±5 |
| ALXN1210 | X | X | X | X | X | X | X | X | X | X | X | X | X | X | X |
| administration | | | | | | | | | | | | | | | |
| PNH clone | | | X | | | | | | X | | | | | | X |
| size² | | | | | | | | | | | | | | | |
| Pregnancy test³ | X | X | X | X | X | X | X | X | X | X | X | X | X | X | X |
| PNH | X | X | X | X | X | X | X | X | X | X | X | X | X | X | X |
| symptomatology⁴ | | | | | | | | | | | | | | | |
| Physical | | | | | | | | | | | | | | | |
| examination | | | | | | | | | | | | | | | |
| Abbreviated | X | X | X | X | X | X | X | X | X | X | X | X | X | X | X |
| physical | | | | | | | | | | | | | | | |
| examination⁵ | | | | | | | | | | | | | | | |
| Vital signs | X | X | X | X | X | X | X | X | X | X | X | X | X | X | X |
| 12-Lead ECG⁶ | | | | | | | | | | | | | | | |
| Chemistry, | X | X | X | X | X | X | X | X | X | X | X | X | X | X | X |
| including LDH | | | | | | | | | | | | | | | |
| Hematology⁷ | X | X | X | X | X | X | X | X | X | X | X | X | X | X | X |
| Coagulation | X | X | X | X | X | X | X | X | X | X | X | X | X | X | X |
| Urinalysis and | X | X | X | X | X | X | X | X | X | X | X | X | X | X | X |
| urine chemistry | | | | | | | | | | | | | | | |
| PK sampling⁸ | | | X | | | | | | X | | | | | | X |
| Serum PD | | | X | | | | | | X | | | | | | X |
| panel⁹ | | | | | | | | | | | | | | | |
| Immunogenicity | | | X | | | | | | X | | | | | | X |
| (ADA)¹⁰ | | | | | | | | | | | | | | | |
| QoL | | | X | | | X | | | X | | | X | | | X |
| assessments | | | | | | | | | | | | | | | |
| Infusion site | X | X | X | X | X | X | X | X | X | X | X | X | X | X | X |
| evaluation¹¹ | | | | | | | | | | | | | | | |
| VAS for | X | X | X | X | X | X | X | X | X | X | X | X | X | X | X |
| infusion site | | | | | | | | | | | | | | | |
| pain¹² | | | | | | | | | | | | | | | |

| | |
|---|---|
| Review safety | ← ← ← Review continuously → → → |
| card | |
| Concomitant | ← ← ← Monitor continuously → → → |
| medications | |
| Adverse events | ← ← ← Monitor continuously → → → |

| | Extension | | | | | | | | | | | | |
|---|---|---|---|---|---|---|---|---|---|---|---|---|---|
| Period | Year 2 | | Year 3 | | | | | | | | | | |
| Study Day | 701 | 729 | 757 | 785 | 813 | 841 | 869 | 897 | 925 | 953 | 981 | 1009 | |
| Window (day) | ±5 | ±5 | ±5 | ±5 | ±5 | ±5 | ±5 | ±5 | ±5 | ±5 | ±5 | ±5 | ET¹ |
| ALXN1210 | X | X | X | X | X | X | X | X | X | X | X | X | |
| administration | | | | | | | | | | | | | |
| PNH clone | | | | | | X | | | | | | X | |
| size² | | | | | | | | | | | | | |
| Pregnancy test³ | X | X | X | X | X | X | X | X | X | X | X | X | X |
| PNH | X | X | X | X | X | X | X | X | X | X | X | X | X |
| symptomatology⁴ | | | | | | | | | | | | | |
| Physical | | | | | | | | | | | | | X |
| examination | | | | | | | | | | | | | |
| Abbreviated | X | X | X | X | X | X | X | X | X | X | X | X | |
| physical | | | | | | | | | | | | | |
| examination⁵ | | | | | | | | | | | | | |
| Vital signs | X | X | X | X | X | X | X | X | X | X | X | X | X |
| 12-Lead ECG⁶ | | | | | | | | | | | | X | X |
| Chemistry, | X | X | X | X | X | X | X | X | X | X | X | X | X |
| including LDH | | | | | | | | | | | | | |
| Hematology⁷ | X | X | X | X | X | X | X | X | X | X | X | X | X |
| Coagulation | X | X | X | X | X | X | X | X | X | X | X | X | X |
| Urinalysis and | X | X | X | X | X | X | X | X | X | X | X | X | X |
| urine chemistry | | | | | | | | | | | | | |
| PK sampling⁸ | | | | | | X | | | | | | X | X |
| Serum PD | | | | | | X | | | | | | X | X |
| panel⁹ | | | | | | | | | | | | | |
| Immunogenicity | | | | | | X | | | | | | X | X |
| (ADA)¹⁰ | | | | | | | | | | | | | |
| QoL | | | X | | | X | | | X | | | X | X |
| assessments | | | | | | | | | | | | | |
| Infusion site | X | X | X | X | X | X | X | X | X | X | X | X | |
| evaluation¹¹ | | | | | | | | | | | | | |
| VAS for | X | X | X | X | X | X | X | X | X | X | X | X | |
| infusion site | | | | | | | | | | | | | |
| pain¹² | | | | | | | | | | | | | |

| | | |
|---|---|---|
| Review safety | ← ← ← Review continuously → → → | |
| card | | |
| Concomitant | ← ← ← Monitor continuously → → → | X |
| medications | | |
| Adverse events | ← ← ← Monitor continuously → → → | X |

Abbreviations: ADA = antidrug antibody; ECG = electrocardiogram; ET = early termination; LDH = lactate dehydrogenase; PD = pharmacodynamic; PK = pharmacokinetic; PNH = paroxysmal nocturnal hemoglobinuria; QoL = quality of life; VAS = visual analog scale
¹The ET visit is only performed for patients who discontinue or are withdrawn early from the study.
²Red blood cell clone size only during the Extension Period.
³Female patients of childbearing potential only. Serum pregnancy test end of study (Day 1009) or ET visit only; urine pregnancy test at all other time points. A negative urine test result is required prior to administering ALXN1210 to female patients on dosing days.
⁴Investigator assessment of the following events: fatigue, abdominal pain, dyspnea, dysphagia, chest pain, and erectile dysfunction. Symptoms of disease burden will be captured through the QoL questionnaires.
⁵Abbreviated physical examination consists of a body system relevant examination based upon Investigator judgment and subject symptoms.
⁶Obtain triplicate 12-lead ECGs during the end of study (predose Day 1009) or at ET visit.
⁷Assessment for safety as well as the following parameters as secondary endpoints: free hemoglobin, haptoglobin, reticulocyte count, and D-dimer.
⁸Cohort-specific sampling time points.
⁹Serum for exploratory PD assays; cohort-specific sampling time points.
¹⁰Immunogenicity samples are collected predose on dosing days.
¹¹An induration or reaction of < 10 mm is not listed as an adverse event unless it persists for more than 24 hours, at which time the patient informs the study staff immediately and proceeds to the nearest hospital emergency department.
¹²Patient assess infusion site pain using a 100 mm VAS. The VAS is completed as soon as practical after completion of the infusion.

TABLE 7
Schedule of Assessments: Extension Period and Early Termination: Cohort 2

| | Extension | | | | | | | | | | |
|---|---|---|---|---|---|---|---|---|---|---|---|
| Period | Year 1 | | Year 2 | | | | | | | | |
| Study Day | 295 | 337 | 379 | 421 | 463 | 505 | 547 | 589 | 631 | 673 | 715 |
| Window (day) | ±5 | ±5 | ±5 | ±5 | ±5 | ±5 | ±5 | ±5 | ±5 | ±5 | ±5 |
| ALXN1210 | X | X | X | X | X | X | X | X | X | X | X |
| administration | | | | | | | | | | | |
| PNH clone size² | | X | | | | X | | | | X | |
| Pregnancy test³ | X | X | X | X | X | X | X | X | X | X | X |
| PNH | X | X | X | X | X | X | X | X | X | X | X |
| symptomatology⁴ | | | | | | | | | | | |
| Physical | | | | | | | | | | | |
| examination | | | | | | | | | | | |
| Abbreviated | X | X | X | X | X | X | X | X | X | X | X |
| physical | | | | | | | | | | | |
| examination⁵ | | | | | | | | | | | |
| Vital signs | X | X | X | X | X | X | X | X | X | X | X |
| 12-Lead ECG⁶ | | | | | | | | | | | |
| Chemistry, | X | X | X | X | X | X | X | X | X | X | X |
| including LDH | | | | | | | | | | | |
| Hematology⁷ | X | X | X | X | X | X | X | X | X | X | X |
| Coagulation | X | X | X | X | X | X | X | X | X | X | X |
| Urinalysis and | X | X | X | X | X | X | X | X | X | X | X |
| urine chemistry | | | | | | | | | | | |
| PK sampling⁸ | | X | | | | X | | | | X | |
| Serum PD | | X | | | | X | | | | X | |
| panel⁹ | | | | | | | | | | | |
| Immunogenicity | | X | | | | X | | | | X | |
| (ADA)¹⁰ | | | | | | | | | | | |
| QoL | | X | | X | | X | | X | | X | |
| assessments | | | | | | | | | | | |
| Infusion site | X | X | X | X | X | X | X | X | X | X | X |
| evaluation¹¹ | | | | | | | | | | | |
| VAS for | X | X | X | X | X | X | X | X | X | X | X |
| infusion site | | | | | | | | | | | |
| pain¹² | | | | | | | | | | | |

| | |
|---|---|
| Review safety | ← ← ← Review continuously → → → |
| card | |
| Concomitant | ← ← ← Monitor continuously → → → |
| medications | |
| Adverse events | ← ← ← Monitor continuously → → → |

| | Extension | | | | | | | |
|---|---|---|---|---|---|---|---|---|
| Period | Year 3 | | | | | | | |
| Study Day | 757 | 799 | 841 | 883 | 925 | 967 | 1009 | |
| Window (day) | ±5 | ±5 | ±5 | ±5 | ±5 | ±5 | ±5 | ET¹ |
| ALXN1210 | X | X | X | X | X | X | X | |
| administration | | | | | | | | |
| PNH clone size² | | | X | | | | X | |
| Pregnancy test³ | X | X | X | X | X | X | X | X |
| PNH | X | X | X | X | X | X | X | X |
| symptomatology⁴ | | | | | | | | |
| Physical | | | | | | | | X |
| examination | | | | | | | | |
| Abbreviated | X | X | X | X | X | X | X | |
| physical | | | | | | | | |
| examination⁵ | | | | | | | | |
| Vital signs | X | X | X | X | X | X | X | X |
| 12-Lead ECG⁶ | | | | | | | X | X |
| Chemistry, | X | X | X | X | X | X | X | X |
| including LDH | | | | | | | | |
| Hematology⁷ | X | X | X | X | X | X | X | X |
| Coagulation | X | X | X | X | X | X | X | X |
| Urinalysis and | X | X | X | X | X | X | X | X |
| urine chemistry | | | | | | | | |
| PK sampling⁸ | | | X | | | | X | X |
| Serum PD | | | X | | | | X | X |
| panel⁹ | | | | | | | | |
| Immunogenicity | | | X | | | | X | X |
| (ADA)¹⁰ | | | | | | | | |
| QoL | X | | X | | X | | X | X |
| assessments | | | | | | | | |
| Infusion site | X | X | X | X | X | X | X | |
| evaluation¹¹ | | | | | | | | |
| VAS for | X | X | X | X | X | X | X | |
| infusion site | | | | | | | | |
| pain¹² | | | | | | | | |

| | | |
|---|---|---|
| Review safety | ← ← ← Review continuously → → → | |
| card | | |
| Concomitant | ← ← ← Monitor continuously → → → | X |
| medications | | |
| Adverse events | ← ← ← Monitor continuously → → → | X |

Abbreviations: ADA = antidrug antibody; ECG = electrocardiogram; ET = early termination; LDH = lactate dehydrogenase; PD = pharmacodynamic; PK = pharmacokinetic; PNH = paroxysmal nocturnal hemoglobinuria; QoL = quality of life; VAS = visual analog scale
¹The ET visit is only performed for patients who discontinue or are withdrawn early from the study.
²Red blood cell clone size only during the Extension Period.
³Female patients of childbearing potential only. Serum pregnancy test end of study (Day 1009) or ET visit only; urine pregnancy test at all other time points. A negative urine test result is required prior to administering ALXN1210 to female patients on dosing days.
⁴Investigator assessment of the following events: fatigue, abdominal pain, dyspnea, dysphagia, chest pain, and erectile dysfunction. Symptoms of disease burden will be captured through the QoL questionnaires.
⁵Abbreviated physical examination consists of a body system relevant examination based upon Investigator judgment and subject symptoms.
⁶Obtain triplicate 12-lead ECGs during the end of study (predose Day 1009) or at ET visit.
⁷Assessment for safety as well as the following parameters as secondary endpoints: free hemoglobin, haptoglobin, reticulocyte count, and D-dimer.
⁸Cohort-specific sampling time points.
⁹Serum for exploratory PD assays; cohort-specific sampling time points.
¹⁰Immunogenicity samples are collected predose on dosing days.
¹¹An induration or reaction of < 10 mm is not listed as an adverse event unless it persists for more than 24 hours, at which time the patient informs the study staff immediately and proceeds to the nearest hospital emergency department.
¹²Patient assess infusion site pain using a 100 mm VAS. The VAS is completed as soon as practical after completion of the infusion.

TABLE 8
Schedule of Assessments: Extension Period and Early Termination: Cohort 3

| | Extension | | | | | | | | | | | | | |
|---|---|---|---|---|---|---|---|---|---|---|---|---|---|---|
| Period | Year 1 | | Year 2 | | | | | | Year 3 | | | | | |
| Study Day | 309 | 365 | 421 | 477 | 533 | 589 | 645 | 701 | 757 | 813 | 869 | 925 | 981 | |
| Window (day) | ±5 | ±5 | ±5 | ±5 | ±5 | ±5 | ±5 | ±5 | ±5 | ±5 | ±5 | ±5 | ±5 | ET¹ |
| ALXN1210 | X | X | X | X | X | X | X | X | X | X | X | X | X | |
| administration | | | | | | | | | | | | | | |
| PNH clone size² | X | | | X | | | X | | | X | | | X | |
| Pregnancy test³ | X | X | X | X | X | X | X | X | X | X | X | X | X | X |
| PNH symptomatology⁴ | X | X | X | X | X | X | X | X | X | X | X | X | X | X |
| Physical examination | | | | | | | | | | | | | | X |
| Abbreviated physical | X | X | X | X | X | X | X | X | X | X | X | X | X | |
| examination⁵ | | | | | | | | | | | | | | |
| Vital signs | X | X | X | X | X | X | X | X | X | X | X | X | X | X |
| 12-Lead ECG⁶ | | | | | | | | | | | | | X | X |
| Chemistry, including LDH | X | X | X | X | X | X | X | X | X | X | X | X | X | X |
| Hematology⁷ | X | X | X | X | X | X | X | X | X | X | X | X | X | X |
| Coagulation | X | X | X | X | X | X | X | X | X | X | X | X | X | X |
| Urinalysis and urine | X | X | X | X | X | X | X | X | X | X | X | X | X | X |
| chemistry | | | | | | | | | | | | | | |
| PK sampling⁸ | X | | | X | | | X | | | X | | | X | X |
| Serum PD panel⁹ | X | | | X | | | X | | | X | | | X | X |
| Immunogenicity (ADA)¹⁰ | X | | | X | | | X | | | X | | | X | X |
| QoL assessments | | X | | X | | X | | X | | X | | X | X | X |
| Infusion site evaluation¹¹ | X | X | X | X | X | X | X | X | X | X | X | X | X | |
| VAS for infusion site pain¹² | X | X | X | X | X | X | X | X | X | X | X | X | X | |

| | | |
|---|---|---|
| Review safety card | ← ← ← Review continuously → → → | |
| Concomitant medications | ← ← ← Monitor continuously → → → | X |
| Adverse events | ← ← ← Monitor continuously → → → | X |

Abbreviations: ADA = antidrug antibody; ECG = electrocardiogram; ET = early termination; LDH = lactate dehydrogenase; PD = pharmacodynamic; PK = pharmacokinetic; PNH = paroxysmal nocturnal hemoglobinuria; QoL = quality of life; VAS = visual analog scale
¹The ET visit is only performed for patients who discontinue or are withdrawn early from the study.
²Red blood cell clone size only during the Extension Period.
³Female patients of childbearing potential only. Serum pregnancy test end of study (Day 1009) or ET visit only; urine pregnancy test at all other time points. A negative urine test result is required prior to administering ALXN1210 to female patients on dosing days.
⁴Investigator assessment of the following events: fatigue, abdominal pain, dyspnea, dysphagia, chest pain, and erectile dysfunction. Symptoms of disease burden will be captured through the QoL questionnaires.
⁵Abbreviated physical examination consists of a body system relevant examination based upon Investigator judgment and subject symptoms.
⁶Obtain triplicate 12-lead ECGs during the end of study (predose Day 1009) or at ET visit.
⁷Assessment for safety as well as the following parameters as secondary endpoints: free hemoglobin, haptoglobin, reticulocyte count, and D-dimer.
⁸Cohort-specific sampling time points.
⁹Serum for exploratory PD assays; cohort-specific sampling time points.
¹⁰Immunogenicity samples are collected predose on dosing days.
¹¹An induration or reaction of < 10 mm is not listed as an adverse event unless it persists for more than 24 hours, at which time the patient informs the study staff immediately and proceeds to the nearest hospital emergency department.
¹²Patient assess infusion site pain using a 100 mm VAS. The VAS is completed as soon as practical after completion of the infusion.

TABLE 1
Schedule of Assessments: Extension Period and Early Termination: Cohort 4

| | Extension | | | | | | | | | | |
|---|---|---|---|---|---|---|---|---|---|---|---|
| Period | Year 1 | | Year 2 | | | | Year 3 | | | | |
| Study Day | 281 | 365 | 449 | 533 | 617 | 701 | 785 | 869 | 953 | 1037 | |
| Window (day) | ±5 | ±5 | ±5 | ±5 | ±5 | ±5 | ±5 | ±5 | ±5 | ±5 | ET¹ |
| ALXN1210 | X | X | X | X | X | X | X | X | X | X | |
| administration | | | | | | | | | | | |
| PNH clone size² | | X | | X | | X | | X | | X | |
| Pregnancy test³ | X | X | X | X | X | X | X | X | X | X | X |
| PNH | X | X | X | X | X | X | X | X | X | X | X |
| symptomatology⁴ | | | | | | | | | | | |
| Physical | | | | | | | | | | | X |
| examination | | | | | | | | | | | |
| Abbreviated | X | X | X | X | X | X | X | X | X | X | |
| physical | | | | | | | | | | | |
| examination⁵ | | | | | | | | | | | |
| Vital signs | X | X | X | X | X | X | X | X | X | X | X |
| 12-Lead ECG⁶ | | | | | | | | | | X | X |
| Chemistry, | X | X | X | X | X | X | X | X | X | X | X |
| including LDH | | | | | | | | | | | |
| Hematology⁷ | X | X | X | X | X | X | X | X | X | X | X |
| Coagulation | X | X | X | X | X | X | X | X | X | X | X |
| Urinalysis and | X | X | X | X | X | X | X | X | X | X | X |
| urine chemistry | | | | | | | | | | | |
| PK sampling⁸ | X | | X | | X | | X | | | X | X |
| Serum PD | X | | X | | X | | X | | | X | X |
| panel⁹ | | | | | | | | | | | |
| Immunogenicity | X | | X | | X | | X | | | X | X |
| (ADA)¹⁰ | | | | | | | | | | | |
| QoL | X | | X | | X | | X | | | X | X |
| assessments | | | | | | | | | | | |
| Infusion site | X | X | X | X | X | X | X | X | X | X | |
| evaluation¹¹ | | | | | | | | | | | |
| VAS for | X | X | X | X | X | X | X | X | X | X | |
| infusion | | | | | | | | | | | |
| site pain¹² | | | | | | | | | | | |

| | | |
|---|---|---|
| Review safety | ← ← ← Review continuously → → → | |
| card | | |
| Concomitant | ← ← ← Monitor continuously → → → | X |
| medications | | |
| Adverse events | ← ← ← Monitor continuously → → → | X |

Abbreviations: ADA = antidrug antibody; ECG = electrocardiogram; ET = early termination; Abbreviations: ADA = antidrug antibody; ECG = electrocardiogram; ET = early termination; LDH = lactate dehydrogenase; PD = pharmacodynamic; PK = pharmacokinetic; PNH = paroxysmal nocturnal hemoglobinuria; QoL = quality of life; VAS = visual analog scale
¹The ET visit is only performed for patients who discontinue or are withdrawn early from the study.
²Red blood cell clone size only during the Extension Period.
³Female patients of childbearing potential only. Serum pregnancy test end of study (Day 1009) or ET visit only; urine pregnancy test at all other time points. A negative urine test result is required prior to administering ALXN1210 to female patients on dosing days.
⁴Investigator assessment of the following events: fatigue, abdominal pain, dyspnea, dysphagia, chest pain, and erectile dysfunction. Symptoms of disease burden will be captured through the QoL questionnaires.
⁵Abbreviated physical examination consists of a body system relevant examination based upon Investigator judgment and subject symptoms.
⁶ Obtain triplicate 12-lead ECGs during the end of study (predose Day 1009) or at ET visit.
⁷Assessment for safety as well as the following parameters as secondary endpoints: free hemoglobin, haptoglobin, reticulocyte count, and D-dimer.
⁸Cohort-specific sampling time points.
⁹Serum for exploratory PD assays; cohort-specific sampling time points.
¹⁰Immunogenicity samples are collected predose on dosing days.
¹¹An induration or reaction of < 10 mm is not listed as an adverse event unless it persists for more than 24 hours, at which time the patient informs the study staff immediately and proceeds to the nearest hospital emergency department.
¹²Patient assess infusion site pain using a 100 mm VAS. The VAS is completed as soon as practical after completion of the infusion.

A patient can withdraw from the study at any time at his/her own request, or can be withdrawn at any time at the discretion of the Investigator. Patients who discontinue dosing re instructed to return for follow-up visits, unless they withdraw consent and/or are lost to follow-up.

If the patient withdraws consent the Early Termination visit is completed as soon as possible. If the patient is taken off of ALXN1210 the early termination visit should occur prior to initiation of complement therapy.

If the patient is withdrawn from ALXN1210, the patient should return for the remainder of the protocol visits until starting a different complement-targeted therapy Patients are permanently discontinued from ALXN1210 treatment if any of the following occur during the study: (a) Serious infusion reaction (such as bronchospasm with wheezing or requiring ventilator support or symptomatic hypotension) or serum sickness-like reactions manifesting 1 to 14 days after drug administration, (b) severe uncontrolled infection, (c) pregnancy or planned pregnancy, or (d) if the Investigator deem it is in the best interest of the patient.

5. Treatment of Patients

Management of Potential Drug Infusion Reactions: some patients treated with IV infusions of mAbs have experienced concurrent infusion-related reactions with signs or symptoms that can be classified as acute allergic reactions/hypersensitivity reactions or cytokine release syndrome. The signs and symptoms include headache, fever, facial flushing, pruritus, myalgia, nausea, chest tightness, dyspnea, vomiting, erythema, abdominal discomfort, diaphoresis, shivers, hypertension, lightheadedness, hypotension, palpitations, and somnolence. Anaphylaxis might occur at any time during an infusion and patients will be monitored closely prior to and through 1 hour following the end of the infusion of ALXN1210. All adverse events which may indicate an infusion-related response are graded according to criteria from the Common Terminology Criteria for Adverse Events (CTCAE) v4.0.3.

Before infusion is started, the treating physician and other appropriate personnel, medication (adrenaline, inhaled beta agonists, antihistamines, and corticosteroids), and other requirements to treat anaphylaxis must be readily available.

The infusion is stopped immediately if ≥Grade 2 allergic/hypersensitivity reactions (including drug fever) or ≥Grade 3 cytokine release syndrome/acute infusion reaction occurs. Patients experiencing a reaction during the administration of study drug should be treated according to institutional guidelines.

For a Grade 1 or Grade 2 infusion-related reaction, the infusion is stopped and medication with antihistamine (e.g., with diphenhydramine, 25 to 50 mg orally or equivalent) and acetaminophen (650 mg orally or equivalent) may be considered. If the signs and symptoms have resolved with the above medications, the infusion is restarted. If the infusion is slowed, the total infusion time should not exceed 5 hours, including any interruptions for safety or technical reasons. The study drug is stopped if the infusion reaction recurs. Patients experiencing an infusion reaction are observed in the clinic until resolution of the reaction.

If an event of anaphylaxis occurs, according to the criteria in Table 4 then subcutaneous epinephrine ( 1/1000, 0.3 to 0.5 mL or equivalent) is considered. In the case of bronchospasm, inhaled beta agonist is considered. Patients administered antihistamine for the study drug or prevention of infusion reactions are given appropriate warnings about drowsiness and impairment of driving ability prior to discharge.

Patients who experience a severe reaction during administration of study drug resulting in discontinuation of study drug undergo all scheduled safety, PK, and PD evaluations required by the protocol.

TABLE 10
Clinical Criteria for Diagnosing Anaphylaxis
Anaphylaxis is highly likely when any one of the following 3 criteria are fulfilled:

| | |
|---|---|
| 1. | Acute onset of an illness (minutes to several hours) with involvement of the skin, |
| | mucosal tissue, or both (e.g., generalized hives, pruritus or flushing, swollen lips- |
| | tongue-uvula) |

AND AT LEAST ONE OF THE FOLLOWING:

| | a. | Respiratory compromise (e.g., dyspnea, wheeze-bronchospasm, stridor, reduced PEF, |
|---|---|---|
| | | hypoxemia) |
| | b. | Reduced BP or associated symptoms of end-organ dysfunction (e.g., hypotonia |
| | | [collapse], syncope, incontinence) |
| 2. | Two or more of the following that occur rapidly after exposure to a likely allergen for that | |
| | patient (minutes to several hours): | |
| | a. | Involvement of the skin-mucosal tissue (e.g., generalized hives, itch-flush, swollen |
| | | lips-tongue-uvula) |
| | b. | Respiratory compromise (e.g., dyspnea, wheeze-bronchospasm, stridor, reduced PEF, |
| | | hypoxemia) |
| | c. | Reduced BP or associated symptoms (e.g., hypotonia [collapse], syncope, |
| | | incontinence) |
| | d. | Persistent gastrointestinal symptoms (e.g., crampy abdominal pain, vomiting) |
| 3. | Reduced BP after exposure to known allergen for that patient (minutes to several hours): | |
| | a. | Systolic BP of less than 90 mmHg or greater than 30% decrease from that person's |
| | | baseline |

Source:
Adapted from Sampson HA, et al., Second symposium on the definition and management of anaphylaxis: summary report: Second National Institute of Allergy and Infectious Disease/Food Allergy and Anaphylaxis Network symposium. J Allergy Clin Immunol. 2006; 117(2): 391-397.
PEF, Peak expiratory flow;
BP, blood pressure.

Infection Risk: Due to its mechanism of action, the use of ALXN1210 increases the patient's susceptibility to meningococcal infection (N meningitidis). Patients might be at risk of disease by uncommon serogroups (such as X), although meningococcal disease due to any serogroup may occur. To reduce the risk of infection, all patients are vaccinated prior to receiving ALXN1210. Patients who are treated with ALXN1210 less than 2 weeks after receiving a meningococcal vaccine receive treatment with appropriate prophylactic antibiotics until 2 weeks after vaccination. Vaccines against serotypes A, C, Y, W 135, and B, where available, are recommended to prevent common pathogenic meningococcal serotypes. Patients must be vaccinated or revaccinated according to current national vaccination guidelines or local practice for vaccination use with complement inhibitors (e.g., eculizumab).

Vaccination may not be sufficient to prevent meningococcal infection. Consideration should be given per official guidance and local practice on the appropriate use of antibacterial agents. All patients are monitored for early signs of meningococcal infection, evaluated immediately if infection is suspected, and treated with appropriate antibiotics, if necessary.

To increase risk awareness and promote quick disclosure of any potential signs or symptoms of infection experienced by the patients during the course of the study, patients are provided a safety card to carry with them at all times.

Prior and Concomitant Medications and Procedures: Prior medications (including vitamins and herbal preparations), including those discussed in the exclusion criteria and/or procedures (any therapeutic intervention, such as surgery/biopsy or physical therapy) that the patient takes or undergoes within 28 days (or 3 years for documentation of meningococcal vaccination) prior to signing the informed consent form (ICF) until the first dose of ALXN1210 are recorded on the patient's electronic case report form (eCRF). All medication used during screening and the Treatment and Extension periods are recorded in the patient's source/chart and electronic case report form. This record includes all prescription drugs, herbal products, vitamins, minerals, over-the-counter medications, and current medications for PNH. Any changes in concomitant medications are recorded in the patient's source/chart and electronic case report form. Any concomitant medication deemed necessary for the patient's standard of care treatment during the study, or for the treatment of any adverse event, along with those the allowed medications described is given at the discretion of the Investigator.

The following concomitant medications are allowed if the following apply, and dose adjustments are not expected during the treatment period:
Patients are taking erythropoietin on a stable dose for at least 8 weeks prior to screening.
Patients are taking immunosuppressants on a stable dose for at least 8 weeks prior to screening.
Patients are taking corticosteroids on a stable dose for at least 4 weeks prior to screening.
Patients are allowed to take vitamin K antagonists (e.g., warfarin) but must have had a stable international normalized ratio (INR) level (per investigator discretion) for 4 weeks prior to screening.
Patients are taking iron supplements or folic acid on a stable dose for at least 4 weeks prior to screening.
Patients are allowed to take low molecular weight heparin on a stable dose for at least 4 weeks prior to screening.
   Adjustments in the frequency or level of dosing in any of the above medications are made if it is in the best interest of the patient.

Treatment Compliance: Patients are administered ALXN1210 in a controlled setting under the Investigator's supervision, thereby ensuring compliance with ALXN1210 administration. Study coordinators at the investigative site ensure that all patients are adequately informed on the specific ALXN1210 dosing regimen required for compliance with the study protocol.

Randomization and Blinding. This is an open-label study. Up to 26 patients are enrolled in the study. The first 2 eligible patients who meet the inclusion/exclusion criteria are assigned to Cohort 1. The DMC conducts a review of the available safety data and determines whether the next cohort is opened. If additional patients are screened and are eligible for enrollment before a dose-escalation decision has been made by the DMC for any cohort, those patients are assigned to the active cohort with the lowest dose level

4. Study Drug Materials and Management

Each vial of study drug contains 150 mg of ALXN1210 in 10 mM sodium phosphate, 150 mM sodium chloride, 0.02% polysorbate 80, and Water for IV administration. ALXN1210 is formulated at pH 7.0 and is presented as a sterile, preservative-free, 10 mg/mL solution for IV administration, supplied in 20-mL single-use vials. ALXN1210 is suitable for human use and manufactured under current Good Manufacturing Practices (GMP).

TABLE 11
Investigational Product

| | Investigational Product |
|---|---|
| Product Name: | ALXN1210 |
| Dosage Form: | Sterile solution for IV administration |
| Unit Dose | 150 mg/vial |
| Route of Administration | Intravenous |
| Manufacturer | Alexion Pharmaceuticals, Inc. |

ALXN1210 is supplied in a one-vial-per-kit configuration. Each vial and carton is labeled according to specific country or region regulatory requirements. ALXN1210 vials are stored in refrigerated conditions at 2° C. to 8° C. (36° F. to 46° F.) and protected from light. ALXN1210 vials are not frozen or shaken. Preparation of ALXN1210 doses is performed in accordance with site-specific local standards by qualified and study-trained pharmacy personnel. Handling and preparation of materials used to prepare and administer study drug is carried out using aseptic techniques for sterile products.

Pharmacy personnel prepare doses in accordance with the dose assignment. ALXN1210 is diluted in 0.9% sodium chloride injection (country-specific pharmacopeia) and administered by IV infusion at a fixed rate, as indicated in Table 12:

TABLE 12
Dosing Reference Chart for ALXN1210 Dose Preparation

| | | Maximum | Minimum |
|---|---|---|---|
| | | Infusion Rate | Infusion Duration |
| Dose (mg) | Infusion Volume (mL) | (mL/hour) | minutes (hours) |
| 1000 | 200 | 333 | 36.0 (0.6) |
| 1400 | 280 | 333 | 50.4 (0.8) |
| 1600 | 320 | 333 | 57.6 (1.0) |
| 2000 | 400 | 333 | 72.1 (1.2) |
| 2400 | 480 | 333 | 86.4 (1.4) |
| 3000 | 600 | 333 | 108.1 (1.8) |
| 5400* | 1080 | 333 | 194.6 (3.2) |

*To be given in divided dose.
Please refer to the Pharmacy Manual for additional dose preparation instructions. These dosing rates apply to patients ≥50 kg; for patients <50 kg, please refer to the pharmacy manual.

For in-use shelf life, ALXN1210 is diluted with 0.9% sodium chloride injection (country-specific pharmacopeia) before administration (dosing solution). The dosing solution is stable for 6 hours at room temperature 15° C. to 25° C. (59° F. to 77° F.) and for 24 hours at 2° C. to 8° C. (36° F. to 46° F.). The expiration date and time of the dosing solution is calculated from the time dose preparation is complete. The dose is administered within the expiration date and time.

All doses of ALXN1210 are administered by IV infusion, using a programmable IV infusion pump and IV sets with in-line filters at an infusion rate up to 333 mL/hr.

During the induction and maintenance periods (up to Day 253), patients remain seated or semi-reclined for the time of drug administration and remain in the clinic for an additional 2 hours for safety observations. Time of dosing (t=0) is defined as ALXN1210 start of infusion (SOI). All procedures are performed in relation to SOI or EOI as described in the Schedules of Assessments.

The study site must maintain accurate records demonstrating dates and amount of study drug received, to whom dispensed (patient-by-patient accounting), and accounts of any study drug accidentally or deliberately destroyed. Accountability logs are provided to assist the pharmacist in maintaining current and accurate inventory records covering receipt, dispensing, and disposition of the study drug. The study monitor examines the inventory during the study. Accountability records are readily available and may be subject to regulatory authorities, the local regulatory agency, or an independent auditor's inspection at any time.

Unless otherwise notified, empty vials and vials with residual materials are kept for inspection and accountability by the study monitor prior to their destruction or handled per local site pharmacy standard operating procedures for clinical study drugs. At the end of the study, a final reconciliation is made between the amount of study drug supplied, dispensed, and subsequently destroyed or returned. A written explanation is provided for any discrepancies.

6. Pharmacokinetic Assessments

Blood Sample Collection: the total volume of blood collected per patient for clinical laboratory, PK, PD, and immunogenicity assessments does not exceed 300 mL in any 16-week period.

After ALXN1210 administration, blood samples for determination of serum ALXN1210 concentrations are collected at the time points indicated in the Schedule of Assessments, with the actual blood sampling dates and times being recorded and used in PK calculations. The timing of PK sample collection is altered based on initial PK results to ensure appropriate PK monitoring. The number of PK sampling time points for any given patient does not exceed the currently planned number of time points.

Serum concentrations of ALXN1210 and blood samples for analyses of C5 levels (total and free), cRBC hemolysis, and quantitative measures of C5 activation are collected at the time points specified in Table 13. Serum samples are stored for additional PK/PD analyses.

TABLE 13
Collection Time Points for Serum Concentrations of ALXN1210 and Analysis
of Hemolytic Activity

| Cohort | Period | Dose Number and Study Day | Collection Windows |
|---|---|---|---|
| 1 | Treatment | Dose 1 on Day 1 | Predose (up to 0.5 hour |
| | | Dose 2 on Day 15 | before) |
| | | | EOI (up to 0.5 hour after) |
| | | | 4 hours post-SOI (±1 hour) |
| | | | Day 22 (±1 day) |
| | | Doses 3 to 11 on Days 29, 57, | Predose (up to 0.5 hour |
| | | 85, 113, 141, 169, 197, 225, | before) |
| | | and 253 | EOI (up to 0.5 hour after) |
| | | | 4 hours post-SOI (±1 hour) |
| | | | Day 43 (±1 day) |
| | | | Days 127 and 211 (±2 days) |
| | | | Day 253 (up to 0.5 hour |
| | | | before Dose 11) |
| | Extension | Doses 14, 20, 26, 32, and 38 on | Predose (up to 0.5 hour |
| | | Days 337, 505, 673, 841, and | before) |
| | | 1009 | When a patient discontinues |
| | | | the study |
| 2 | Treatment | Dose 1 on Day 1 | Predose (up to 0.5 hour |
| | | Dose 2 on Day 22 | before) |
| | | | EOI (up to 0.5 hour after) |
| | | | 4 hours post-SOI (±1 hour) |
| | | | Day 29 (±1 day) |
| | | Doses 3 to 8 on Days 43, 85, | Predose (up to 0.5 hour |
| | | 127, 169, 211, and 253 | before) |
| | | | EOI (up to 0.5 hour after) |
| | | | 4 hours post-SOI (±1 hour) |
| | | | Day 57 (±1 day) |
| | | | Days 113, 141, 197, and 225 |
| | | | (±2 days) |
| | | | Day 253 (up to 0.5 hour |
| | | | before Dose 8) |
| | Extension | Doses 10, 14, 18, 22, and 26 on | Predose (up to 0.5 hour |
| | | Days 337, 505, 673, 841, and | before) |
| | | 1009 | When a patient discontinues |
| | | | the study |
| 3 | Treatment | Dose 1 on Day 1 | Predose (up to 0.5 hour |
| | | Dose 2 on Day 15 | before) |
| | | | EOI (up to 0.5 hour after) |
| | | | 4 hours post-SOI (±1 hour) |
| | | | Days 22 (±1 day) |
| | | Doses 3 to 7 on Days 29, 85, | Predose (up to 0.5 hour |
| | | 141, 197, and 253 | before) |
| | | | EOI (up to 0.5 hour after) |
| | | | 4 hours post-SOI (±1 hour) |
| | | | Days 43 and 57 (±1 day) |
| | | | Days 113, 127, 169, 211, and |
| | | | 225 (±2 days) |
| | | | Day 253 (up to 0.5 hour |
| | | | before Dose 7) |
| | Extension | Doses 8, 11, 14, 17, and 20 on | Predose (up to 0.5 hour |
| | | Days 309, 477, 645, 813, and | before) |
| | | 981 | When a patient discontinues |
| | | | the study |
| 4 | Treatment | Dose 1 on Day 1 | Predose (up to 0.5 hour |
| | | | before) |
| | | | EOI (up to 0.5 hour after) |
| | | | 4 hours post-SOI (±1 hour) |
| | | | Day 15 (±1 day) |
| | | Doses 2 to 4 on Days 29, 113, | Predose (up to 0.5 hour |
| | | and 197 | before) |
| | | | EOI (up to 0.5 hour after) |
| | | | 6 hours post-SOI (±1 hour) |
| | | | Day 57 (±1 day) |
| | | | Days 141 and 225 (±2 days) |
| | | | Day 253 |
| | Extension | Doses 5, 7, 9, 11, and 13 on | Predose (up to 0.5 hour |
| | | Days 281, 449, 617, 785, and | before) |
| | | 1037 | When a patient discontinues |
| | | | the study |

Abbreviations:
EOI = end of infusion;
SOI = start of infusion

7. Immunogenicity Assessments

Blood samples for the assessment of antidrug antibody (ADA) to ALXN1210 are collected at the time points specified in Table 14:

TABLE 14
Collection Time Points for Serum Samples for Immunogenicity
Analyses of Antidrug Antibodies to ALXN1210

| | |
|---|---|
| Cohort 1 | Predose on Days 1, 15, 29, 57, 85, 113, 141, 169, 197, 225, |
| | 253, 337, 505, 673, 841, and 1009 or ET |
| Cohort 2 | Predose on Days 1, 22, 43, 85, 127, 169, 211, 253, 337, 505, |
| | 673, 841, and 1009 or ET |
| Cohort 3 | Predose on Days 1, 15, 29, 85, 141, 197, 253, 309, 477, 645, |
| | 813, and 981 or ET |
| Cohort 4 | Predose on Days 1, 29, 113, 197, 281, 449, 617, 785, |
| | and 1037 or ET |

8. Assessment of Efficacy

Blood samples for chemistry are used to measure Lactate Dehydrogenase (LDH) levels at the time points described in the Schedule of Assessments.

Biomarkers of PNH: A serum PD panel is collected for analyses of C5 levels (total and free), cRBC hemolysis, and quantitative measures of C5 activation at the time points indicated in Table 7. In addition, serum samples are stored for potential additional analyses.

Hemolysis-related hematological parameters are assessed by measurements of free hemoglobin, haptoglobin, reticulocyte count, PNH RBC clone size (%), and D-dimer. Hematology assessments are obtained at the time points described in the Schedule of Assessments.

Investigator assessment of clinical symptoms related to PNH is made at the time points described in the Schedule of Assessments. The Investigator assesses patients for the following events: fatigue, abdominal pain, chest pain, dyspnea, dysphasia, and erectile dysfunction. Symptoms of disease burden are captured through the quality of life (QoL) questionnaire.

Quality of Life: The FACIT-F scale (Version 4.0) is a collection of quality of life (QoL) questionnaires targeted to the management of fatigue symptoms due to a chronic illness. The European Organization for Research and Treatment of Cancer, Quality of Life Questionnaire-Core 30 Scale, Version 3.0 (EORTC scale) is a questionnaire developed to assess the QoL of cancer patients. Both scales are administered at the time points described in the Schedule of Assessments.

Patients are assessed for the following events: fatigue, abdominal pain, dyspnea, dysphagia, chest pain, and erectile dysfunction. Symptoms of disease burden are captured through the QoL questionnaire. Assessment of clinical symptoms related to PNH is made at the time points specified in the Schedule of Assessments.

Markers of PNH symptoms and comorbidities (i.e., chronic kidney disease by urinary spot albumin:creatinine ratio and (estimated glomerular filtration rate) eGFR, and (brain natriuretic peptide) BNP for pulmonary hypertension) is evaluated in the study as exploratory efficacy endpoints. Evaluation for changes in kidney function is based on investigator assessment and laboratory results of serum and urinary creatinine and eGFR. The estimated glomerular filtration rate is calculated using the Modification of Diet in Renal Disease formula at the same time blood is drawn for chemistry assessments (specified in the Schedule of Assessments).

Major adverse vascular events (MAVE) are assessed as part of the planned evaluation for adverse events. The definition of a MAVE is provided below. The description of event, location, method of diagnosis (magnetic resonance imaging [MRI], ultrasound, angiogram, or other), date of diagnosis and date resolved (or ongoing) will be collected on the electronic case report form as part of the patient's medical history and during the study.

A MAVE can be only of the following events:
Thrombophlebitis/deep vein thrombosis
Pulmonary embolus
Myocardial infarction
Transient ischemic attack
Unstable angina
Renal vein thrombosis/renal artery thrombosis/glomerular thrombosis, renal infarction
Acute peripheral vascular occlusion
Mesenteric/visceral vein/arterial thrombosis or infarction
Hepatic/portal vein thrombosis
Cerebral arterial occlusion/cerebrovascular accident
Cerebral venous occlusion
Renal arterial thrombosis
Multi-infarct dementia
Other, specify

9. Assessment of Efficacy

Patients meet with the Investigator or designee to discuss the potential safety risks of ALXN1210 and to allow for the Investigator to address any of the patient's safety concerns at the time points shown in the Schedule of Assessments.

Collection of adverse events (including serious adverse events (serious adverse events) and MAVEs) are monitored from the time informed consent is obtained until study completion. Investigators follow any adverse events through to their conclusion. In the event of patient discontinuation from the study, adverse event monitoring continues through the last study visit, if possible. Clinical and laboratory assessments are performed to assess ALXN1210 safety. Timing of the assessments is described in the Schedule of Assessments. Any abnormal results are followed until resolution or stabilization.

A review of demographic parameters, including age, gender, race, and ethnicity is performed, as described in the Schedule of Assessments. A complete medical history is taken and documented.

Weight, height, and body mass index are recorded, as described in the Schedule of Assessments. A physical examination assessing general appearance, skin, head/eyes/ears/nose/throat, neck, lymph nodes, chest, heart, abdominal cavity, limbs, central nervous system, and musculoskeletal is performed at the time points specified in the Schedule of Assessments.

Vital signs are taken after the patient has been resting in the supine position for at least 5 minutes, and include temperature (° C.; oral), respiratory rate, supine blood pressure, and pulse. The timing of vital sign assessments is described in the Schedule of Assessments. Out-of-range blood pressure or pulse measurements are repeated at the Investigator's discretion. Any confirmed, clinically significant vital sign measurements are recorded as adverse events.

An electrocardiogram (ECG) is obtained after the patient has been resting in a supine position for at least 5 minutes. A triplicate 12-lead ECG is obtained at screening, prior to the first dose of ALXN1210 on Day 1, and at the end of study (Day 1009) or Early Termination visit. A single ECG is obtained at the other time points specified in the Schedule of Assessments. Heart rate, PR, QRS, RR, and QT are measured, and corrected QTcF intervals (Fridericia's formula) are calculated.

Serum pregnancy testing (beta human chorionic gonadotropin) is performed in all female patients at screening and end of study/Early Termination. Urine pregnancy testing (with a minimum human chorionic gonadotropin detection limit of 25 IU/L) is performed at the time points specified in the Schedule of Assessments.

Blood samples for analysis of hematology, chemistry, coagulation, urinalysis/urine chemistry, virus serology and other parameters are collected as described in the Schedule of Assessments. Abnormal results are followed, as appropriate.

Blood samples are analyzed for chemistry parameters. Considering that indirect bilirubin is calculated from total and direct bilirubin values, indirect bilirubin results are not be available if direct bilirubin is below the limit of quantification. Serum FSH levels and estradiol concentrations are measured at screening for postmenopausal female patients to confirm their postmenopausal status. Timing of chemistry assessments is described in the Schedule of Assessments.

Urine samples are analyzed. Urine samples are also analyzed to measure proteins and creatinine to calculate the urine protein:creatinine ratio. Timing of urinalysis and urine chemistry assessments is described in the Schedule of Assessments.

Virus Serology: Blood samples are analyzed and performed at the time points specified in the Schedule of Assessments.

Brain Natriuretic Protein: Blood samples for BNP analysis re performed at the time points specified in the Schedule of Assessments.

Evaluation for infusion site reaction is made at the time point described in Schedule of Assessments. Administration site reactions are recorded as an adverse event using the appropriate coding terms.

An induration or reaction of <1 cm is not listed as an adverse event unless it persists for more than 24 hours. Pain at site of infusion is assessed using a 100 mm visual assessment analog scale (VAS).

10. Adverse Event Management

The Investigator is responsible for detecting, assessing, documenting and reporting all adverse events. All adverse events are recorded from the signing of informed consent until study completion. There is no time limit for severe adverse events that are considered causally related.

All observed or volunteered adverse events, regardless of causal relationship, must be reported and recorded in the elecetronic case report form. Adverse events reported by the patient and/or parent or legal guardian, and/or identified in response to an open-ended question from study personnel, or revealed by observation, physical examination, or other study procedures are collected and recorded.

An adverse event is defined as any unfavorable and unintended sign (e.g., including an abnormal laboratory finding), symptom, or disease temporally associated with the use of a medicinal product or procedure, whether or not considered related to the medicinal product or procedure, which occurs during the course of the clinical study. Exacerbations of a chronic or intermittent pre-existing condition, including either an increase in frequency and/or intensity of the condition, are all to be considered adverse events.

Abnormal test findings may be considered adverse events. If an abnormal laboratory value is identified, Investigators are encouraged to report a diagnosis, or a sign or symptom, rather than an isolated abnormal test value. An abnormal test finding should be documented as an adverse event if any of the following conditions are met:
Is associated with a sign or symptom
Requires additional diagnostic testing (repeat tests are not considered additional testing)
Requires a medical or surgical intervention
Leads to a change in study dosing outside of the protocol-defined dosing or leads to discontinuation from the study
Requires significant additional treatment
Does not meet any of the conditions above; however, the Investigator or Sponsor considers the result clinically significant or meeting the definition of an adverse event.
   This definition also includes the signs or symptoms resulting from:
Drug overdose
Drug withdrawal
Drug misuse
Drug interactions
Extravasation
Exposure during pregnancy
Exposure via breastfeeding
Medication error
Occupational exposure

An adverse event does not necessarily include the following:
Medical or surgical procedures (e.g., surgery, endoscopies, tooth extraction, transfusion); the condition that leads to the procedure is the AE (e.g., laparoscopic cholecystectomy is the procedure or treatment for an serious adverse event of necrotic gall bladder)
Pre-existing diseases or conditions present or detected prior to the screening evaluation that do not worsen
Situations where an untoward medical occurrence has not occurred (e.g., hospitalization for elective surgery if planned prior to the start of the study, social and/or convenience admissions)

Any adverse event that fulfills any one of the criteria listed below is recorded as a serious adverse event. A serious adverse event is described as any untoward medical occurrence that, at any dose:
Results in death
Is life threatening
Requires hospitalization or prolongation of hospitalization^{b}. Hospitalization does not necessarily include the following:
   Rehabilitation/hospice/nursing facility
   Emergency Room visit less than 24 hours
   Elective or preplanned admission/surgery/day surgery
   Protocol-specified admission
   Admission for a pre-existing condition not associated with either a new adverse event or with worsening of a pre-existing adverse event
Results in persistent or significant disability/incapacity
Is a congenital anomaly/birth defect
Is an important medical event

The term “life threatening” in the definition of “serious” refers to an event in which the patient was at risk of death at the time of the event. It does not refer to an event which hypothetically might have caused death if it were more severe.

Hospitalization requires inpatient admission or prolongation of an existing hospitalization. The adverse events that are associated with hospitalization or prolongation of hospitalization are considered serious adverse events.

Important medical event: Medical and scientific judgment is exercised in deciding whether expedited reporting is appropriate in other situations, such as important medical events that may not be immediately life threatening, or result in death or hospitalization, but may jeopardize the patient or may require intervention to prevent 1 of the other outcomes listed in the definition above. These are also usually be considered serious. Examples of such events are intensive treatment in an emergency room or at home for allergic bronchospasm; blood dyscrasias or convulsions that do not result in hospitalization; or development of drug dependency or drug abuse.

Severity and seriousness are differentiated. Severity describes the intensity of an adverse event, while the term seriousness refers to an adverse event that has met the criteria for a serious adverse event, as described above.

All adverse events re graded according to criteria from CTCAE v4.03, published Jun. 14, 2010.
Grade 1: Mild (awareness of sign or symptom, but easily tolerated)
Grade 2: Moderate (discomfort sufficient to cause interference with normal activities)
Grade 3: Severe (incapacitating, with inability to perform normal activities)
Grade 4: Life threatening
Grade 5: Fatal

Changes in the severity of an adverse event are documented to allow an assessment of the adverse event duration at each level of intensity to be evaluated. Adverse events characterized as intermittent require documentation of onset and duration of each episode, if the severity of the intermittent event changes.

An Investigator causality assessment is provided for all adverse events (both nonserious and serious). This assessment is recorded in the electronic case report form and on any additional forms, as appropriate. The definitions for the causality assessments are as follows:
Not related (unrelated): This relationship suggests that there is no association between the investigational product and the reported event.
Unlikely related: This relationship suggests that the clinical picture is highly consistent with a cause other than the investigational product, but attribution cannot be made with absolute certainty, and a relationship between the investigational product and adverse event cannot be excluded with complete confidence.
Possibly related: This relationship suggests that treatment with the investigational product may have caused or contributed to the adverse event, ie, the event follows a reasonable temporal sequence from the time of drug administration, and/or follows a known response pattern to the investigational product, but could also have been produced by other factors.
Probably related: This relationship suggests that a reasonable temporal sequence of the event with the investigational product administration exists, as well as the likely association of the event with the investigational product. This will be based upon the known pharmacological action of the investigational product, known or previously reported adverse reactions to the investigational product or class of drugs, or judgment based on the Investigator's clinical experience.
Definitely related: Temporal relationship to the investigational product. Other conditions (concurrent illness, concurrent medication reaction, or progression/expression of disease state) do not appear to explain event, corresponds with the known pharmaceutical profile, improvement on discontinuation, reappearance on rechallenge.

For all adverse events, regardless of causal relationships, the Investigator must follow up regarding the outcome of the event until the event or sequelae either resolve or stabilize. Adverse event outcomes must be recorded in the electronic case report form and on any additional forms, as appropriate.

If a patient experiences a serious adverse event with an outcome of death, the following procedures are to be performed:
The serious adverse event resulting in death has an outcome documented as death/fatal, with an end date being the date of death.
If the patient had additional adverse event/serious adverse events that were ongoing at the time of death, these events are documented as ongoing with no end date.
Only 1 event has an outcome of death/fatal, unless an autopsy report or

Investigator states otherwise.

All observed or volunteered adverse events, regardless of dose cohort or causal relationship, are reported. For all adverse events, the Investigator must do the following:

1. Determine the adverse event outcome

2. Determine if the event meets criteria for a serious adverse event

3. Assess adverse event severity

4. Determine adverse event causality

Adverse events are documented in clear, unambiguous medical terms. Study personnel are advised not to use abbreviations or acronyms.

For each adverse event, only the diagnosis is recorded on the electronic case report.

Characteristic signs and symptoms of the diagnosis are not reported as additional adverse events. If a diagnosis is not available, each sign and symptom is recorded as an adverse event. When a diagnosis becomes available, the source document and the electronic case report art updated with the relevant diagnosis only.

For medical or surgical procedures (e.g., surgery, endoscopies, tooth extraction, transfusion), the condition/diagnosis that leads to the procedure is recorded as the adverse event (e.g., laparoscopic cholecystectomy is the procedure or treatment for an serious adverse event of necrotic gall bladder).

All adverse events that later increase in frequency and or severity (medical and scientific judgment should be exercised by the Investigator) are considered new adverse events, and re recorded on a new line in the electronic case report form.

Withdrawal due to an adverse event or serious adverse event is clearly differentiated from withdrawal due to other reasons.

All adverse events are assessed by the Investigator to determine if they meet criteria for a serious adverse event. All serious adverse events are reported immediately, or within 24 hours of the Investigator and/or study site staff becoming aware of the event, regardless of the presumed relationship to the study drug.

Pregnancy data is collected during the study for all patients. Exposure during pregnancy, also called exposure in utero, can be the result of either maternal exposure or transmission of drug product via semen following paternal exposure. Exposure during pregnancy is recorded and followed. If a female patient participating in this study, or a male patient's female partner becomes or is found to be pregnant while being treated or exposed to study drug, the Investigator submits the “Pregnancy Reporting and Outcome/Breastfeeding Form” Female patients who become pregnant are discontinued from dosing, but are followed for safety where feasible. Male patients may continue in the study if an accidental pregnancy of their female partner occurs, despite adequate contraception.

Pregnancy in itself is not regarded as an adverse event unless there is a suspicion that investigational product may have interfered with the effectiveness of a contraceptive medication. However, complications of pregnancy and abnormal outcomes of pregnancy are adverse events, and many may meet criteria for an SAE. Complications of pregnancy and abnormal outcomes of pregnancy, such as ectopic pregnancy, spontaneous abortion, intrauterine fetal demise, neonatal death, or congenital anomaly, meet the criteria of a serious adverse event and therefore should be reported as a serious adverse event. Elective abortions without complications are not handled as an adverse event.

11. Statistics

All data collected is documented using summary tables, figures, and data listings. For categorical variables, frequencies and percentages are presented for each cohort, and for the combined cohorts. For continuous variables, descriptive statistics (n, mean, median, SD, minimum, maximum) are presented for each cohort, and for the combined cohorts.

Descriptive statistics for PK parameters include the number of observations, mean, SD, coefficient of variance (CV), median, minimum, maximum, geometric mean, and geometric % CV.

A clinical study report (CSR) is produced after the end of the maintenance period and includes safety, efficacy, PK, and PD analyses. A final CSR is produced at study completion and includes data on all patients in the study at the end of the extension period.

The Safety Set consists of all patients who received at least 1 dose of ALXN1210. This population is used for the safety analysis.

The full analysis set (FAS) consista of all patients in the safety population who had a baseline LDH measurement and at least 1 postbaseline LDH measurement. The FAS is used for all efficacy analyses.

The PK population consists of all patients who have sufficient serum concentration data to enable the calculation of PK parameters.

The immunogenicity analysis population consists of all patients who have both a predose and postdose ADA sample collected.

A sample size of 15 patients from the combined cohorts provides approximately 90% power to detect a mean paired difference in LDH from baseline of −40% at Day 253, with an estimated SD of 45%. This is based on a 2-sided paired t-test, with 5% type I error rate. To account for a possible 15% dropout rate, 18 patients are enrolled.

All patients are included in the summaries of disposition, which summarizes the number of patients randomized in the study, the frequency and percentage of patients who completed or discontinued from the study, along with reason for discontinuation, by cohort. Demographics and baseline characteristics are summarized for all patients by each cohort and overall.

Safety analyses are performed on the Safety Set, which consists of all patients who receive at least 1 dose of ALXN1210, and are reported by cohort and overall. Safety analyses include all AEs, ECGs, clinical laboratory data, physical examinations, and vital sign measurements, and are presented using descriptive statistics. No inferential statistical analyses are planned on safety parameters. The incidence of treatment-emergent adverse events and SAEs are summarized by System Organ Class and Preferred Term for each cohort and overall, by severity, and by relationship to ALXN1210. Adverse events are coded using the Medical Dictionary for Regulatory Activities, Version 18.0 or higher. Serious AEs and AEs resulting in withdrawal from the study are listed. Patients having multiple AEs within a category (e.g., overall, System Orhan Class, Preferred Term) are counted once in that category. For severity tables, a patient's most severe event within a category are counted.

Changes from baseline in vital signs and laboratory assessments (chemistry, complete blood count with differential, and urinalysis) are summarized by cohort. Shift tables of clinical laboratory tests (Low, Normal, High) by cohort are produced. Graphical displays are presented, as appropriate.

The ECG parameters are measured at the specified time points, including heart rate, PR, RR, QRS, QT, and corrected QTcF intervals. The average of the triplicate ECG readings at the time points collected are calculated, and changes from pretreatment baseline values are assessed by cohort.

All concomitant medications are coded using the World Health Organization Drug Dictionary, and the frequency and percentage of concomitant medications will be summarized.

Efficacy analyses are performed on the Full Analysis Set (FAS), which include the Safety Set subset with a baseline and at least 1 postbaseline LDH measurement.

Absolute LDH levels, and the change and percent change from baseline are summarized at all study visits. Baseline is defined as the average of all available assessments on or prior to the first ALXN1210 infusion. A mixed model for repeated measures (MMRM) with the fixed, categorical effect of visit and fixed, continuous effect of baseline LDH levels as covariates is fit to test whether changes and percent changes differ from zero at each time point. An unstructured covariance analysis is used to model the within-patient errors. If this analysis fails to converge, the following structures are tested and the final covariance structure is determined by Akaike's information criterion: first-order autoregressive, compound symmetry, and Toeplitz method. The Kenward-Roger approximation is used to estimate denominator degrees of freedom. As a sensitivity analysis, changes and percent changes from baseline are analyzed, using the Wilcoxon signed-rank test. Graphical displays are presented, as appropriate.

The percentage of patients with clinical symptoms is summarized for all study visits. Changes in hematologic measures are similarly analyzed using an MMRM and Wilcoxon signed-rank test. Scoring guidelines for the FACIT-Fatigue and EORTC scales are used to calculate Quality of Life (QoL) scores. Changes from baseline in FACIT-Fatigue and EORTC scale scores are summarized descriptively for all study visits, and analyzed using MMRM and Wilcoxon signed rank test. Transfusion rates and the incidence rate of MAVEs are summarized.

Individual serum concentration data for ALXN1210-treated patients, with actual sampling dates and times, are used to derive the pharmacokinetic (PK) parameters by noncompartmental analyses, using Phoenix® WinNonlin® (Pharsight Corporation, St Louis, Mo.) Version 6.3 or higher.

The following PK parameters are estimated: maximum observed serum concentration (Cₘₐₓ) after each dose, time to maximum observed serum concentration (tₘₐₓ), minimum observed serum concentration (Cₘᵢₙ), the observed serum concentration at the end of the dosage interval τ (C_{trough}), area under the serum concentration-versus-time curve from time 0 (dosing) to the last quantifiable concentration (AUCₜ), area under the concentration-versus-time curve from time 0 (dosing) to the end of the dosing interval (AUCₜ), apparent terminal-phase elimination rate constant (λ_{z}), terminal elimination half-life (t_{1/2}), and, if possible, total clearance (CL) and volume of distribution at steady state (Vₛₛ). Attainment of steady state and accumulation at steady state also are determined. Dose proportionality and time linearity in PK parameters are assessed.

Mean serum ALXN1210 concentrations versus nominal time and individual serum ALXN1210 concentrations versus actual time are graphically presented. Descriptive statistics (mean, SD, CV, median, minimum, maximum, geometric mean, and geometric % CV) of the serum concentration and PK parameter summaries are provided, as appropriate.

The pharmacodynamic (PD) effects of ALXN1210 administered IV are evaluated by assessing changes and percent changes in serum total and/or free C5 concentrations, cRBC hemolysis. Assessments of PK-PD relationships are explored using data from this study or in combination with data from other studies. Immunogenicity, as measured by antidrug antibodies, is summarized in tabular form by treatment.

### Example 7: Interim Results from Phase 2, Open-Label, Multiple Ascending Dose, Dose-Escalation Study in PNH Patients

The following is a summary of interim data from an ongoing open-label, multiple ascending dose escalation study in PNH patients treated for 36 weeks, which was conducted substantially according to the protocol described above in Example 6. Four cohorts of patients 18 years of age or older having confirmed PNH diagnosis by high-sensitivity flow cytometry and a mean LDH≥3 times the upper limit of normal (ULN) were investigated. Key efficacy endpoints include monitoring changes in: (1) LDH levels from baseline to Day 253, (2) hemolysis-related parameters, and (3) clinical manifestations in PNH.

The baseline demographics, safety profiles, and transfusion history of the patients are set forth in Tables 14, 15, and 16, respectively.

TABLE 14
Demographics

| | | Cohort 1 | Cohort 2 | Cohort 3 | |
|---|---|---|---|---|---|
| | | (1000 mg | (1600 mg | (2400 mg | |
| | | Q4wk)# | Q6wk)# | Q8wk)# | Total |
| Visit | Statistics | N = 6 | N = 6 | N = 7 | N = 19 |
| Race, | | | | | |
| n (%) | | | | | |
| Asian | n (%) | 0 (0) | 0 (0) | 4 (57) | 4 (21) |
| Black or | n (%) | 0 (0) | 0 (0) | 0 (0) | 0 (0) |
| African | | | | | |
| American | | | | | |
| White | n (%) | 5 (83) | 4 (67) | 3 (43) | 12 (63) |
| Not | n (%) | 1 (17) | 2 (33) | 0 (0) | 3 (16) |
| Reported | | | | | |
| Gender | | | | | |
| Male | n (%) | 4 (67) | 5 (83) | 6 (86) | 15 (79) |
| Female | n (%) | 2 (33) | 1 (17) | 1 (14) | 4 (21) |
| Treatment | n | 6 | 6 | 7 | 19 |
| Duration | Mean (SD) | 3.1 (0.48) | 1.4 (0.01) | 0.9 (0.03) | 1.8 (0.98) |
| (months) | Median | 2.8 | 1.4 | 0.9 | 1.4 |
| | Min, Max | 2.8, 3.8 | 1.4, 1.4 | 0.9, 1.0 | 0.9, 3.8 |

#maintenance doses

TABLE 15
Safety Profile

| | Cohort 1 | Cohort 2 | Cohort 3 | |
|---|---|---|---|---|
| | (1000 mg | (1600 mg | (2400 mg | |
| Event Type | Q4wk)# | Q6wk)# | Q8wk)# | Overall |
| (Numbers of Patients) | (N = 6) | (N = 6) | (N = 7) | (N = 19) |
| No. Patients with | 0 | 1 | 1 | 2 |
| Serious TEAEs | | | | |
| | | Neisseria | 2 events | |
| | | Meningitidis | (headache | |
| | | (D57, Grade 4, | and Nausea, | |
| | | resolved) | D2, Grade 1). | |
| | | | Headache | |
| | | | resolved, | |
| | | | nausea | |
| | | | resolved with | |
| | | | sequelae | |
| No. Patients with | 6 (100.0) | 5 (83.3) | 5 (71.4) | 16 (84.2) |
| TEAEs | | | | |
| No. Patients with | 2 (33.3) | 2 (33.3) | 2 (28.6) | 6 (31.6) |
| Related TEAEs | | | | |
| Preferred Term for | Patient 1 | Patient 1 | Patient 1 | |
| Related TEAE | Thrombop | Neutropenia | Headache | |
| | helbitis | (D14, Grade | (D1, Grade 2, | |
| | (Day 8, | 1; D23 Grade 2, | resolved) | |
| | Grade 2, | both resolved) | Patient 2 | |
| | not | Patient 2 | Skin lesion, | |
| | resolved) | Headache (D1, | D3, Grade 1, | |
| | Patient 2 | D23 Grade 1, | resolved | |
| | Headache | resolved) | | |
| | (D1, D15, | Myalgia (D1, | | |
| | resolved) | Grade 1, | | |
| | | resolved) | | |
| | | Asthenia, | | |
| | | Grade 2, | | |
| | | unknown | | |
| | | outcome) | | |

#maintenance doses

TABLE 16
Tranfusion History

| | | Cohort 1 | Cohort 2 | Cohort 3 | |
|---|---|---|---|---|---|
| | | (1000 mg | (1600 mg | (2400 mg | |
| | | Q4wk)# | Q6wk)# | Q8wk)# | Overall |
| Parameter | Statistics | n = 6 | n = 6 | N = 7 | n = 19 |
| Patients with | n (%) | 0 | 2 (33.3) | 3 (42.9) | 5 (26.3) |
| Transfusion Within | | | [4 transf., | [14 | [18 transf., |
| One Year Prior to First | | | 6 units] | transf., | 35 units] |
| ALXN1210 dose | | | | 29 units] | |
| [No. transfusions, | | | | | |
| Total units transfused] | | | | | |
| Patients with | n (%) | 0 | 1 (16.7) | 2 (28.6) | 3 (15.8) |
| Transfusions Since | | | [5 transf., | [2 transf., | [7 transf., |
| First ALXN1210 dose | | | 8 units] | 4 units] | 12 units] |
| [Total units | | | | | |
| transfused] | | | | | |

#maintenance doses
Note:
Patients receiving transfusions after ALXN1210 are the same patients who received transfusions prior to ALXN1210

ALXN1210 shows an acceptable safety profile up to 2400 mg. As shown in Table 15, there were two serious TEAEs. The most common treatment-emergent adverse event (TEAE) was headache. Most TEAE's were unrelated to treatment.

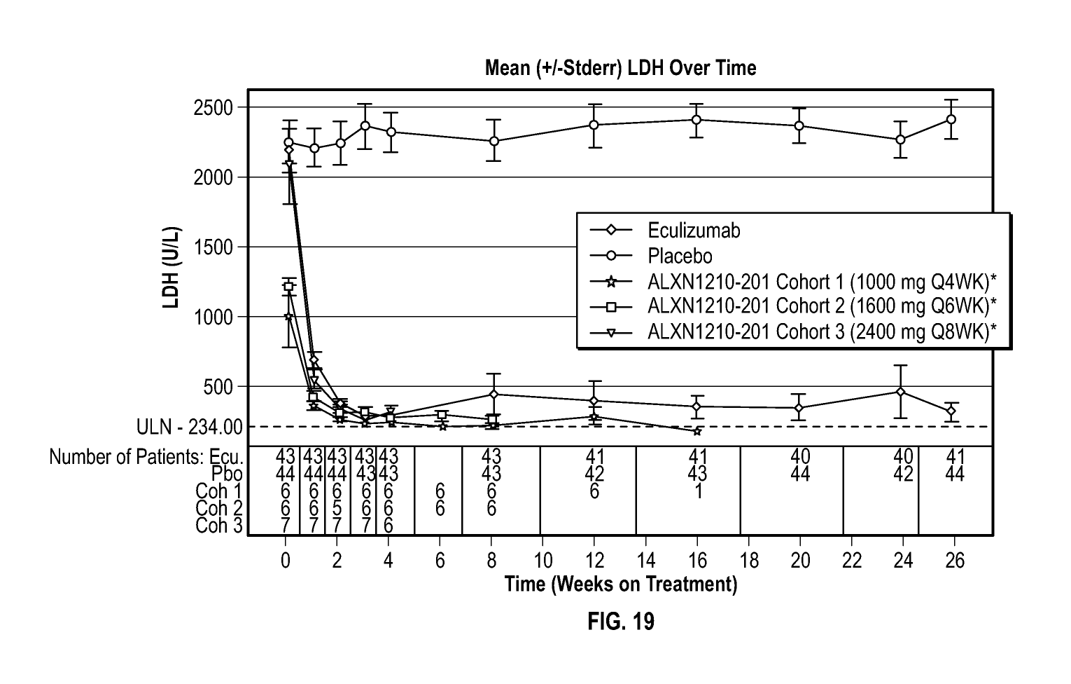

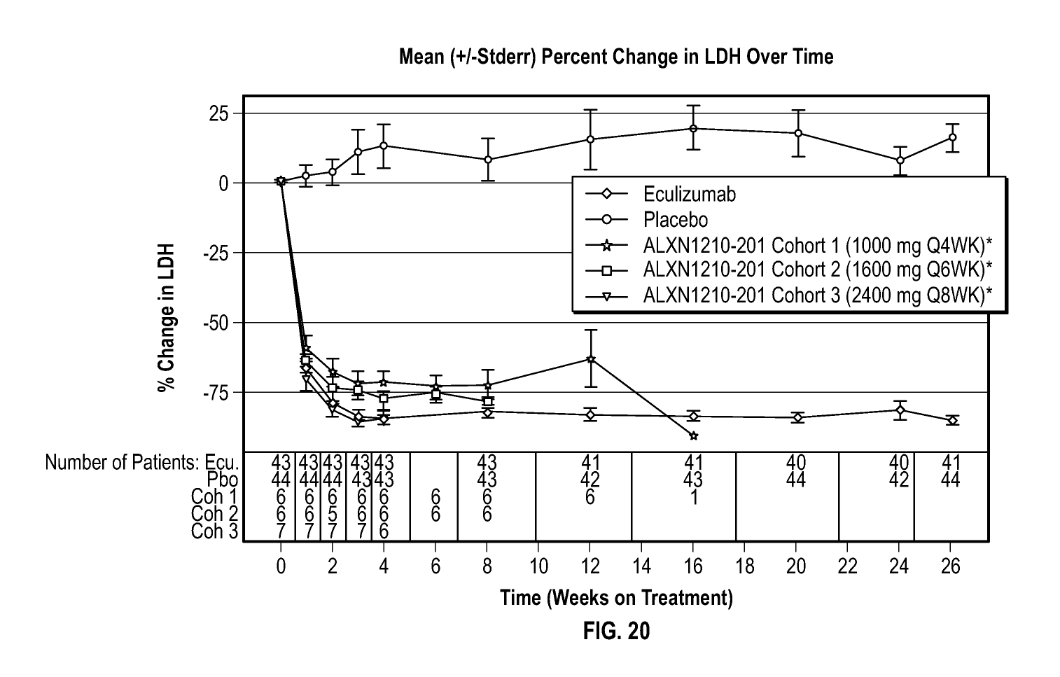

As shown in FIGS. 19 and 20, all patients experienced at least a 40% reduction in LDH compared to baseline. The overall mean reduction on Day 7 was −64.9% (−58.9%, −64.7%, −70.3% for cohorts 1,2,3 respectively). Among 5 patients with history (<1 year prior to ALXN1210) of transfusion, 3 subjects had transfusion while receiving ALXN1210 (1 in cohort 2 and 2 in cohort 3).

As shown in FIG. 20, patients on ALXN1210 were showing further reductions in LDH as compared to a historical comparison to eculizumab. Mostly having their LDH levels lowered to below what is considered the ULN, whereas eculizmab tended to reduce the LDH level to near the ULN but still remained above the ULN.

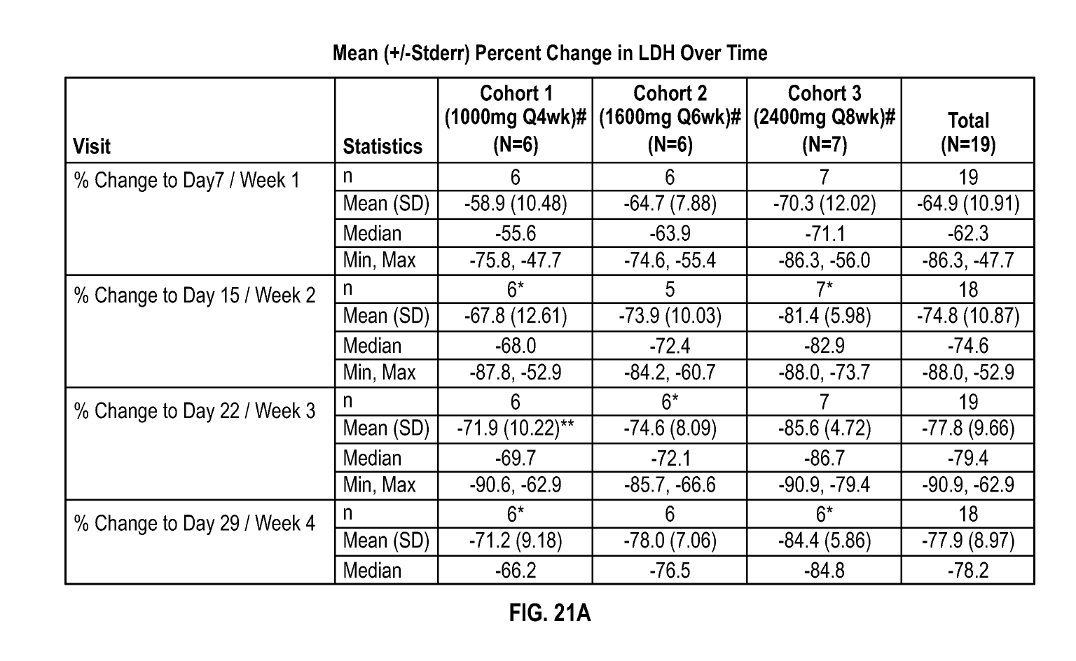

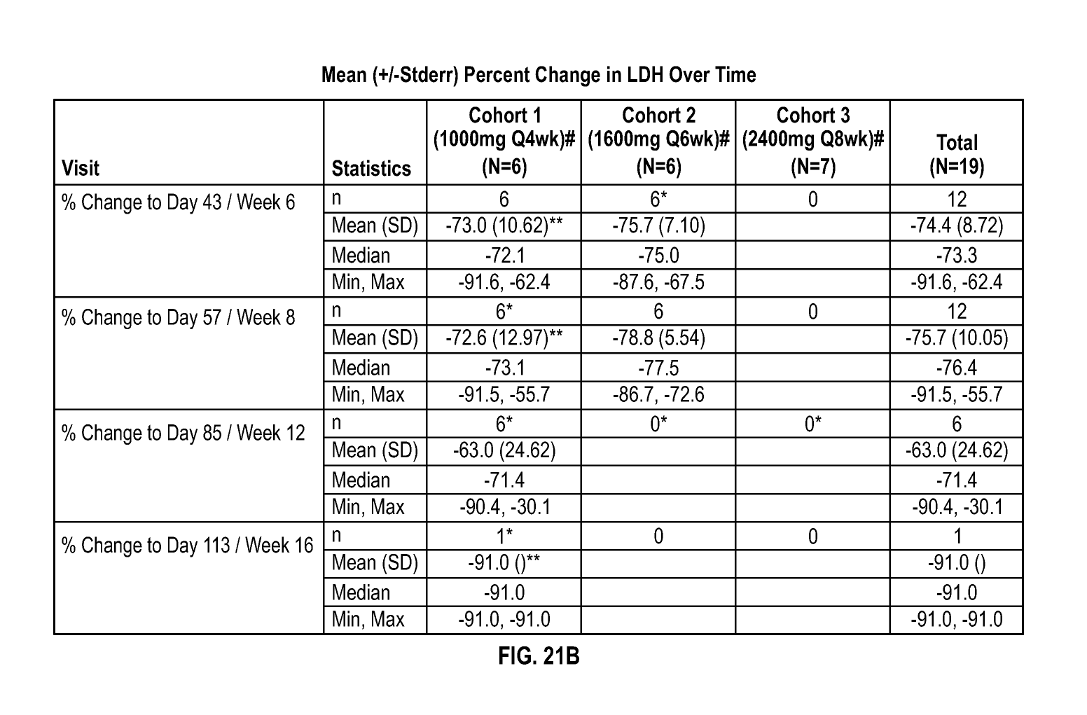

FIGS. 21A-4B set forth the raw mean, median, and minimum/maximum percentage change in LDH levels from baseline data for patients in Cohorts 1, 2, and 3 after treatment with ALXN1210 from Week 1 through Week 4 (FIG. 21A) and from Week 6 through Week 16 (FIG. 21B). These preliminary results show a rapid and sustained LDH reduction in response to ALXN1210.

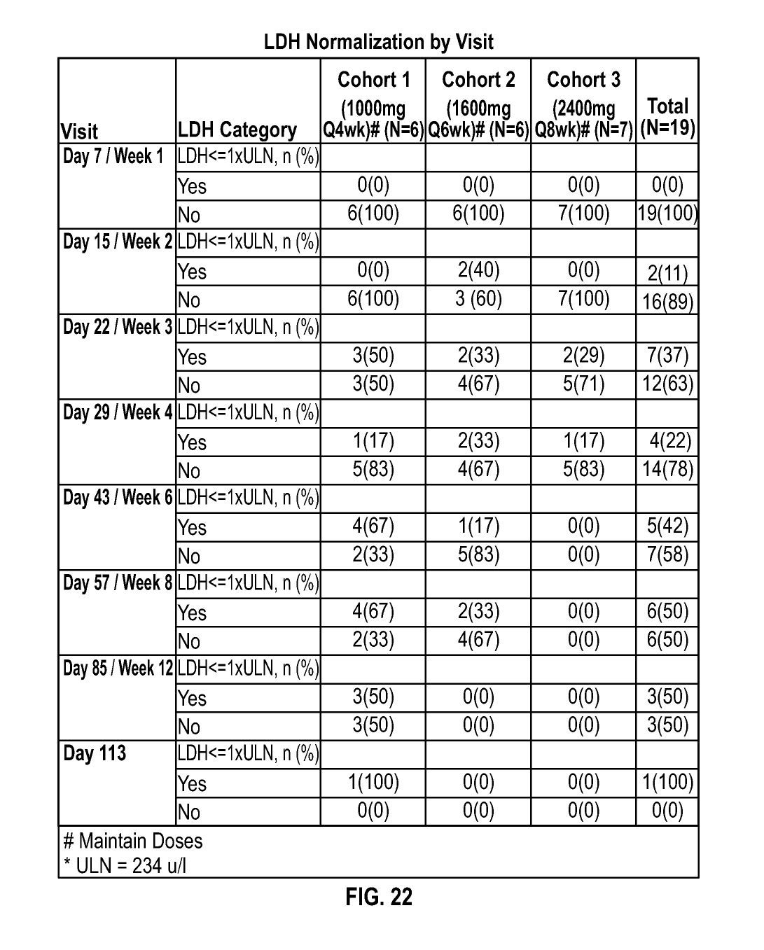

FIG. 22 is the raw mean LDH normalization data for patients in Cohorts 1, 2, and 3 after treatment with ALXN1210 through Day 113.

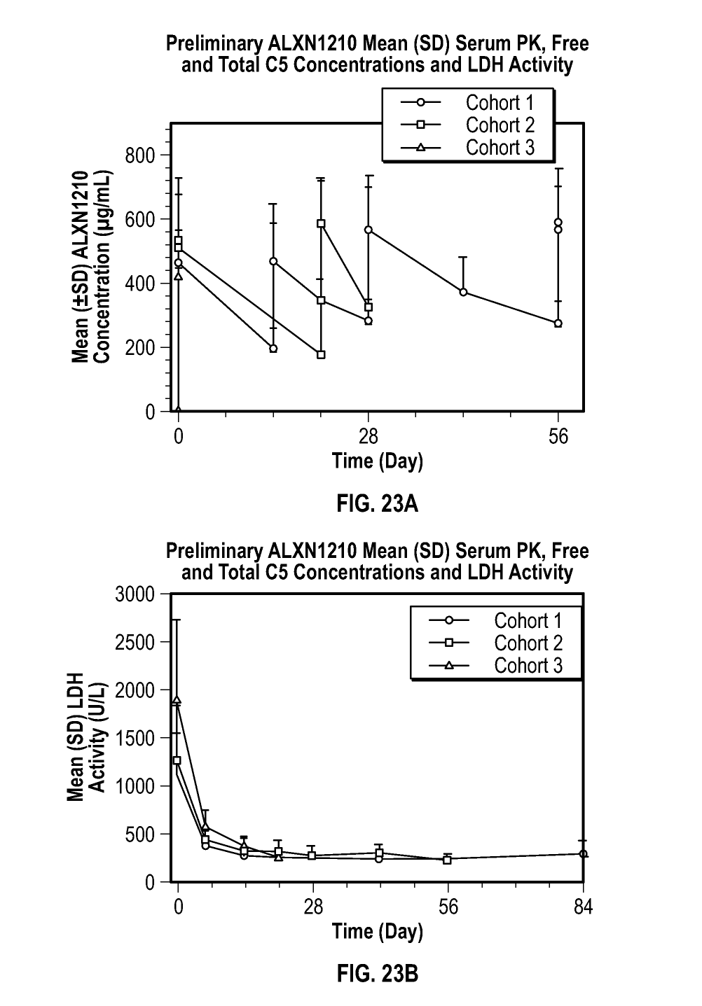

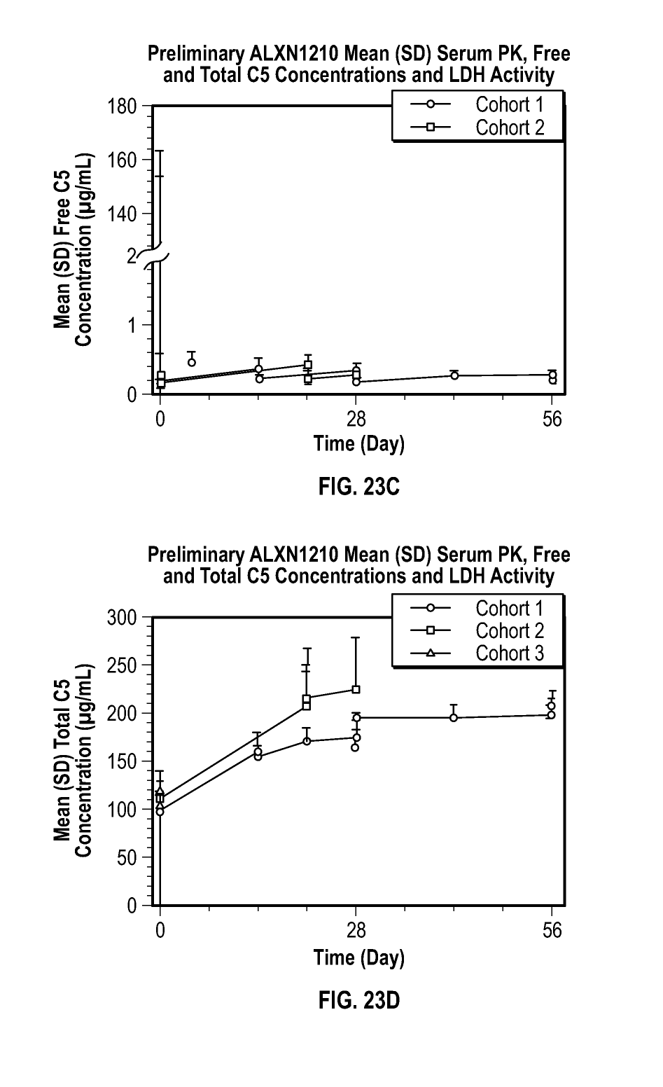

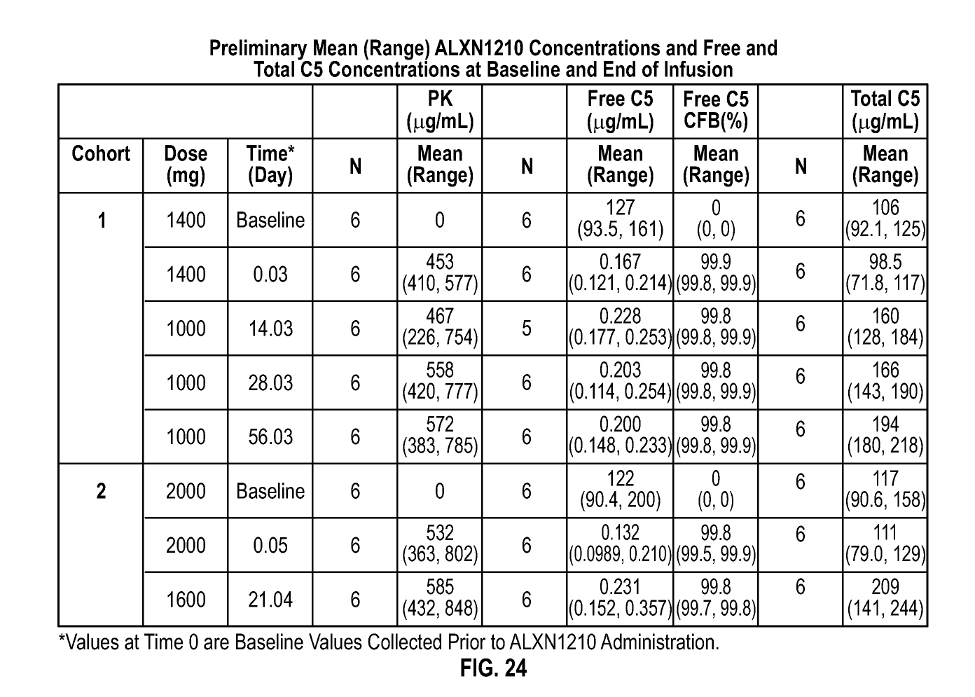

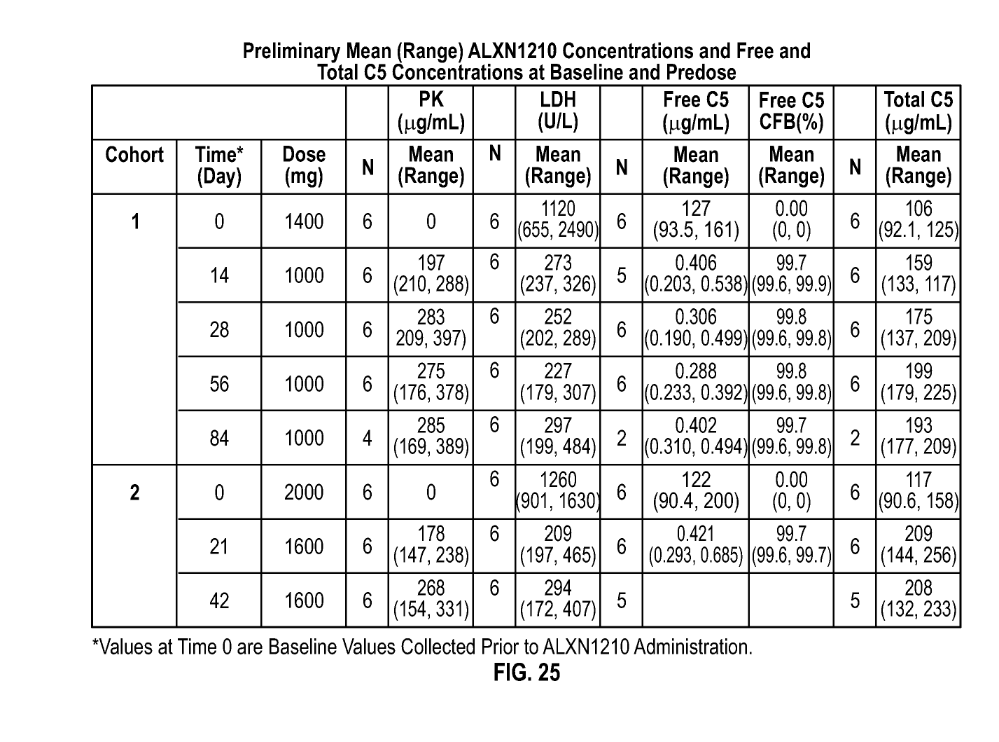

FIG. 23 displays preliminary serum PK, free and total C5 concentrations, and LDH activity following multiple dose administration in PNH patients. FIGS. 24 and 25 summarize the preliminary mean (range) ALXN1210 concentration, LDH activity and free and total concentrations at EOI (FIG. 24) and pre-dose (FIG. 25). Following ALXN1210 administration in PNH patients, immediate, complete and sustained terminal complement inhibition was achieved, as evidenced by reduced serum free C5 by EOI and at predose, respectively. The preliminary range of mean % CFB in free C5 concentrations was 99.8 to 99.9% and 99.8 to 99.8% at EOI for Cohorts 1 and 2, respectively (data not shown). While the preliminary range of mean percent change from baseline % CFB in free C5 concentrations was 99.7 to 99.8% and 99.7 at predose for Cohorts 1 and 2, respectively.

Preliminary immunogenicity results show that all patients in the study were ADA negative through Day 43 (data not shown).

### Example 8: Further Interim Data from Phase 2, Open-Label, Multiple Ascending Dose, Dose-Escalation Study in PNH Patients

The following is a summary of further interim data from the ongoing open-label, multiple-dose, multi-center intrapatient dose-escalation study conducted substantially according to the protocol described above in Example 6, which supplements the results described in Example 7.

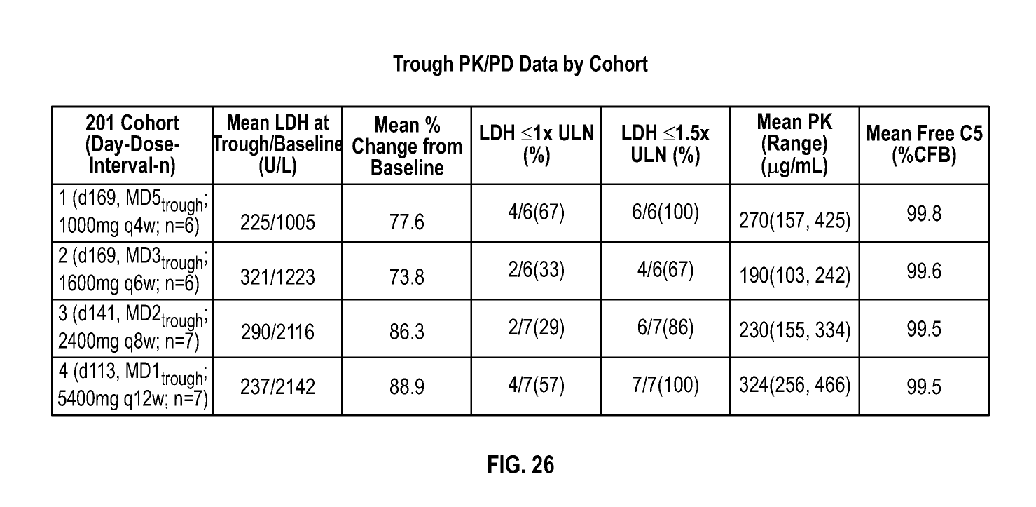

FIG. 26 is a summary of the trough pharmacokinetic and pharmacodynamic data by cohort through Day 113 (Cohort 4), Day 141(Cohort 3) and Day 169 (Cohorts 1 and 2).

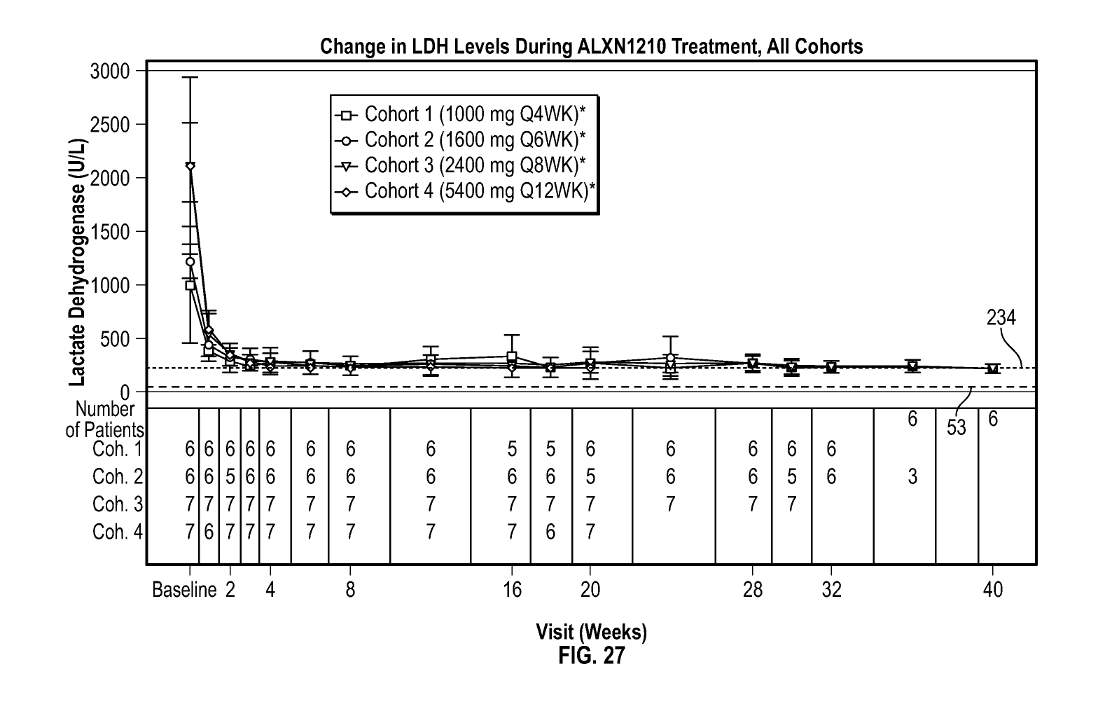

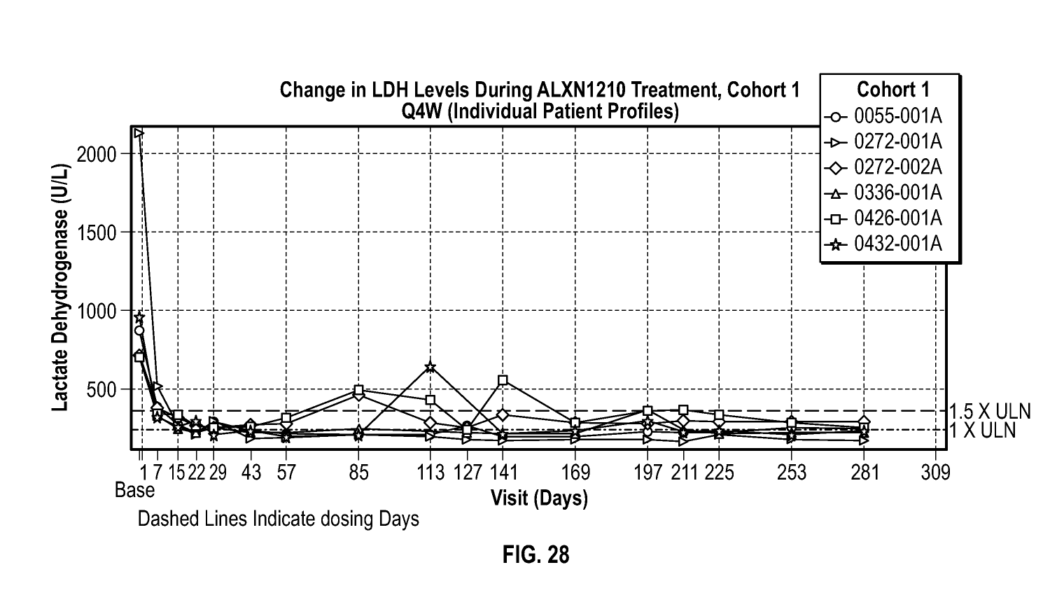

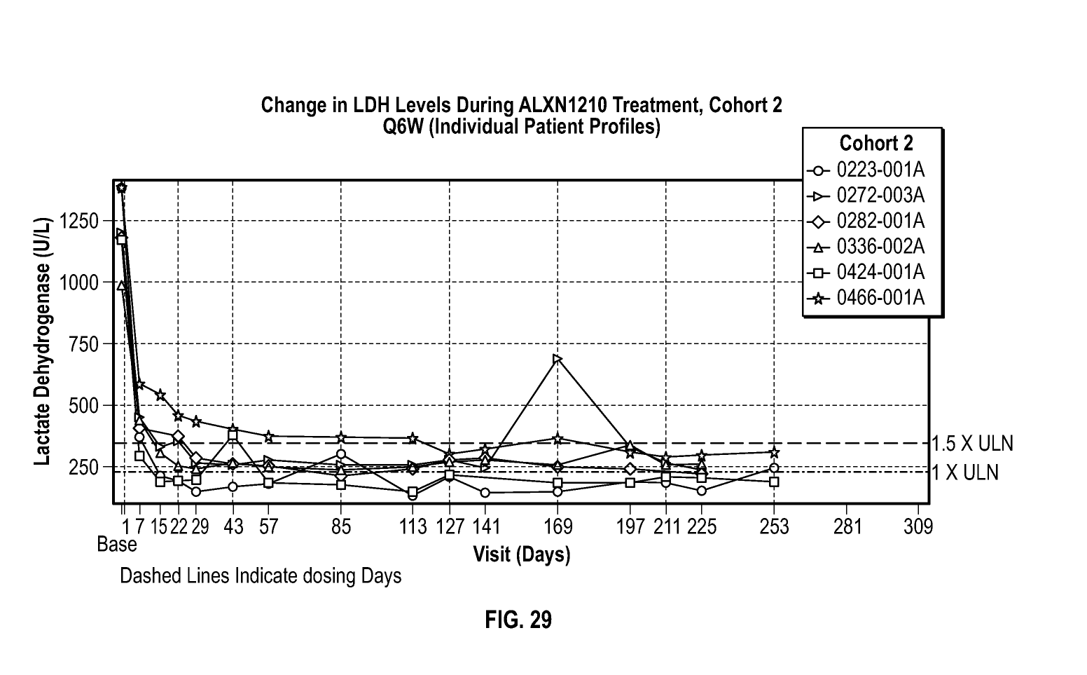

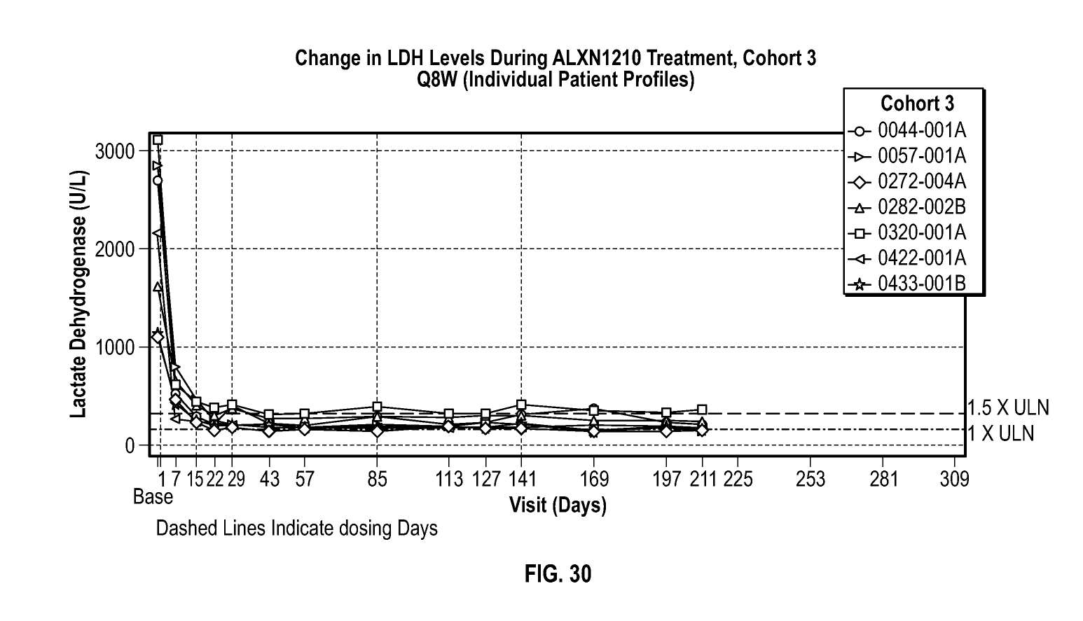

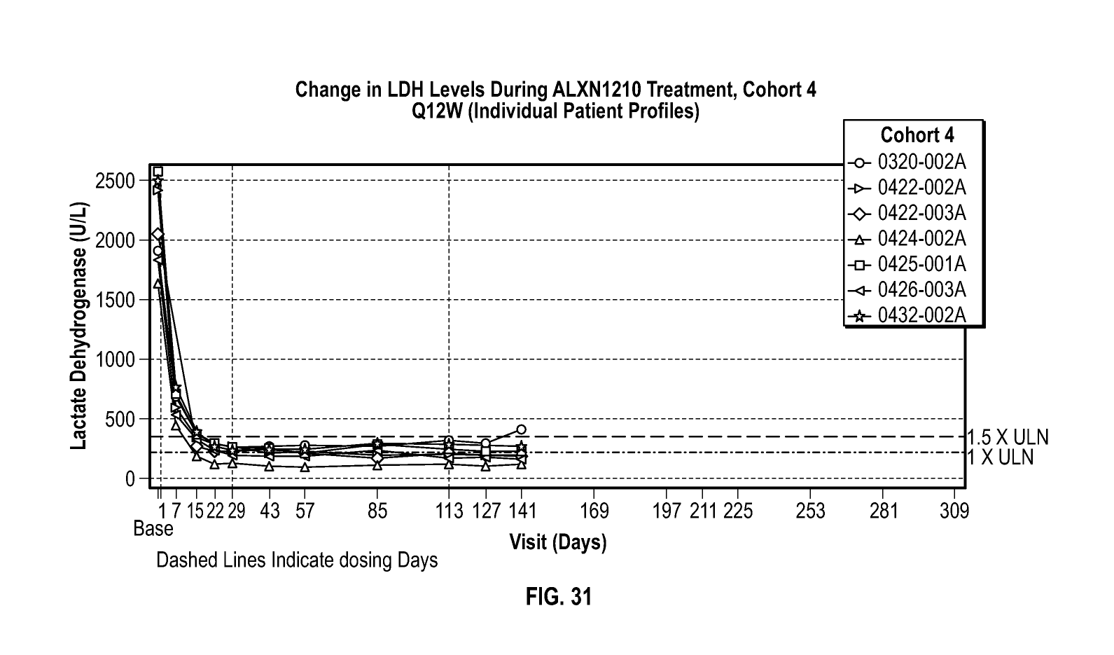

FIG. 27 depicts the change in LDH levels for all four Cohorts and FIGS. 28-31 depict the change in LDH levels for individual patient profiles within Cohort 1 (FIG. 28), Cohort 2 (FIG. 29), Cohort 3 (FIG. 30), and Cohort 4 (FIG. 31) during treatment with ALXN1210. As show in FIGS. 27-31, ALXN1210 treatment resulted in rapid and sustained reductions of LDH levels in patients with PNH previously naïve to complement inhibitor therapy.

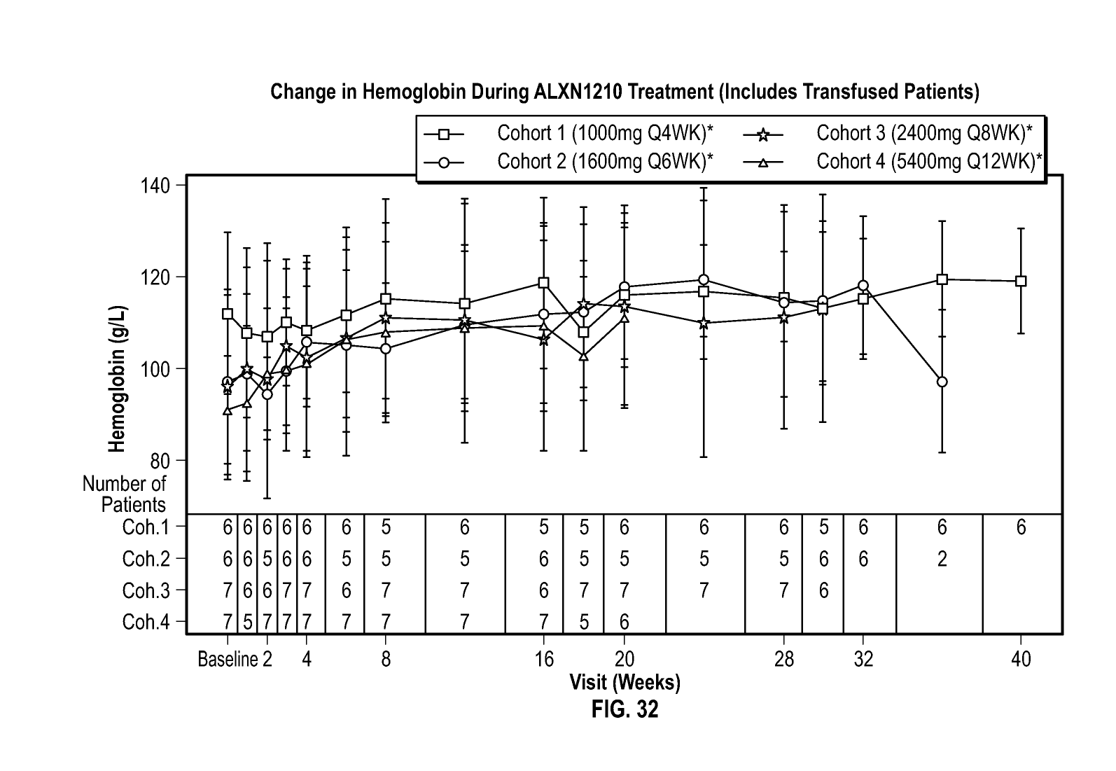

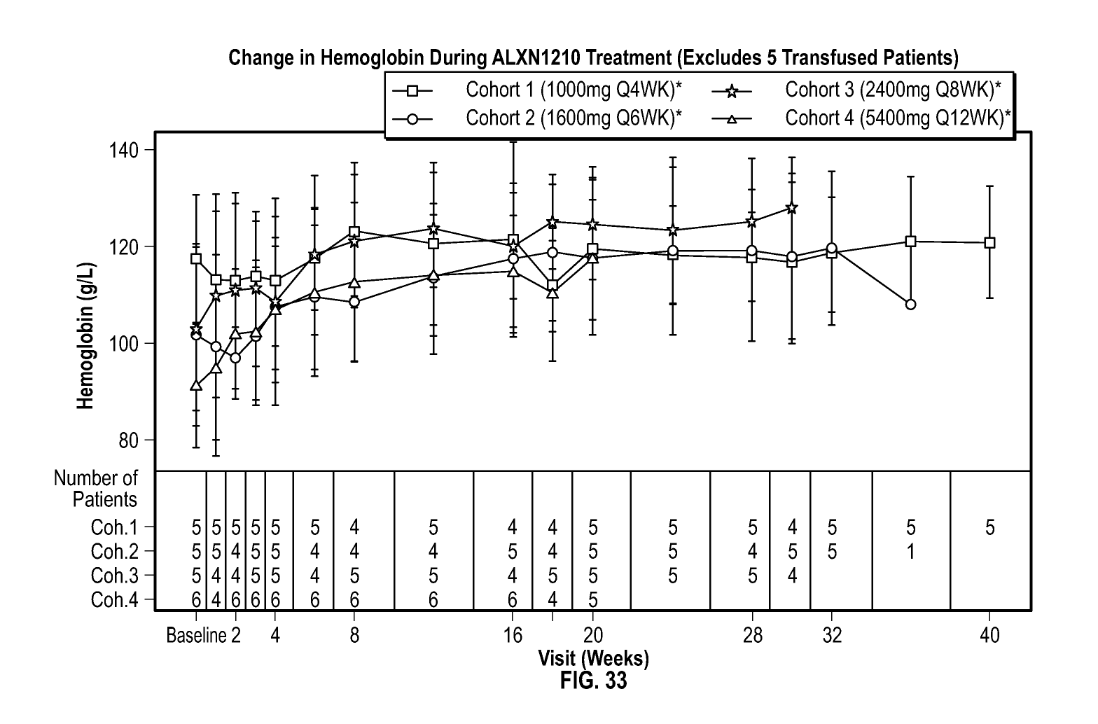

FIGS. 32 and 33 show the change in hemoglobin during treatment with ALXN1210, including transfused patients (FIG. 32) and excluding transfused patients (FIG. 33).

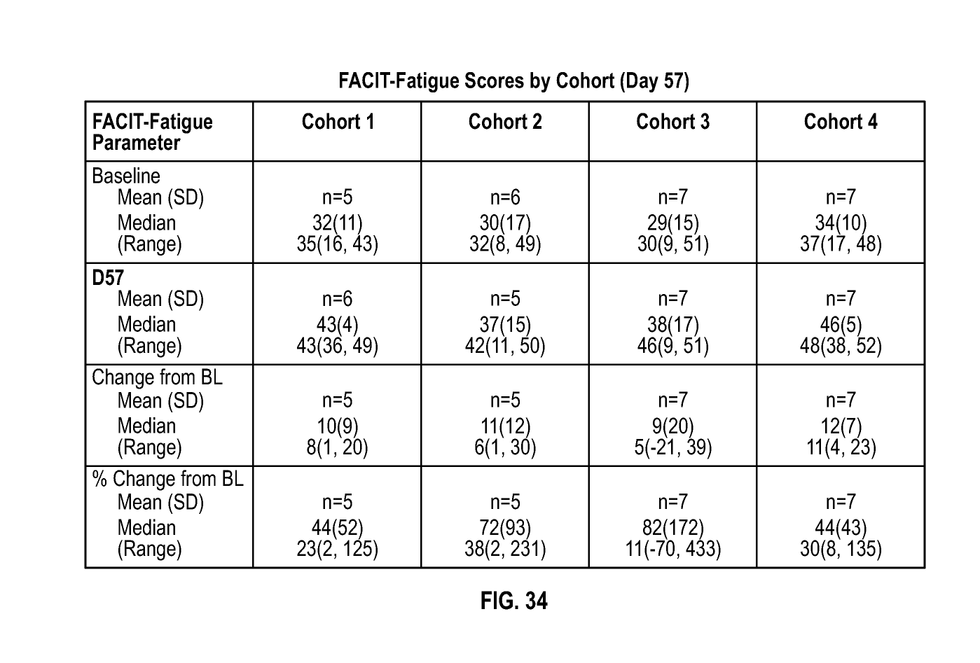

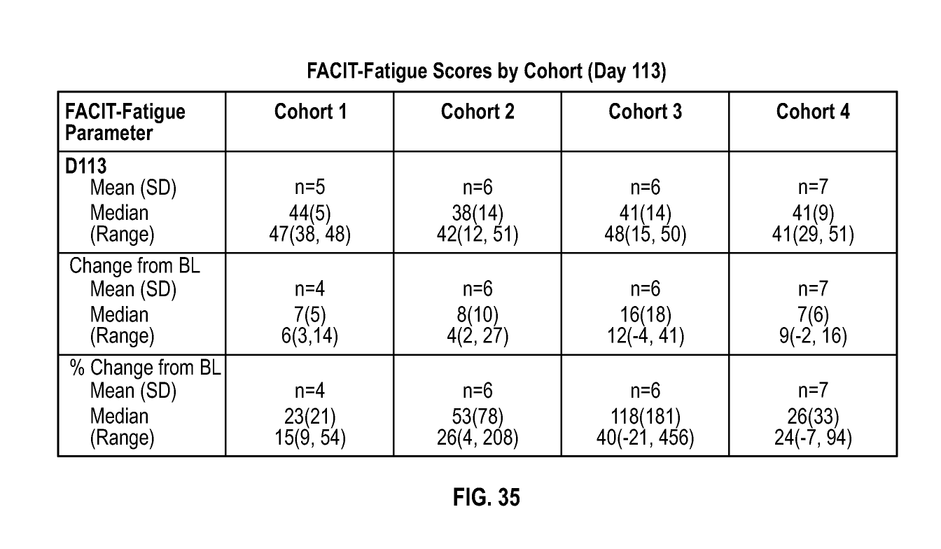

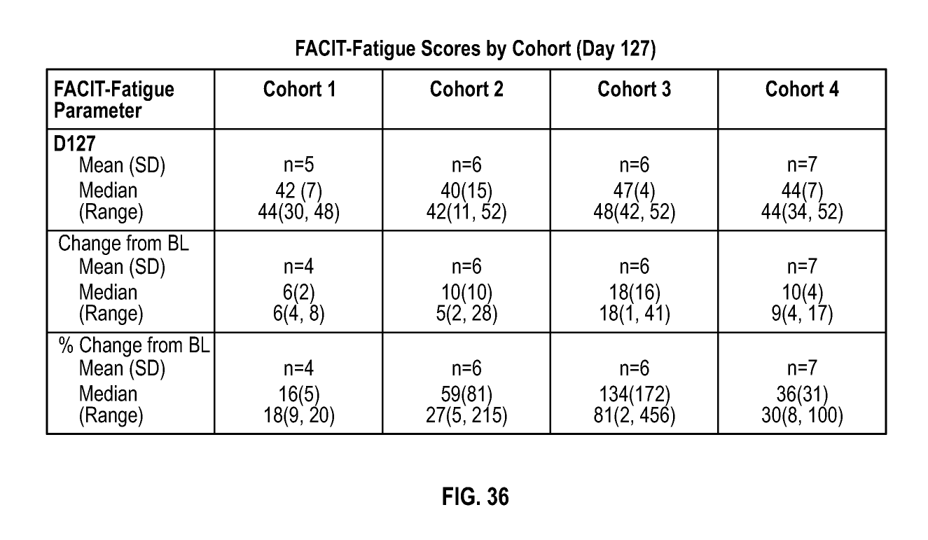

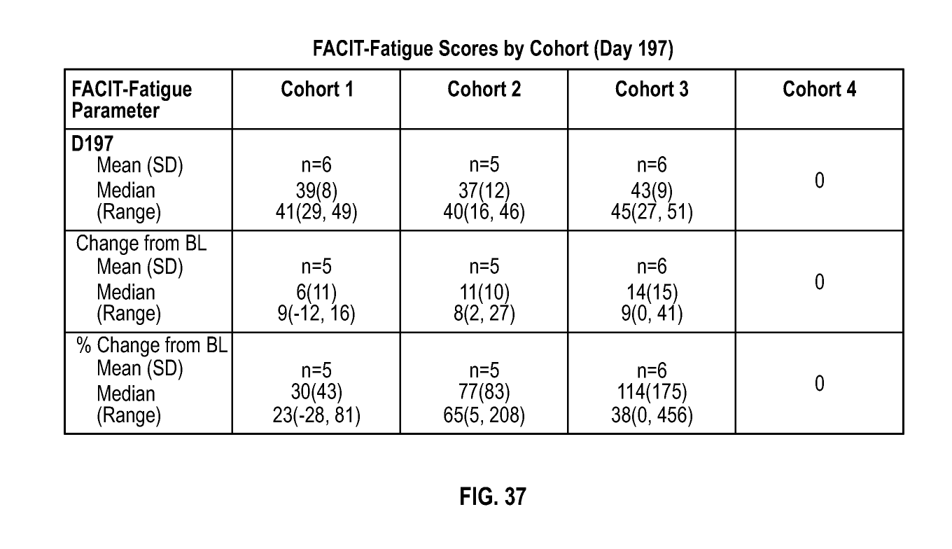

FIGS. 34-37 summarize the FACIT-Fatigue scores by cohort at Day 57 (FIG. 34), Day 113 (FIG. 35), Day 127 (FIG. 36) and Day 197 (FIG. 37). The mean increase in FACIT-Fatigue score was clinically significant (increase of at least three points) in all cohorts at all available post baseline time points.

SEQUENCE SUMMARY

SEQ ID NO: 1
amino acid sequence of heavy chain CDR1 of eculizumab (as defined under combined
Kabat-Chothia definition)
GYIFSNYWIQ
SEQ ID NO: 2
amino acid sequence of heavy chain CDR2 of eculizumab (as defined under Kabat
definition)
EILPGSGSTEYTENFKD
SEQ ID NO: 3
amino acid sequence of the heavy chain CDR3 of eculizumab (as defined under
combined Kabat definition).
YFFGSSPNWYFDV
SEQ ID NO: 4
amino acid sequence of the light chain CDR1 of eculizumab (as defined under Kabat
definition)
GASENIYGALN
SEQ ID NO: 5
amino acid sequence of light chain CDR2 of eculizumab
(as defined under Kabat definition)
GATNLAD
SEQ ID NO: 6
amino acid sequence of light chain CDR3 of eculizumab (as defined under Kabat
definition)
QNVLNTPLT
SEQ ID NO: 7
amino acid sequence of heavy chain variable region of eculizumab
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCARY
FFGSSPNWYFDVWGQGTLVTVSS
SEQ ID NO: 8
amino acid sequence of light chain variable region of eculizumab, BNJ441
antibody, and BNJ421 antibody
DIQMTQSPSSLSASVGDRVTITCGASENIYGALNWYQQKPGKAPKLLIYGA
TNLADGVPSRFSGSGSGTDFTLTISSLQPEDFATYYCQNVLNTPLTFGQGTK
VEIK
SEQ ID NO: 9
amino acid sequence of heavy chain constant region of eculizumab and BNJ421
antibody
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSQEDPEVQF
NWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
SEQ ID NO: 10
amino acid sequence of entire heavy chain of eculizumab
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 11
amino acid sequence of entire light chain of eculizumab, BNJ441 antibody, and
BNJ421 antibody
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPV
TKSFNRGEC
SEQ ID NO: 12
amino acid sequence of heavy chain variable region of BNJ441 antibody and BNJ421
antibody
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
amino acid sequence of heavy chain constant region of BNJ441 antibody
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDPE
VQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK
SEQ ID NO: 14
amino acid sequence of entire heavy chain of BNJ441 antibody
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGTQTYT
CNVDHKPSNTKVDKTVERKCCVECPPCPAPPVAGPSVFLFPPKPKDTLMISR
TPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSV [symbol] HEALH [symbol] HYTQKSLSLSLGK
SEQ ID NO: 15
amino acid sequence of IgG2 heavy chain constant region variant comprising YTE
substitutions
ASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKC
CVECPPCPAPPVAGPSVFLFPPKPKDTLYITREPEVTCVVVDVSHEDPEVQF
NWYVDGMEVHNAKTKPREEQFNSTFRVVSVLTVVHQDWLNGKEYKCKV
SNKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVF
SCSVMHEALHNHYTQKSLSLSPGK
SEQ ID NO: 16
amino acid sequence of entire heavy chain of eculizumab variant comprising heavy
chain constant region depicted in SEQ ID NO: 15 (above)
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK
SEQ ID NO: 17
amino acid sequence of light chain CDR1 of eculizumab (as defined under Kabat definition)
with glycine to histidine substitution at position 8 relative to SEQ ID NO: 4
GASENIYHALN
SEQ ID NO: 18
depicts amino acid sequence of heavy chain CDR2 of eculizumab in which serine at position
relative to SEQ ID NO: 2 is substituted with histidine
EILPGSGHTEYTENFKD
SEQ ID NO: 19
amino acid sequence of heavy chain CDR1 of eculizumab in which tyrosine at
position 2 (relative to SEQ ID NO: 1) is substituted with histidine
GHIFSNYWIQ
SEQ ID NO: 20
amino acid sequence of entire heavy chain of BNJ421 antibody
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMIS
RTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTV
DKSRWQEGNVFSCSV [symbol] HEALH [symbol] HYTQKSLSLSLGK
SEQ ID NO: 21
amino acid sequence of heavy chain CDR1 of the 7086 antibody
SYAIS
SEQ ID NO: 22
amino acid sequence of heavy chain CDR2 of the 7086 antibody
GIGPFFGTANYAQKFQG
SEQ ID NO: 23
amino acid sequence of heavy chain CDR3 of the 7086 antibody
DTPYFDY
SEQ ID NO: 24
amino acid sequence of light chain CDR1 of the 7086 antibody
SGDSIPNYYVY
SEQ ID NO: 25
amino acid sequence of light chain CDR2 of the 7086 antibody
DDSNRPS
SEQ ID NO: 26
amino acid sequence of light chain CDR3 of the 7086 antibody
QSFDSSLNAEV
SEQ ID NO: 27
amino acid sequence of the heavy chain variable region of the 7086 antibody
QVQLVQSGAEVKKPGSSVKVSCKASGGTFSSYAISVWRQAPGQGLEWMGGIGPF
FGTANYAQKFQGRVTITADESTSTAYMELSSLRSEDTAVYYCARDTPYFD
YWGQGTLVTVSS
SEQ ID NO: 28
amino acid sequence of the light chain variable region of the 7086 antibody
DIELTQPPSVSVAPGQTARISCSGDSIPNYYVYWYQQKPGQAPVLVIYDDSNRPSGI
PERFSGSNSGNTATLTISGTQAEDEADYYCQSFDSSLNAEVFGGGTKLTVL
SEQ ID NO: 29
amino acid sequence of heavy chain CDR1 of the 8110 antibody
NYIS
SEQ ID NO: 30
amino acid sequence of heavy chain CDR2 of the 8110 antibody
IIDPDDSYTEYSPSFQG
SEQ ID NO: 31
amino acid sequence of heavy chain CDR3 of the 8110 antibody
YEYGGFDI
SEQ ID NO: 32
amino acid sequence of light chain CDR1 of the 8110 antibody
SGDNIGNSYVH
SEQ ID NO: 33
amino acid sequence of light chain CDR2 of the 8110 antibody
KDNDRPS
SEQ ID NO: 34
amino acid sequence of light chain CDR3 of the 8110 antibody
GTYDIESYV
SEQ ID NO: 35
amino acid sequence of the heavy chain variable region of the 8110 antibody
EVQLVQSGAEVKKPGESLKISCKGSGYSFTNYISWVRQMPGKGLEWMGIIDPDDSY
EYSPSFQGQVTISADKSISTAYLQWSSLKASDTAMYYCARYEYGGFDI
WGQGTLVTVSS
SEQ ID NO: 36
amino acid sequence of the light chain variable region of the 8110 antibody
SYELTQPPSVSVAPGQTARISCSGDNIGNSYVHWYQQKPGQAPVLVIYKDNDRPSG [symbol]
ERFSGSNSGNT ATLTISGTQAEDEADYYCGTYDIESYVFGGGTKLTV L
SEQ ID NO: 37
amino acid sequence of heavy chain CDR1 of the 305LO5 antibody
SSYYVA
SEQ ID NO: 38
amino acid sequence of heavy chain CDR2 of the 305LO5 antibody
AIYTGSGATYKASWAKG
SEQ ID NO: 39
amino acid sequence of heavy chain CDR3 of the 305LO5 antibody
DGGYDYPTHAMHY
SEQ ID NO: 40
amino acid sequence of light chain CDR1 of the 305LO5 antibody
QASQNIGSSLA
SEQ ID NO: 41
amino acid sequence of light chain CDR2 of the 305LO5 antibody
GASKTHS
SEQ ID NO: 42
amino acid sequence of light chain CDR3 of the 305LO5 antibody
QSTKVGSSYGNH
SEQ ID NO: 43
amino acid sequence of the heavy chain variable region of the 305LO5 antibody
QVQLVESGGGLVQPGGSLRLSCAASGFTSHSSYYVAWVRQAPGKGLEWVGAIY
TGSGATYKASWAKGRFTISKDTSKNQVVLTMTNMDPVDTATYYCASDGGYDYP
THAMHYWGQGTLVTVSS
SEQ ID NO: 44
amino acid sequence of the light chain variable region of the 305LO5 antibody
DVVMTQSPSSLSASVGDRVTITCQASQNIGSSLAWYQQKPGQAPRLLIYGASK
THSGVPSRFSGSGSGTDFTLTISSLQPEDVATYYCQSTKVGSSYGNHFGGGTK
VEIK [symbol]
indicates data missing or illegible when filed

What is claimed is:

1. A method for the treatment of patients with Paroxysmal Nocturnal Hemoglobinuria (PNH) to reduce hemolysis, the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, wherein the method comprises an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

2. A method for the treatment of patients with Paroxysmal Nocturnal Hemoglobinuria (PNH) to reduce hemolysis, the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein the method comprises an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

3. A method for the treatment of patients with atypical hemolytic uremic syndrome (aHUS), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, wherein the method comprises an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

4. A method for the treatment of patients with atypical hemolytic uremic syndrome (aHUS), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein the method comprises an administration cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

5. A method of treating a human patient with Paroxysmal Nocturnal Hemoglobinuria (PNH), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, wherein the method comprises an administration cycle comprising an induction phase followed by a maintenance phase, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

6. A method of treating a human patient with Paroxysmal Nocturnal Hemoglobinuria (PNH), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

7. A method of treating a human patient with atypical hemolytic uremic syndrome (aHUS), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, and CDR1, CDR2, and CDR3 light chain sequences as set forth in SEQ ID NOs:4, 5, and 6, respectively, wherein the method comprises an administration cycle comprising an induction phase followed by a maintenance phase, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

8. A method of treating a human patient with atypical hemolytic uremic syndrome (aHUS), the method comprising administering to the patient an effective amount of an anti-C5 antibody, or antigen binding fragment thereof, comprising CDR1, CDR2, and CDR3 heavy chain sequences as set forth in SEQ ID NOs:19, 18, and 3, respectively, CDR1, CDR2, and CDR3 light chain sequences as set forth in SEQ ID NOs:4, 5, and 6, respectively, and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or three times at a dose of 5400 mg during the maintenance phase.

9. The method of any one of claims 1-4, wherein the anti-C5 antibody, or antigen binding fragment thereof, is also administered at a dose of 400 mg on Day 8 of the induction phase.

10. The method of claim 9, wherein the anti-C5 antibody, or antigen binding fragment thereof, is administered at a dose of:
(c) 400 mg on Day 1, 400 mg on Day 8, and 600 mg on Day 15 of the administration cycle during the induction phase; and
(d) 900 mg on Days 29, 57, 85, 113, and 141 of the administration cycle during the maintenance phase.

11. The method of any one of claims 1-4, wherein the anti-C5 antibody, or antigen binding fragment thereof, is administered at a dose of:
(a) 600 mg on Day 1 of the administration cycle and 600 mg on Day 15 of the administration cycle during the induction phase; and
(b) 900 mg on Days 29, 57, 85, 113, and 141 of the administration cycle during the maintenance phase.

12. The method of any one of claims 1-4, wherein the anti-C5 antibody, or antigen binding fragment thereof, is administered at a dose of:
(a) 600 mg on Day 1 of the administration cycle and 900 mg on Day 15 of the administration cycle during the induction phase; and
(b) 1800 mg on Days 29, 57, 85, 113, and 141 of the administration cycle during the maintenance phase.

13. The method of any one of claims 5-8, wherein the anti-C5 antibody, or antigen binding fragment thereof, is administered at a dose of:
(a) 1400 mg on Day 1 and 1000 mg on Day 15 of the administration cycle during the induction phase; and
(b) 1000 mg on Days 29, 57, 85, 113, 141, 169, 197, and 225 of the administration cycle during the maintenance phase.

14. The method of any one of claims 5-8, wherein the anti-C5 antibody, or antigen binding fragment thereof, is administered at a dose of:
(a) 2000 mg on Day 1 and 1600 mg on Day 22 of the administration cycle during the induction phase; and
(b) 1600 mg on Days 43, 85, 127, 169, and 211 of the administration cycle during the maintenance phase.

15. The method of any one of claims 5-8, wherein the anti-C5 antibody, or antigen binding fragment thereof, is administered at a dose of:
(a) 1600 mg on Day 1 and 1600 mg on Day 15 of the administration cycle during the induction phase; and
(b) 2400 mg on Days 29, 85, 141, and 197 of the administration cycle during the maintenance phase.

16. The method of any one of claims 5-8, wherein the anti-C5 antibody, or antigen binding fragment thereof, is administered at a dose of:
(a) 3000 mg on Day 1 of the administration cycle during the induction phase; and 5400 mg on Days 29, 113, and 197 of the administration cycle during the maintenance phase.

17. The method of any one of the preceding claims, wherein the anti-C5 antibody, or antigen-binding fragment thereof, comprises a heavy chain variable region depicted in SEQ ID NO:12 and a light chain variable region depicted in SEQ ID NO:8.

18. The method of any one of the preceding claims, wherein the anti-C5 antibody, or antigen-binding fragment thereof, further comprises a heavy chain constant region depicted in SEQ ID NO:13.

19. The method of any one of the preceding claims, wherein the antibody, or antigen-binding fragment thereof, comprises a heavy chain polypeptide comprising the amino acid sequence depicted in SEQ ID NO:14 and a light chain polypeptide comprising the amino acid sequence depicted in SEQ ID NO:11.

20. The method of any one of the preceding claims, wherein the anti-C5 antibody, or antigen-binding fragment thereof, binds to human C5 at pH 7.4 and 25° C. with an affinity dissociation constant (K_{D}) that is in the range 0.1 nM≤K_{D}≤1 nM.

21. The method of any one of the preceding claims, wherein the anti-C5 antibody, or antigen-binding fragment thereof, binds to human C5 at pH 6.0 and 25° C. with a K_{D}≥10 nM.
(b)

22. The method of any one of the preceding claims, wherein the treatment maintains a serum trough concentration of the anti-C5 antibody, or antigen binding fragment thereof, of 100 μg/ml or greater during the induction phase and/or the maintenance phase.

23. The method of any one of the preceding claims, wherein the treatment maintains a serum trough concentration of the anti-C5 antibody, or antigen binding fragment thereof, of 200 μg/ml or greater during the induction phase and/or the maintenance phase.

24. The method of any one of the preceding claims, wherein the anti-C5 antibody, or antigen binding fragment thereof, is administered on a monthly basis after the maintenance phase.

25. The method of any one of claims 1-4 and 9-12, wherein the anti-C5 antibody, or antigen binding fragment thereof, is administered at a dose of 900 mg or 1800 mg on a monthly basis after the maintenance phase for up to two years.

26. The method of any one of claims 1-4 and 9-12 wherein the administration cycle comprises a period of 21 weeks.

27. The method of any one of claims 5-8 and 13-16, wherein the anti-C5 antibody, or antigen binding fragment thereof, is administered at a dose of 1000 mg every four weeks, 1600 mg every six weeks, or 2400 mg every eight weeks after the maintenance phase.

28. The method of claim 13, wherein the anti-C5 antibody, or antigen binding fragment thereof, is administered at a dose of 1000 mg every four weeks after the maintenance phase for up to two years.

29. The method of claim 14, wherein the anti-C5 antibody, or antigen binding fragment thereof, is administered at a dose of 1600 mg every six weeks after the maintenance phase for up to two years.

30. The method of claim 15, wherein the anti-C5 antibody, or antigen binding fragment thereof, is administered at a dose of 2400 mg every eight weeks after the maintenance phase for up to two years.

31. The method of claim 16, wherein the anti-C5 antibody, or antigen binding fragment thereof, is administered at a dose of 5400 mg every twelve weeks after the maintenance phase for up to two years.

32. The method of any one of claims 5-8, 13-16, and 27-31, wherein the administration cycle comprises a period of 36 weeks.

33. The method of any one of the preceding claims, wherein the anti-C5 antibody, or antigen binding fragment thereof, is formulated for intravenous administration.

34. The method of any one of the preceding claims, wherein the patient has not previously been treated with a complement inhibitor.

35. The method of any one of the preceding claims, wherein the treatment results in terminal complement inhibition.

36. The method of any one of the preceding claims, wherein the treatment results in a reduction of hemolysis as assessed by lactate dehydrogenase (LDH) levels.

37. The method of claim 36, wherein the treatment results in at least about a 6 fold decrease in LDH levels four weeks after initiating treatment.

38. The method of claim 36, wherein the treatment results in about a 6, 7, 8, or 9 fold decrease in LDH levels four weeks after initiating treatment.

39. The method of claim 36, wherein the treatment results in at least about a 9 fold decrease in LDH levels six weeks after initiating treatment.

40. The method of claim 36, wherein the percent decrease in LDH levels from baseline is at least about 84% four weeks after initiating treatment.

41. The method of claim 36, wherein the percent decrease in LDH levels from baseline is about 84%, 85%, 86%, 87%, 88%, or 89%, four weeks after initiating treatment.

42. The method of claim 36, wherein the percent decrease in LDH levels from baseline is at least about 89% six weeks after initiating treatment.

43. The method of claim 36, wherein the treatment results in normal lactate dehydrogenase (LDH) levels or to within 10%, or within 20% above normal LDH levels.

44. The method of claim 43, wherein normal LDH levels are between 105-333 IU/L (international units per liter).

45. The method of claim 43 or 44, wherein the patient's LDH levels are ≥1.5 fold above the upper limit of normal (LDH≥1.5×ULN) prior to initiating treatment.

46. The method of any one of claims 1, 2, 5, and 6, wherein the treatment produces at least one therapeutic effect selected from the group consisting of a reduction or cessation in fatigue, abdominal pain, dyspnea, dysphagia, chest pain, and erectile dysfunction.

47. The method of any one of claims 3, 4, 7, and 8, wherein the treatment produces at least one therapeutic effect selected from the group consisting of a reduction or cessation in hypertension, proteinuria, uremia, lethargy/fatigue, irritability, thrombocytopenia, microangiopathic hemolytic anemia, and renal function impairment.

48. The method of any one of the preceding claims, wherein the treatment produces a shift toward normal levels of a hemolysis-related hematologic biomarker selected from the group consisting free hemoglobin, haptoglobin, reticulocyte count, PNH red blood cell (RBC) clone and D-dimer.

49. The method of any one of the preceding claims, wherein the treatment produces a reduction in the need for blood transfusions.

50. The method of any one of the preceding claims, wherein the treatment produces a reduction in major adverse vascular events (MAVEs).

51. The method of any one of the preceding claims, wherein the treatment produces a shift toward normal levels of a chronic disease associated biomarker selected from the group consisting estimated glomerular filtration rate (eGFR) and spot urine:albumin:creatinine and plasma brain natriuretic peptide (BNP).

52. The method of any one of the preceding claims, wherein the treatment produces a change from baseline in quality of life, assessed via the Functional Assessment of Chronic Illness Therapy (FACIT)-Fatigue Scale, version 4 and the European Organisation for Research and Treatment of Cancer, Quality of Life Questionnaire-Core 30 Scale,

53. A kit for treating Paroxysmal Nocturnal Hemoglobinuria (PNH) in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of claim 1, 2, 5, or 6

54. A kit for treating atypical hemolytic uremic syndrome (aHUS) in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of claim 3, 4, 7, or 8.

55. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration in a cycle comprising an induction phase followed by a maintenance phase, wherein:
(a) the induction phase comprises a period of three weeks, wherein the anti-C5 antibody, or antigen binding fragment thereof, is administered at a dose of 400 mg or 600 mg on Day 1 of the administration cycle and at a dose of 600 mg or 900 mg on Day 15 of the administration cycle; and
(b) the maintenance phase comprises a period of eighteen weeks, wherein the anti-C5 antibody, or antigen binding fragment thereof, is administered at a dose of 900 mg or 1800 mg on Days 29, 57, 85, 113, and 141 of the administration cycle.

56. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration in a cycle comprising an induction phase followed by a maintenance phase, wherein:
(a) the anti-C5 antibody, or antigen binding fragment thereof, is administered twice during the induction phase at a dose of 1000 mg, 1400 mg, 1600 mg, or 2000 mg or once during the induction phase at a dose of 3000 mg; and
(b) the anti-C5 antibody, or antigen binding fragment thereof, is administered eight times at a dose of 1000 mg, five times at a dose of 1600 mg, four times at a dose of 2400 mg, or 3 times at a dose of 5400 mg during the maintenance phase.

57. The antibody of claim 55 or 56, wherein the antibody is determined to be safe, tolerable, efficacious and sufficiently non-immunogenic after multiple IV doses for use in PNH or aHUS patients.


SEQUENCE LISTING (44 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK